(19) United States
(12) Patent Application Publication
(10) Pub. No.: US 2025/0090528 A1
(43) Pub. Date: Mar. 20, 2025

(54) USE OF COMPLEMENT FACTOR D INHIBITOR FOR TREATMENT OF LUPUS NEPHRITIS AND IMMUNOGLOBULIN A NEPHROPATHY

(71) Applicant: Alexion Pharmaceuticals, Inc., Boston, MA (US)

(72) Inventors: Nader Najafian, Chestnut Hill, MA (US); Katherine Gayle Garlo, Lynnfield, MA (US); Steven David Podos, New Haven, CT (US)

(73) Assignee: Alexion Pharmaceuticals, Inc., Boston, MA (US)

(21) Appl. No.: 18/700,270

(22) PCT Filed: Oct. 13, 2022
(86) PCT No.: PCT/US2022/046572

§ 371 Date: Apr. 11, 2024

Related U.S. Application Data
(60) Provisional application No. 63/256,898, filed on Oct. 18, 2021.

(51) Int. Cl. A61K 31/506; A61K 31/5377; A61K 31/573

(52) U.S. Cl. CPC A61K 31/506; A61K 31/5377; A61K 31/573

(57) ABSTRACT

Disclosed herein are methods for treating lupus nephritis (LN) and/or immunoglobulin A (IgA) nephropathy in a subject. The methods include administering to the subject a therapeutically effective amount of a small molecule complement factor D inhibitor.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition. While a properly functioning complement system provides a robust defense against infecting microbes, inappropriate regulation or activation of the complement pathways has been implicated in the pathogenesis of a variety of disorders, including lupus nephritis (LN) and immunoglobulin A nephritis (IgAN).

LN occurs in approximately 50% of patients with systemic lupus erythematosus (SLE) (Almaani et al., Adv Chronic Kidney Dis. 2019; 26(5): 393-403; Morales et al., Nephron. 2021; 145(1): 1-13), an autoimmune disorder caused by loss of tolerance to self-antigens, the production of autoantibodies, and deposition of complement-fixing immune complexes (ICs) in injured tissues (Bao et al., Kidney Dis (Basel). 2015; 1(2):91-99. The diagnosis of LN is determined by kidney biopsy according to the 2018 International Society of Nephrology/Renal Pathology Society (ISN/RPS) nomenclature and classification revised from the 2003 report (Bajema et al., Kidney Int. 2018; 93(4): 789-796; Markowitz et al., Kidney Int. 2017; 71(6): 491-495). In total, there are 6 classes of LN: Classes I to VI (Markowitz (2019), supra). The subset of patients with SLE that develop LN have the worst prognosis. LN leading to CKD is an independent major risk factor for overall mortality and morbidity attributed to cardiovascular diseases (Gasparotto et al., Rheumatology. 2020; 59(Suppl5): v39-v51). With current induction and maintenance therapies, the 5-year mortality is approximately 20%, and the risk of developing ESRD at 5, 10, and 15 years is 11%, 17%, and 22%, respectively (Tektonidou et al., Arthritis Rheumatol. 2016; 68(6): 1432-1441). Recurrence of LN after treatment (Renal Flare) occurs within 1 year in up to 25% of patients and is associated with increased risk of chronic kidney disease (CKD) progression (Almaani et al., Clin J Am Soc Nephrol. 2017; 12(5): 825-835). The American College of Rheumatology (ACR) and jointly the European League Against Rheumatism (EULAR) and European Renal Association-European Dialysis and Transplant Association (ERA-EDTA) recommend immunosuppression treatment for Class III, IV, III/V, and IV/V LN, also called “proliferative” LN (Bertsias et al., Ann Rheum Dis. 2012; 71(11): 1771-1782). The guidelines agree on induction treatment with glucocorticoids plus mycophenolate mofetil (MMF) or low-dose cyclophosphamide. For maintenance therapy, the guidelines agree on MMF or azathioprine, with or without low dose glucocorticoids (Bertsias (2012), supra). In patients with LN, the main goal of therapy is prevention of CKD progression, prevention of end-stage renal disease (ESRD), and improved survival. Lack of achievement of remission, in particular complete remission, is one of the major risk factors for the progression of renal disease. Hence, short-term CRR and partial renal response (PRR) are used to assess the efficacy of standard of care and novel therapies. After 6 to 12 months of treatment, only 10% to 40% of patients achieve a CRR with standard of care (Parikh et al., Am J Kidney Dis. 2020; 76(2): 265-281). Recent approvals in the US of belimumab and voclosporin represent progress in the treatment of LN. Results at two years showed a CRR of 30.0% for belimumab vs 20.0% for placebo Furie et al., N Engl K Med. 2020; 383(12): 1117-1128. For voclosporin, CRR was 40.8% compared to 22.5% for placebo at one year (Arriens et al., Ann Rheum Dis. 2020; 79 (Suppl 1): 172-173). However, a significant need exists for therapies that yield fast and durable responses, with high complete response rates, along with reduced need for steroids and/or immunosuppressants in patients with LN.

IgAN, also known as Berger's disease, is the most common global primary glomerulonephropathy that can progress to renal failure. IgAN is a lifelong disease leading to CKD and progresses to ESRD in 30% to 40% of patients over the course of 20 to 30 years (Lai et al., F1000Res. 2016; 5). Patients initially present with hematuria and hypertension, and proteinuria develops as the disease progresses. Diagnosis of IgAN is made by renal biopsy demonstrating immunoglobulin A (IgA) immunofluorescence in the glomeruli, usually codominant with C3 according to the Oxford Classification nomenclature (KDIGO, Kidney Int Suppl (2011). 2017; 7(1): 1-59; Rizk eta I., Front Immunol. 2019; 10: 504; Trimarchi et al, Kidney Int. 2017; 91(5): 1014-1021). Treatments for IgAN include RAS blocking agents, such as angiotensin-converting enzyme (ACE) inhibitors or angiotensin II receptor blockers (ARBs). These therapies are aimed at controlling blood pressure and preserving kidney function through decreasing intraglomerular pressure, which in turn reduces proteinuria. These treatments are insufficient in preserving renal function in patients with IgAN, as a high proportion still suffer from progressive CKD and ESRD (KDIGO clinical practice guideline on glomerular diseases (DRAFT), 2020). Patients with baseline hypertension (Pugh et al., Drugs. 2019; 79(4): 365-379) and proteinuria >1 g/day (Reich et al., J Am Soc Nephrol. 2007; 18(12): 3177-3183) are at increased risk for renal disease progression.

Accordingly, it is an object of the present disclosure to provide new therapies for treating LN and IgAN.

### SUMMARY OF THE DISCLOSURE

The present disclosure generally relates to the treatment of lupus nephritis (LN) and immunoglobulin A nephropathy (IgAN) with a small molecule Complement Factor D inhibitor. The disclosure is based, in part, on the recognition that Factor D inhibition, e.g., with compound 1 or a pharmaceutical salt thereof, resulting in direct blockage of complement AP activation and attenuation of complement activation via the amplification loop, has potential for therapeutic efficacy in patients with diseases such as LN and/or IgAN. The present disclosure contemplates that inhibition of FD in vivo, with effective amounts of orally administered compound 1 or pharmaceutical compositions thereof, is effective in a variety of clinical settings, e.g., induction treatment in actively proliferating disease and maintenance treatment in chronic disease.

Further embodiments of the disclosure relate to use of endpoints such as proteinuria and/or estimated glomerular filtration rates (eGFR) as markers of efficacy of compound 1 or pharmaceutical compositions thereof, in the effective treatment of LN and/or IgAN. Recognizing the anti-inflammatory effects of compound 1, the inventors conceived that treatment of patients suffering from LN/IgAN with compound 1 or pharmaceutical compositions thereof would lead to meaningful reduction in proteinuria, particularly, correlating with a reduction in hematuria in patients with IgAN and/or active Class III or IV nephritis on kidney biopsy in participants with LN. The additional efficacy parameter, eGFR of >30 mL/min/1.73 m², is indicative of renal function/health in both IgAN and LN cohorts.

The disclosure relates to one or more of the following non-limiting embodiments:

In a first aspect, the disclosure features a method of treatment, wherein the method comprises treating lupus nephritis (LN) and/or immunoglobulin A nephropathy (IgAN) in a subject, said treating comprising administering to the subject a therapeutically effective amount of Compound 1:

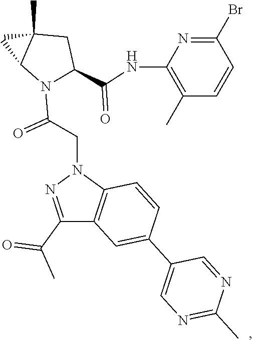 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof and reducing proteinuria in the subject from baseline.

In some embodiments, Compound 1 or the pharmaceutically acceptable salt thereof is administered at a dose of about 60 mg to about 300 mg twice daily (BID), e.g., about 120 mg BID or about 180 mg BID.

In some embodiments, said treating comprises reducing proteinuria (e.g., by greater than about 30%, by greater than about 50%) in the subject from baseline, e.g., following a 26-week or 50-week treatment period.

In some embodiments, said treating includes improving renal function in the subject. The improvement in renal function may be an increase in an eGFR from baseline in the subject, e.g., after a 26-week or 50-week treatment period, as calculated using Chronic Kidney Disease Epidemiology Collaboration (CKD-EPI). The improvement in renal function may also be a reduction in an increase in an eGFR from baseline in the subject, e.g., after a 26-week or 50-week treatment period relative to a control (e.g., untreated subjects or placebo-treated subjects), as calculated using CKD-EPI. The improvement in renal function may also include an improvement in creatinine clearance in the subject.

In some embodiments, the subject has LN (e.g., de novo LN or relapsing LN).

In some embodiments, in which the subject has LN, the subject has been diagnosed with active focal or diffuse proliferative LN class II or IV confirmed by a kidney biopsy obtained ≤6 months prior to treatment. The subject may also be exhibiting Class V disease.

In some embodiments, in which the subject has LN, the LN is clinically active LN which requires immunosuppression induction treatment.

In some embodiments, in which the subject has LN, the subject has proteinuria with urine protein:creatinine ratio (UPCR)≥1 g/g based on a 24-hour urine collection prior to treatment.

In some embodiments, in which the subject has LN, the time to first occurrence of UPCR≤0.5 g/g as measured by spot urine sample is reduced as compared to a control (e.g., untreated subjects or placebo-treated subjects).

In some embodiments, in which the subject has LN, the subject experiences partial renal response (PRR; as defined by the criteria set forth in Example 1), e.g., following a 26-week and/or 50-week treatment period.

In some embodiments, in which the subject has LN, the subject experiences complete renal response (CRR; as defined by the criteria set forth in Example 1), e.g., following a 26-week and/or 50-week treatment period.

In some embodiments, in which the subject has LN, said treating comprises reducing a time to first occurrence of UPCR≤0.5 g/g from baseline as measured by spot urine sample, as compared to a control (e.g., untreated subjects or placebo-treated subjects).

In some embodiments, in which the subject has LN, the subject has not started corticosteroid induction treatment prior to (e.g., within a period of up to 6 weeks prior to) treatment with Compound 1 or the pharmaceutically acceptable salt thereof.

In some embodiments, in which the subject has LN and has not started corticosteroid induction treatment within prior to (e.g., within a period of up to 6 weeks prior to) treatment with Compound 1 or the pharmaceutically acceptable salt thereof, the subject may be administered a cumulative dose of about 1 g of methylprednisolone IV in one or multiple divided doses within a period of up to 6 weeks prior to the start of treatment with Compound 1 or the pharmaceutically acceptable salt thereof.

In some embodiments, in which the subject has LN and has not started corticosteroid induction treatment prior to (e.g., within a period of up to 6 weeks prior to) treatment with Compound 1 or the pharmaceutically acceptable salt thereof, the subject has been administered an oral corticosteroid at a dose of about 0.5 mg/kg/day with a minimum dose of about 30 mg/day and a maximum dose of about 60 mg/day for a period of up to six weeks prior to the start of treatment with Compound 1 or the pharmaceutically acceptable salt thereof.

In some embodiments, in which the subject has LN and has not started corticosteroid induction treatment prior to (e.g., within a period of up to 6 weeks prior to) treatment with Compound 1 or the pharmaceutically acceptable salt thereof, said treating comprises co-administering an oral corticosteroid at a dose of about 30 mg/day to about 60 mg/day. The dose of the oral corticosteroid may be tapered to about 7.5 mg/day, e.g., after a 12-week, 26-week, or 50-week treatment period.

In some embodiments, in which the subject has LN and has not started corticosteroid induction treatment prior to (e.g., within a period of up to 6 weeks prior to) treatment with Compound 1 or the pharmaceutically acceptable salt thereof, the subject is administered a first dose of mycophenolate mofetil (MMF) about 1-1.5 g/day (or an equivalent dose of enteric-coated mycophenolic acid sodium (MPS)) in one or more doses after administration of the cumulative dose of about 1 g of methylprednisolone IV prior to (e.g., up to a period of 6 weeks prior to) treatment with Compound 1 or the pharmaceutically acceptable salt thereof, and is administered a second dose of MMF (or an equivalent dose of enteric-coated MPS) in one or more doses until 50 weeks after the start of treatment with Compound 1 or the pharmaceutically acceptable salt thereof. In some embodiments, the second dose is about 1-1.5 g/day. In some embodiments, the second dose is about 1-1.5 g/day until 1 week after the start of treatment with Compound 1 or the pharmaceutically acceptable salt thereof, after which the second dose is increased to about 2-3 g/day.

In some embodiments, in which the subject has LN, the subject has initiated corticosteroid induction treatment prior to (e.g., up to a period 6 weeks prior to) start of treatment with Compound 1 or the pharmaceutically acceptable salt thereof.

In some embodiments, in which the subject has LN and has initiated corticosteroid induction treatment prior to (e.g., up to a period of 6 weeks prior to) start of treatment with Compound 1 or the pharmaceutically acceptable salt thereof, the subject has received a cumulative dose of methylprednisolone IV of 1 g or an equivalent oral corticosteroid and is receiving a first dose of MMF of about 2 g/day (or an equivalent dose of enteric-coated MPS) in one or more doses prior to (e.g., up to a period of 6 weeks prior to) start of treatment with Compound 1 or the pharmaceutically acceptable salt thereof, the subject is not administered an additional dose of methylprednisolone IV or equivalent oral corticosteroid, and the subject is administered a second dose of MMF (or an equivalent dose of enteric-coated MPS) in one or more doses until 50 weeks after the start of treatment with Compound 1 or the pharmaceutically acceptable salt thereof. In some embodiments, the second dose of MMF is about 2 g/day. In some embodiments, the second dose of MMF is about 2 g/day and is adjusted to about 2-3 g/day before 4 weeks after the start of treatment with Compound 1 or the pharmaceutically acceptable salt thereof.

In some embodiments, in which the subject has LN and has initiated corticosteroid induction treatment prior to (e.g., up to a period of 6 weeks prior to) start of treatment with Compound 1 or the pharmaceutically acceptable salt thereof, the subject has received a cumulative dose of methylprednisolone IV of about 1 g or an equivalent oral corticosteroid and is receiving a first dose of MMF of <about 2 g/day (or an equivalent dose of enteric-coated MPS) in one or more doses prior to (e.g., up to a period of 6 weeks prior to) start of treatment with Compound 1 or the pharmaceutically acceptable salt thereof, and the subject is not administered an additional dose of methylprednisolone IV or equivalent oral corticosteroid, and the subject is administered a second dose of MMF (or an equivalent dose of enteric-coated MPS) in one or more doses until 50 weeks after the start of treatment with Compound 1 or the pharmaceutically acceptable salt thereof. In some embodiments, the second dose of MMF is about 1-1.5 g/day at the start of treatment with Compound 1 or the pharmaceutically acceptable salt thereof. In some embodiments, the second dose of MMF is about 1-1.5 g/day for a one-week period following the start of treatment with Compound 1 or the pharmaceutically acceptable salt thereof, after which the second dose of MMF is increased to about 2-3 g/day before 4 weeks after the start of treatment with Compound 1 or the pharmaceutically acceptable salt thereof.

In some embodiments, in which the subject has LN and has initiated corticosteroid induction treatment prior to (e.g., up to a period of 6 weeks prior to) treatment with Compound 1 or the pharmaceutically acceptable salt thereof, the subject has been receiving prednisone or a prednisone equivalent at a first dose prior to the start of treatment with Compound 1 or the pharmaceutically acceptable salt thereof, the first dose is maintained through the second day of treatment with Compound 1 or the pharmaceutically acceptable salt thereof, after which the subject is administered an oral corticosteroid (e.g., prednisone or a prednisone equivalent) at a dose of about 0.5 mg/kg/day with a minimum dose of about 30 mg/day and a maximum dose of about 60 mg/day. The dose of the oral corticosteroid may be tapered to about 7.5 mg/day, e.g., after a 12-week, 26-week, or 50-week treatment period.

In some embodiments, in which the subject has LN, said treating comprises reducing a risk of experiencing a renal flare in the subject, e.g., within a 50-week treatment period.

In some embodiments, in which the subject has LN, said treating comprises reducing a risk of experiencing an extrarenal systemic lupus erythematosus (SLE) flare in the subject, e.g., within a 50-week treatment period.

In some embodiments, in which the subject has LN, said treating comprises reducing a risk of treatment failure in the subject, e.g., within a 50-week treatment period.

In some embodiments, in which the subject has LN, said treating comprises reducing a risk of suboptimal response in the subject, e.g., within a 50-week treatment period.

In some embodiments, in which the subject has LN, said treating comprises reducing a level of serum albumin in the subject from baseline.

In some embodiments, in which the subject has LN, said treating comprises reducing the time to first CRR or PRR (e.g., as determined by spot UCPR) as compared to a control (e.g., untreated subjects or placebo-treated subjects).

In some embodiments, in which the subject has LN, said treating comprises reducing the time to first occurrence of UPCR>50% decrease from baseline (e.g., as determined by spot UCPR) as compared to a control (e.g., untreated subjects or placebo-treated subjects).

In some embodiments, in which the subject has LN, the subject exhibits an increase in FACIT-Fatigue total score from baseline, e.g., following a 26-week or 50-week treatment period.

In some embodiments, in which the subject has LN, a level of antibodies against double-stranded DNA (anti-dsDNA) and/or antibodies against C1q complement component (anti-C1q) in the subject is reduced from baseline, e.g., following a 26-week or 50-week treatment period.

In some embodiments, in which the subject has LN, the subject has not received treatment with cyclophosphamide ≤6 months prior to treatment with Compound 1 or the pharmaceutically acceptable salt thereof.

In some embodiments, in which the subject has LN, the subject has not received treatment with a calcineurin inhibitor ≤3 months prior to treatment with Compound 1 or the pharmaceutically acceptable salt thereof.

In some embodiments, in which the subject has LN, wherein the subject has not received treatment with a cumulative dose of intravenous (IV) methylprednisolone >about 3 g for active renal flare.

In some embodiments, in which the subject has LN, the subject has not received treatment with MMF >about 2 g/day (or an equivalent thereof, e.g., MPS) for ≥4 consecutive weeks for active renal flare prior to treatment of Compound 1 or the pharmaceutically acceptable salt thereof.

In some embodiments, in which the subject has LN, the subject has not received treatment with prednisone ≥about 0.5 mg/kg/day or an equivalent thereof for ≥4 consecutive weeks for active renal flare prior to treatment of Compound 1 or the pharmaceutically acceptable salt thereof.

In some embodiments, in which the subject has LN, the subject does not have uncontrolled hypertension (systolic blood pressure >160 mmHg or diastolic blood pressure >110 mmHg) on 2 or more measurements within a 6-week period prior to treatment with Compound 1 or the pharmaceutically acceptable salt thereof.

In some embodiments, in which the subject has LN, the subject does not have a history of or has clinically active SLE-related cerebritis, seizures, pericarditis, stroke, or stroke syndrome requiring treatment.

In some embodiments, in which the subject has LN, the subject does not have an inability to take or tolerate treatment with a corticosteroid, MMF, or MPS.

In some embodiments, in which the subject has LN, the subject is restricted from receiving treatment with a calcineurin inhibitor (e.g., voclosporin).

In some embodiments, the subject has IgAN.

In some embodiments, in which the subject has IgAN, the IgAN is primary IgAN confirmed by a kidney biopsy obtained prior to treatment (see Table 15 for the IgAN classification criteria). In some embodiments, the kidney is obtained more than 2 years prior to treatment, and the subject has hematuria as defined by 1+ blood based on urine dipstick or 10 red blood cells (RBCs)/high-power field (hp) microscopy on urine sediment.

In some embodiments, in which the subject has IgAN, the subject has been receiving treatment with a stable and optimal dose of a RAS inhibitor (e.g., maximum allowed or tolerated angiotensin-converting enzyme (ACE) inhibitor and/or angiotensin receptor blocker (ARB) dose) prior to treatment with Compound 1 or the pharmaceutically acceptable salt thereof and continues to receive treatment with the RAS inhibitor during treatment with Compound 1 or the pharmaceutically acceptable salt thereof. In some embodiments, the subject is restricted from receiving treatment with a second RAS inhibitor (e.g., an ACE inhibitor and/or an ARB) within a 50-week period following the start of treatment with Compound 1 or the pharmaceutically acceptable salt thereof.

In some embodiments, in which the subject has IgAN, the subject has been receiving treatment with a stable and optimal dose of a direct renin antagonist prior to treatment with Compound 1 or the pharmaceutically acceptable salt thereof and continues to receive treatment with the direct renin antagonist during treatment with Compound 1 or the pharmaceutically acceptable salt thereof. In some embodiments, the subject is restricted from receiving treatment with a second direct renin antagonist within a 50-week period following the start of treatment with Compound 1 or the pharmaceutically acceptable salt thereof.

In some embodiments, in which the subject has IgAN, the subject has controlled and stable blood pressure (i.e., <140/90 mmHg) over a 3-month period prior to treatment with Compound 1 or the pharmaceutically acceptable salt thereof.

In some embodiments, in which the subject has IgAN, the subject experiences partial remission, e.g., following a 26-week or 50-week treatment period.

In some embodiments, in which the subject has IgAN, said treating comprises attenuating or flattening the slope of eGFR computed from baseline to week 26 of a treatment period as compared to a control (e.g., untreated subjects or placebo-treated subjects). See, e.g., Barratt et al., Kidney Int Rep. 2019; 4: 1633-1637.

In some embodiments, in which the subject has IgAN, the subject was not diagnosed with rapid progressive glomerulonephritis as measured by an eGFR loss ≥30% over a period of 3 months prior to treatment with Compound 1 or the pharmaceutically acceptable salt thereof.

In some embodiments, in which the subject has IgAN, the subject does not have a secondary etiology of IgAN (e.g., systemic lupus erythematosus (SLE), cirrhosis, or celiac disease).

In some embodiments, in which the subject has IgAN, the subject does not have clinically active Henoch-Schonlein purpura (IgA vasculitis) requiring treatment.

In some embodiments, in which the subject has IgAN, the subject has not received treatment with prednisone >about 20 mg/day or an equivalent thereof for >14 consecutive days or any other immunosuppressant (e.g., azathioprine or cyclophosphamide) prior to (e.g., within 6 months prior to) treatment with Compound 1 or the pharmaceutically acceptable salt thereof. Prednisone equivalents and dose relationships between prednisone and its equivalents are known in the art.

In some embodiments, in which the subject has IgAN, the subject does not have blood pressure of ≥140/90 mmHg prior to treatment with Compound 1 or the pharmaceutically acceptable salt thereof confirmed on 2 measurements >30 minutes apart.

In some embodiments, in which the subject has IgAN, the subject does not have a body mass index ≥38 kg/m² prior to treatment with Compound 1 or the pharmaceutically acceptable salt thereof.

In some embodiments, in which the subject has IgAN, the subject is restricted from receiving treatment with hydroxychloroquine.

In some embodiments, in which the subject has IgAN, the subject is restricted from receiving treatment with an immunosuppressive agent (e.g., MMF).

In some embodiments, in which the subject has IgAN, the subject is restricted from receiving treatment with a systemic corticosteroid for >14 consecutive days.

In some embodiments, said treating comprises decreasing plasma Bb fragment of complement factor B (Bb) concentration and serum alternative pathway (AP) activity.

In some embodiments, said treating comprises reducing hematuria in the subject. Reducing hematuria in the subject may include a decrease in red blood cells (RBC) in urine from baseline, e.g., after a 26-week or 50-week treatment period. Reducing hematuria may also include achieving <10 RBCs/hpf.

In some embodiments, said treating includes improving a 36-Item Short Form Survey Instrument (SF-36) score in one or more of Physical Functioning, Physical, Bodily Pain, Vitality, General Health, Emotion, Mental Health, and Social Functioning from baseline.

In some embodiments, said treating comprises improving a EuroQol 5-dimension 5-level questionnaire (EQ-5D-5L) score in one or more of mobility, usual activities, self-care, pain/discomfort, and anxiety/depression.

In some embodiments, the subject has an eGFR <30 mL/min/1.73 m² as calculated using CKD-EPI.

In some embodiments, the subject has less than 50% tubular atrophy, glomerular sclerosis, or crescent formation in glomeruli on the most recent kidney biopsy obtained prior to treatment.

In some embodiments, the subject does not have a concomitant significant renal disease other than LN or IgAN on the most recent kidney biopsy obtained prior to treatment with Compound 1 or the pharmaceutically acceptable salt thereof.

In some embodiments, the subject does not have a history of solid organ or bone marrow transplant.

In some embodiments, the subject is restricted from receiving a solid organ or bone marrow transplant during a 50-week treatment period.

In some embodiments, the subject has not had a splenectomy and does not have functional asplenia.

In some embodiments, the subject does not have a history of seizure.

In some embodiments, the subject does not have a known or suspected complement deficiency unless the complement deficiency is attributable to LN or IgAN.

In some embodiments, the subject does not have a history of or have risk factors for Torsades de Pointes, a screening QT interval corrected using Fridericia's formula (QTcF)>450 msec when the subject is male or >470 msec when the subject is female or is receiving medication known to significantly increase the corrected QT interval (QTc).

In some embodiments, the subject does not have an alanine aminotransferase level of >2× upper limit of normal (ULN).

In some embodiments, the subject does not have a direct bilirubin level of >2×ULN.

In some embodiments, the subject has a hemoglobin A1C level of ≤7.0% prior to treatment with Compound 1 or the pharmaceutically acceptable salt thereof.

In some embodiments, the subject does not have a known or suspected history of drug or alcohol abuse or dependence within 1 year prior to treatment.

In some embodiments, the subject does not have a history of malignancy within 5 years prior to treatment, wherein the malignancy is not nonmelanoma skin cancer or carcinoma in situ of the cervix that has been treated with no evidence of recurrence.

In some embodiments, the subject is not exhibiting signs of a hepatitis B viral infection with negative surface antibodies.

In some embodiments, the subject is not exhibiting signs of a hepatitis C viral infection; or is exhibiting signs of a hepatitis C viral infection but has been successfully treated and has a documented sustained virologic response.

In some embodiments, the subject is not exhibiting signs of a human immunodeficiency virus infection.

In some embodiments, the subject does not have bone marrow insufficiency or thrombocytopenia.

In some embodiments, the subject did not have an active systemic bacterial, viral, or fungal infection within 14 days prior to treatment.

In some embodiments, the subject does not have a history of N meningitidis infection.

In some embodiments, the subject is not receiving treatment with a biologic medication that may affect immune system functioning (e.g., a corticosteroid or an immunosuppressant); or has stopped receiving treatment with the biologic medication, and 5 terminal half-lives of the biologic has elapsed prior to treatment with Compound 1 or the pharmaceutically acceptable salt thereof.

In some embodiments, the subject has not received treatment with belimumab or rituximab <6 months prior to treatment with Compound 1 or the pharmaceutically acceptable salt thereof.

In some embodiments, the subject has not received, or is not receiving treatment with a complement inhibitor other than Compound 1 or the pharmaceutically acceptable salt thereof.

In some embodiments, the subject has not received treatment with a medication selected from a strong cytochrome P450, family 3, subfamily A (CYP3A) inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment with Compound 1 or the pharmaceutically acceptable salt thereof.

In some embodiments, the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

In some embodiments, the subject is not pregnant or breastfeeding.

In some embodiments, the subject is restricted from consuming foods and beverages that inhibit CYP3A4 enzyme activity.

In some embodiments, the subject is restricted from receiving treatment with eculizumab.

In some embodiments, the subject is restricted from receiving treatment with ravulizumab.

In some embodiments, the subject has been receiving treatment with a sodium-glucose cotransporter-2 (SGLT-2) inhibitor on a stable dose (e.g., for 3 months) prior to treatment with Compound 1 or the pharmaceutically acceptable salt thereof, and the dose of the SGLT-2 inhibitor does not change during treatment (e.g., through a 50-week treatment period) with Compound 1 or the pharmaceutically acceptable salt thereof.

In some embodiments, the subject has not been receiving treatment with a SGLT-2 inhibitor prior to treatment with Compound 1 or the pharmaceutically acceptable salt thereof, and the subject is restricted from receiving treatment with a SGLT-2 inhibitor within a 50-week period following the start of treatment with Compound 1 or the pharmaceutically acceptable salt thereof.

In some embodiments, the subject is restricted from using a medication selected from a strong CYP3A inhibitor, a moderate CYP3A inhibitor, a strong inducer of CYP3A, a moderate inducer of CYP3A, and a sensitive substrate of CYP3A.

In some embodiments, the subject is restricted from using a medication selected from meperidine, pethidine, a typical (1^{st} generation) antipsychotic, clozapine, olanzapine, lithium, a tricyclic antidepressant, bupropion, aminophylline, and theophylline.

In some embodiments, the subject is restricted from using a medication known to significantly prolong QTc, provided that the medication is not hydroxychloroquine for use by a subject with LN.

In another aspect, the present disclosure features a use of Compound 1 or a pharmaceutically acceptable salt thereof in the manufacture of a medicament for use in a method of treatment, wherein the method of treatment is any one of the methods disclosed herein.

In another aspect, the present disclosure features a compound for use in a method of treatment, wherein the compound is Compound 1 or a pharmaceutically acceptable salt thereof, wherein the method of treatment is any one of the methods disclosed herein.

In another aspect, the present disclosure features a kit for treating LN or IgAN in a subject, which includes (a) a dose of Compound 1 or a pharmaceutically acceptable salt thereof; and (b) instructions for using Compound 1 or the pharmaceutically acceptable salt thereof according to any one of the methods disclosed herein.

### BRIEF DESCRIPTIONS OF THE DRAWING

FIG. 1 is a schematic depicting the design of a Phase 2 clinical trial (lupus nephritis cohort) described in Example 1 (LN: lupus nephritis; bid: twice daily).

FIG. 2 is a schematic depicting the design of a Phase 2 clinical trial (immunoglobulin A nephropathy cohort) described in Example 1 (IgAN: immunoglobulin A nephropathy; bid: twice daily).

FIG. 3 shows the 2019 European League Against Rheumatism (ELAR)/American College of Rheumatology (ACR) Classification for systemic lupus erythematosus (SLE) used for determining eligibility for the lupus nephritis cohort described in Example 1.

### DETAILED DESCRIPTION

### Definitions

As used herein, the word “a” or “plurality” before a noun represents one or more of the particular nouns. For example, the phrase “a mammalian cell” represents “one or more mammalian cells.” The singular form “a,” “an,” and “the” include plural reference unless the context clearly dictates otherwise.

The term “about”, particularly in reference to a given quantity or number, is meant to encompass deviations within plus or minus ten percent (±10%), (e.g., ±5%).

As used herein, the term “baseline” refers to a parameter (e.g., a level, a score, or an anatomical measure) detected or measured in a subject at the start of a treatment regimen (e.g., in accordance with any one of the methods disclosed herein).

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “pharmaceutical composition” refers to an active compound, formulated together with one or more pharmaceutically acceptable excipients. In some embodiments, a compound is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained release formulation; topical application, for example, as a cream, ointment, or a controlled release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials

As used herein, the term “subject” or “patient” is a human patient (e.g., a patient having LN or IgA nephropathy). As used herein, the terms “subject” and “patient” are interchangeable.

As used herein, the term “treating” includes therapeutic treatments. The term “therapeutic” treatment is art-recognized and includes administration to a human subject of one or more of the disclosed compounds or formulations after manifestation of the unwanted condition (i.e., it is intended to diminish, ameliorate, or stabilize the existing unwanted condition or side effects thereof). Preferably, it is intended that the severity of the subject's condition (e.g., LN or IgAN) is reduced or at least partially improved or modified and that some alleviation, mitigation, reversal or decrease in at least one clinical symptom (e.g., proteinuria) is achieved.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to, for example, alleviation of at least one symptom of the disease or disorder (e.g., LN or IgAN).

As used herein, the term “effective amount” or “therapeutically effective amount” refers to an amount of an agent that provides the desired biological, therapeutic, and/or prophylactic result. That result can be reduction, amelioration, palliation, lessening, delaying and/or alleviation of one or more of the signs, symptoms, or causes of a disease, or any other desired alteration of a biological system. In one example, an “effective amount” is the amount of Compound 1 or a pharmaceutically acceptable salt thereof useful, e.g., clinically proven, to alleviate at least one symptom of the disease (e.g., LN or IgAN). An effective amount can be administered in one or more administrations.

As used herein, the term “reducing a risk of experiencing a renal flare” refers to reducing the frequency of occurrences of a renal flare (e.g., as defined by the criteria provided in Example 1) in subjects treated according to a method disclosed herein. The reduction is in comparison to control subjects (e.g., untreated subjects or placebo-treated subjects) of the same age, sex and/or condition (e.g., comorbidities). In some embodiments, the frequency of occurrences of renal flare in subjects treated according to a method disclosed herein is reduced by at least 10% (e.g., at least 20%, at least 30%, at least 40%, at least 50%, at least 60%, at least 70%, at least 80%, at least 90%, or 99% or more) relative to control subjects (e.g., untreated subjects or placebo-treated subjects).

As used herein, the term “reducing a risk of experiencing an extrarenal systemic lupus erythematosus (SLE) flare” refers to reducing the frequency of occurrences of extrarenal SLE flare (e.g., as defined by the criteria provided in Example 1) in subjects treated according to a method disclosed herein. The reduction is in comparison to control subjects (e.g., untreated subjects or placebo-treated subjects) of the same age, sex and/or condition (e.g., comorbidities). In some embodiments, the frequency of occurrences of extrarenal SLE flare in subjects treated according to a method disclosed herein is reduced by at least 10% (e.g., at least 20%, at least 30%, at least 40%, at least 50%, at least 60%, at least 70%, at least 80%, at least 90%, or 99% or more) relative to control subjects (e.g., untreated subjects or placebo-treated subjects).

As used herein, the term “reducing a risk of suboptimal response refers to reducing the frequency of occurrences of suboptimal response (as defined in Example 1) in subjects treated according to a method disclosed herein. The reduction is in comparison to control subjects (e.g., untreated subjects or placebo-treated subjects) of the same age, sex and/or condition (e.g., comorbidities). In some embodiments, the frequency of occurrences of suboptimal response in subjects treated according to a method disclosed herein is reduced by at least 10% (e.g., at least 20%, at least 30%, at least 40%, at least 50%, at least 60%, at least 70%, at least 80%, at least 90%, or 99% or more) relative to control subjects (e.g., untreated subjects or placebo-treated subjects).

As used herein, the term “reducing a risk of treatment failure” refers to reducing the frequency of occurrences of treatment failure (as defined in Example 1) in subjects treated according to a method disclosed herein. The reduction is in comparison to control subjects (e.g., untreated subjects or placebo-treated subjects) of the same age, sex and/or condition (e.g., comorbidities). In some embodiments, the frequency of occurrences of treatment failure in subjects treated according to a method disclosed herein is reduced by at least 10% (e.g., at least 20%, at least 30%, at least 40%, at least 50%, at least 60%, at least 70%, at least 80%, at least 90%, or 99% or more) relative to control subjects (e.g., untreated subjects or placebo-treated subjects).

As used herein, the term “proteinuria,” refers to the presence of protein in an abnormal amount in a subject's sample, e.g., urine sample. A variety of methods may be used in measuring urine protein levels. In some embodiments, in subjects with LN, proteinuria may be measured by urinary protein to creatinine ratio (UPCR), wherein urine is obtained from the patient once in a 24-hour period. In addition, urine albumin to creatinine ratio (UACR) may also be used. An exemplary MMRM method for measuring log-transformed proteinuria is outlined in the Examples. In some embodiments, in subjects with IgAN, proteinuria may be measured via measurement of actual protein levels, wherein urine is collected twice within a 24-hour window, and proteinuria is estimated from rate of creatinine excretion. As used herein, the term “reducing proteinuria’ refers to reducing the 24-hour urine protein excretion in a subject suffering from proteinuria (e.g., by at least 20%, at least 30%, at least 40%, at least 50%, at least 60%, at least 70%, at least 80%, at least 90%, or more) compared to baseline 24-hour urine protein excretion in the subject prior to treatment with an agent, e.g., with Compound 1 or a pharmaceutically acceptable salt thereof.

### Lupus Nephritis

The present disclosure provides methods for treating LN. The pathophysiology of LN involves multiple overlapping pathways where complement serves as a mediator of an abnormal immune response (Bao (2015), supra; Pickering et al., Rheumatology (Oxford). 2000; 39(2): 133-141). Immune complexes can activate the complement CP by direct interaction with complement component 1q (C1q) in the C1 complex; further activation by the AP-mediated amplification loop contributes to the overall accumulation of complement activation products and the resulting inflammatory response and tissue injury. The terminal complement components (C5a and terminal complement complex [C5b-9]) trigger acute cellular inflammatory responses through activation of interleukin and cytokine signaling. Complement also serves to fix immunoglobulins and ICs in the kidney (Thurman et al., Adv Chronic Kidney Dis. 2020; 27(2): 86-94). In fact, complement and complement split products are a prominent histologic finding in kidney biopsies of patients with LN (Biesecker et al., J Exp Med. 1981; 154(6)L 1779-1794; Wilson et al., Kidney Int. 2019; 95(3): 655-665. Serum levels of these autoimmune and complement biomarkers are linked with disease activity (Birmingham et al., Nephrol., Dial, Transplant. 2017; 32(suppl_1): i71-i79; Dall'Era et al., Arthritis Care Res (Hoboken). 2011; 63(3): 351-357). Decreases in C3, complement component 4 (C4), and C1q are associated with de novo LN and LN flares. Likewise, levels of complement biomarkers correlate with disease activity in SLE (Kim et al., Arthritis Rheumatol. 2019; 71(3): 420-430; Song et al., Am J Med Sci. 2017; 353(3): 247-257). In fact, the pharmacodynamic (PD) marker of Compound 1, plasma complement factor Bb, is significantly elevated in patients with active LN when compared to patients with LN in clinical remission, active SLE without LN, and normal controls. Lastly, plasma Bb level significantly correlates with kidney disease activity indices and is a risk factor for adverse kidney outcomes (Song (2017), supra).

Restoring complement regulation may improve renal responses through acute anti-inflammatory effects and lasting effects on IC deposition-mediated injury in the kidney. The contribution of AP activity to LN pathophysiology has been demonstrated in mouse models (Elliot et al., Kidney Int. 2004; 65(1): 129-138; Grossman et al., Immunobiology. 2016; 221(6): 701-708; Watanabe et al., J Immunol. 2000; 164(2): 786-794) and further supported by soluble biomarker profiles and the composition of renal deposition in patients with LN (Lukawska et al., Clin Exp Med. 2018; 18(3): 297-318; Kim et al., Lupus. 2020; 29(8): 862-871; Birmingham (2017), Song (2017), supra). Restoring AP regulation may improve renal responses through acute anti-inflammatory effects and lasting effects on IC deposition-mediated injury in the kidney. Hence, FD inhibition is promising for both induction treatment for active proliferative LN and maintenance treatment of chronic LN (Thurman et al., J Immunol. 2006; 176(3):1305-1310; Lukawska (2018), supra).

### Immunoglobulin A Nephropathy

The present disclosure provides methods for treating IgAN. The pathophysiology of IgAN is related to the overproduction of under-glycosylated immunoglobulin A1 (IgA1), which accumulates in the kidney glomeruli. However, aberrant galactosylation alone is insufficient to induce renal injury; glycan-specific IgA and immunoglobulin G (IgG) autoantibodies that recognize the under-galactosylated IgA1 molecule likely also contribute. This process leads to the local inflammation and complement activation in the kidney (Oortwijn et al., Semin Nephrol. 2008; 28(1): 58-65). Both the AP and LP complement pathways may be activated, leading to generation of anaphylatoxins of C3a and C5a, and the MAC C5b-9, with subsequent promotion of inflammatory mediators (Maillard et al., J Am Soc Nephrol. 2015; 26(7): 1503-1512; Lukawska (2018), Thurman (2006), supra). C4 and C3 complexes and activated C3 products are elevated in up to 30% of patients with IgAN. Activated C3 products are associated with elevated levels of proteinuria and hematuria compared to patients with IgAN who have normal levels of these products, and correlate with deterioration of renal function (Zwirner et al., Kidney Int. 1997; 51(4): 1257-1264). Complement activity on kidney biopsy and circulating complement proteins are associated with disease activity and progression of CKD. Evidence for AP involvement has been established in the codeposition with IgA of properdin and of regulators of AP C3 convertase stability including complement factor H (FH) and factor H-related protein-5 (FHR5) (Medjeral-Thomas et al., Clin J Am Soc Nephrol. 2014; 9(1): 46-53; Rizk (2019, supra). Further support has arisen from the association of IgAN pathogenesis with circulating levels of the FHR proteins, and with the identification of both protective and pathogenic variants in the CFH locus which affect expression or activity of FH and FH-related proteins (Tortajada et al., Mol Immunol. 2019; 114: 123-132). Together these findings suggest a role of complement in the pathophysiology and the prognostic value of complement biomarkers in IgAN (Rizk (2019), supra). Animal studies demonstrate that aberrantly glycosylated IgA complexes with C3b and FB are codeposited in glomeruli (Hashimoto et al., Am J Pathol. 2012; 181(4): 1338-1347). This is supported in humans through data demonstrating elevated messenger ribonucleic acid (mRNA) transcript expression of FD and properdin in glomeruli relative to other kidney fractions (Song et al., Nephron. 1998; 78(1): 15-22).

Factor D inhibition is a potential target for treatment of patients with IgAN at high risk of progression to kidney disease (i.e., significant proteinuria despite optimal RAS blockade) (Reich et al., J am Soc Nephrol. 2007; 18(12): 3177-3183; Rizk (2019), supra). Factor D inhibition blocks complement AP activation directly and tempers complement activation via other pathways through inhibition of the amplification loop, leading to prevention of the downstream molecular and cellular consequences, and subsequently has potential for therapeutic efficacy in patients with IgAN.

### Method of Treatment

The present disclosure provides methods for treating a subject suffering from LN and/or IgAN by administering to the subject a therapeutically effective amount of Compound 1:

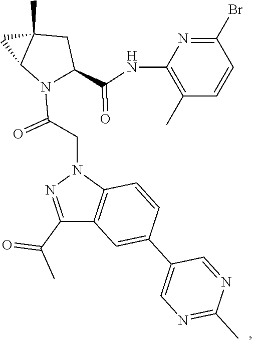 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, Compound 1 or the pharmaceutically acceptable salt thereof is administered at a dose of about 60 mg to about 300 mg twice daily (BID), e.g., about 120 mg BID or about 180 mg BID. In some embodiments, Compound 1 or the pharmaceutically acceptable salt thereof is administered at a dose of about 180 mg BID.

In some embodiments, the course of treatment with Compound 1 or a pharmaceutically acceptable salt thereof lasts for 26 weeks. In some embodiments, the course of treatment with Compound 1 or a pharmaceutically acceptable salt thereof lasts for 50 weeks. In some embodiments, the course of treatment lasts for 26-52, 26-78, 26-104, 26-130, 26-154 weeks, or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 102, 130, 154 or 182 weeks. In some embodiments, the course of treatment lasts for greater than 1, 2, 3, 4, 5, 10, 15, 20, 25, 30, 35, 40, 45, 50, 55, 60, 65, 70, 75, 80, or more years. In some embodiments, the course of treatment lasts for the remainder of the subject's life.

In some embodiments, the first sign of improvement occurs by 12 weeks of treatment with Compound 1 or a pharmaceutically acceptable salt thereof. In some embodiments, the first sign of improvement occurs by 26 weeks of treatment with Compound 1 or a pharmaceutically acceptable salt thereof. In some embodiments, the first sign of improvement occurs by 50 weeks of treatment with Compound 1 or a pharmaceutically acceptable salt thereof. In some embodiments, the first sign of improvement occurs between weeks 1-26, 26-52, 52-78, 78-102, 102-130, 130-156, 156-182, or 182-208 of treatment with Compound 1 or a pharmaceutically acceptable salt thereof. In some embodiments, the first sign of improvement occurs after 1, 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 100, 104, 108, or 154 weeks of treatment.

In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 100, 102, 104, 108, or 154 weeks of treatment, as measured by a urine protein to creatinine ratio derived from a 24-hour urine collection from the subject and/or by absolute protein derived from a mean of two 24-hour urine collections. In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline following a 154-week treatment period. In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline, following a 102-week treatment period. In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline, following a 50-week treatment period. In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline, following a 26-week treatment period. In some embodiments, said treating includes reducing proteinuria (e.g., by greater than about 30%, by greater than about 40%, by greater than about 50%, by greater than about 60%, by greater than about 70%, by greater than about 80%, by greater than about 90%, or more) in the subject from baseline, following a 9-week treatment period. In some embodiments, said treating includes reducing proteinuria by greater than 30% (e.g., greater than 50%) after a 50-week treatment period. In some embodiments, said treating includes reducing proteinuria by greater than 30% (e.g., greater than 50%) after a 26-week treatment period.

In some embodiments, said treating includes improving renal function in the subject, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. The improvement in renal function may be an increase in an eGFR from baseline (e.g., by about 10%, by about 20%, by about 30%, by about 40%, but about 50%, by about 60%, by about 70%, by about 80%, by about 90%, or more) in the subject as calculated using Chronic Kidney Disease Epidemiology Collaboration (CKD-EPI) The improvement in renal function may also be a reduction in an increase in an eGFR from baseline (e.g., by about 10%, by about 20%, by about 30%, by about 40%, but about 50%, by about 60%, by about 70%, by about 80%, by about 90%, or more) in the subject relative to a control (e.g., untreated subjects or placebo-treated subjects). The improvement in renal function in the subject may also include an improvement in creatinine clearance (e.g., by about 10%, by about 20%, by about 30%, by about 40%, but about 50%, by about 60%, by about 70%, by about 80%, by about 90%, or more) in the subject.

In some embodiments, said treating includes decreasing plasma Bb fragment of complement factor B (Bb) concentration (e.g., by about 10%, by about 20%, by about 30%, by about 40%, but about 50%, by about 60%, by about 70%, by about 80%, by about 90%, or more) and serum alternative pathway (AP) activity (e.g., by about 10%, by about 20%, by about 30%, by about 40%, but about 50%, by about 60%, by about 70%, by about 80%, by about 90%, or more), e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment.

In some embodiments, said treating includes reducing hematuria in the subject, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. Reducing hematuria in the subject may include a decrease in red blood cells (RBC) in urine from baseline (e.g., by about 10%, by about 20%, by about 30%, by about 40%, but about 50%, by about 60%, by about 70%, by about 80%, by about 90%, or more). Reducing hematuria may also include achieving <10 RBCs/hpf. In some embodiments, said treating includes reducing hematuria in the subject following a 50-week treatment period. In some embodiments, said treating includes reducing hematuria in the subject following a 26-week treatment period.

In some embodiments, treatment effect is measured by a 36-Item Short Form Survey Instrument (SF-36) score. The (SF-36) is a set of generic, coherent, and easily administered quality-of-life measures. It has 36 items grouped in 8 dimensions: Physical Functioning, Physical, Bodily Pain, Vitality, General Health, Emotion, Mental Health, and Social Functioning. An exemplary SF-36, scoring key, and scales are provided in Tables 1-3 below.

TABLE 1
36-Item Short Form Survey Instrument (SF-36)
General Health

| | | | | | |
|---|---|---|---|---|---|
| 1. In general, would you | Excellent (1) | Very good (2) | Good (3) | Fair (4) | Poor (5) |
| say your health is: | | | | | |
| 2. Compared to one year | Much better | Somewhat | About the | Somewhat | Much worse |
| ago, how would you rate | now than | better now | same (3) | worse now | now than |
| your health in general | one year | than one | | than one | one year |
| now? | ago (1) | year ago (2) | | year ago (4) | ago (5) |

Limitations of Activities:
The following items are about activities you might do during a typical day.
Does your health now limit you in these activities? If so, how much?

| | | | |
|---|---|---|---|
| 3. Vigorous activities, such as running, lifting | Yes, limited a | Yes, limited a | No, not limited |
| heavy objects, participating in strenuous sports | lot (1) | little (2) | at all (3) |
| 4. Moderate activities, such as moving a table, | Yes, limited a | Yes, limited a | No, not limited |
| pushing a vacuum cleaner, bowling, or playing golf | lot (1) | little (2) | at all (3) |
| | Yes, limited a | Yes, limited a | No, not limited |
| | lot (1) | little (2) | at all (3) |
| 5. Lifting or carrying groceries | Yes, limited a | Yes, limited a | No, not limited |
| | lot (1) | little (2) | at all (3) |
| 6. Climbing several flights of stairs | Yes, limited a | Yes, limited a | No, not limited |
| | lot (1) | little (2) | at all (3) |
| 7. Climbing one flight of stairs | Yes, limited a | Yes, limited a | No, not limited |
| | lot (1) | little (2) | at all (3) |
| 8. Bending, kneeling, or stopping | Yes, limited a | Yes, limited a | No, not limited |
| | lot (1) | little (2) | at all (3) |
| 9. Walking more than a mile | Yes, limited a | Yes, limited a | No, not limited |
| | lot (1) | little (2) | at all (3) |
| 10. Walking several blocks | Yes, limited a | Yes, limited a | No, not limited |
| | lot (1) | little (2) | at all (3) |
| 11. Walking one block | Yes, limited a | Yes, limited a | No, not limited |
| | lot (1) | little (2) | at all (3) |
| 12. Bathing or dressing yourself | Yes, limited a | Yes, limited a | No, not limited |
| | lot (1) | little (2) | at all (3) |

Physical Health Problems:
During the past 4 weeks, have you had any of the following problems with your
work or other regular daily activities as a result of your physical health?

| | | |
|---|---|---|
| 13. Cut down the amount of time you spent on work or other | Yes (1) | No (2) |
| activities | | |
| 14. Accomplished less than you would like | Yes (1) | No (2) |
| 15. Were limited in the kind of work or other activities | Yes (1) | No (2) |
| 16. Had difficulty performing the work on other activities (for | Yes (1) | No (2) |
| example, it took extra effort) | | |

Emotional Health Problems:
During the past 4 weeks, have you had any of the following problems with your work or other
regular daily activities as a result of any emotional problems (such as feeling depressed or anxious)?

| | | |
|---|---|---|
| 17. Cut down the amount of time you spent on work or other | Yes (1) | No (2) |
| activities | | |
| 18. Accomplished less than what you would like | Yes (1) | No (2) |
| 19. Didn't do work or other activities as carefully as usual | Yes (1) | No (2) |

Social Activities:

| | | | | | |
|---|---|---|---|---|---|
| 20. During the past 4 | Not at all | Slightly | Moderately | Quite a bit | Extremely |
| weeks, to what extent has | (1) | (2) | (3) | (4) | (5) |
| your physical health or | | | | | |
| emotional problems | | | | | |
| interfered with your normal | | | | | |
| social activities with family, | | | | | |
| friends, neighbors, or | | | | | |
| groups? | | | | | |

Pain:

| 21. How much bodily pain | None | Very mild | | Mild | | Moderate | | Severe | | Very |
|---|---|---|---|---|---|---|---|---|---|---|
| have you had during the | (1) | (2) | | (3) | | (4) | | (5) | | Severe |
| past 4 weeks? | | | | | | | | | | (6) |
| 22. During the past 4 | Not at all | | Slightly | | Moderately | | Quite a bit | | Extremely | |
| weeks, how much did pain | (1) | | (2) | | (3) | | (4) | | (5) | |
| interfere with your normal | | | | | | | | | | |
| work (including both work | | | | | | | | | | |
| outside the home and | | | | | | | | | | |
| housework)? | | | | | | | | | | |

Energy and Emotions:
These questions are about how you feel and how things have been with you during the past 4 weeks.
For each question, please give the one answer that comes closest to the way you have been feeling.
How much of the time during the past 4 weeks . . .

| | | | | | | |
|---|---|---|---|---|---|---|
| 23. Did you feel full of pep? | All of the | Most of | A good bit | Some of | A little of | None of |
| | time | the time | of the time | the time | the time | the time |
| | (1) | (2) | (3) | (4) | (5) | (6) |
| 24. Have you been a very | All of the | Most of | A good bit | Some of | A little of | None of |
| nervous person? | time | the time | of the time | the time | the time | the time |
| | (1) | (2) | (3) | (4) | (5) | (6) |
| 25. Have you felt so down | All of the | Most of | A good bit | Some of | A little of | None of |
| in the dumps that nothing | time | the time | of the time | the time | the time | the time |
| could cheer you up? | (1) | (2) | (3) | (4) | (5) | (6) |
| 26. Have you felt calm and | All of the | Most of | A good bit | Some of | A little of | None of |
| peaceful? | time | the time | of the time | the time | the time | the time |
| | (1) | (2) | (3) | (4) | (5) | (6) |
| 27. Did you have a lot of | All of the | Most of | A good bit | Some of | A little of | None of |
| energy? | time | the time | of the time | the time | the time | the time |
| | (1) | (2) | (3) | (4) | (5) | (6) |
| 28. Have you felt | All of the | Most of | A good bit | Some of | A little of | None of |
| downhearted and blue? | time | the time | of the time | the time | the time | the time |
| | (1) | (2) | (3) | (4) | (5) | (6) |
| 29. Did you feel worn out? | All of the | Most of | A good bit | Some of | A little of | None of |
| | time | the time | of the time | the time | the time | the time |
| | (1) | (2) | (3) | (4) | (5) | (6) |
| 30. Have you been a happy | All of the | Most of | A good bit | Some of | A little of | None of |
| person? | time | the time | of the time | the time | the time | the time |
| | (1) | (2) | (3) | (4) | (5) | (6) |
| 31. Did you feel tired? | All of the | Most of | A good bit | Some of | A little of | None of |
| | time | the time | of the time | the time | the time | the time |
| | (1) | (2) | (3) | (4) | (5) | (6) |

Social Activities:

| | | | | | |
|---|---|---|---|---|---|
| 32. During the past 4 | All of | Most of | Some of | A little | None of |
| weeks, how much of the | the time | the time | the time | of the time | the time |
| time has your physical | (1) | (2) | (3) | (4) | (5) |
| health or emotional | | | | | |
| problems interfered with | | | | | |
| your social activities (like | | | | | |
| visiting with friends, | | | | | |
| relatives, etc.)? | | | | | |

General Health:
How true or false is each of the following statements for you?

| | | | | | |
|---|---|---|---|---|---|
| 33. I seem to get sick a | Definitely | Mostly | Don't | Mostly | Definitely |
| little easier than other | true (1) | true (2) | know (3) | false (4) | false (5) |
| people. | | | | | |
| 34. I am as healthy as | Definitely | Mostly | Don't | Mostly | Definitely |
| anybody I know. | true (1) | true (2) | know (3) | false (4) | false (5) |
| 35. I expect my health to | Definitely | Mostly | Don't | Mostly | Definitely |
| get worse. | true (1) | true (2) | know (3) | false (4) | false (5) |
| 36. My health is excellent. | Definitely | Mostly | Don't | Mostly | Definitely |
| | true (1) | true (2) | know (3) | false (4) | false (5) |

TABLE 2
SF-36 Scoring Key

| Item numbers | Change original response category | To recoded value of: |
|---|---|---|
| 1, 2, 20, | 1 | 100 |
| 22, 34, 36 | 2 | 75 |
| | 3 | 50 |
| | 4 | 25 |
| | 5 | 0 |
| 3-12 | 1 | 0 |
| | 2 | 50 |
| | 3 | 100 |
| 13-19 | 1 | 0 |
| | 2 | 100 |
| 21, 23, 26, | 1 | 100 |
| 27, 30 | 2 | 80 |
| | 3 | 60 |
| | 4 | 40 |
| | 5 | 20 |
| | 6 | 0 |
| 24, 25, 28, | 1 | 0 |
| 29, 31 | 2 | 20 |
| | 3 | 40 |
| | 4 | 60 |
| | 5 | 80 |
| | 6 | 100 |
| 32, 33, 35 | 1 | 0 |
| | 2 | 25 |
| | 3 | 50 |
| | 4 | 75 |
| | 5 | 100 |

TABLE 3
Averaging Items to Form Scales

| | Number | After recoding per Table 2, |
|---|---|---|
| Scale | of items | average the following items |
| Physical functioning | 10 | 3-12 |
| Physical | 4 | 13-16 |
| Emotion | 3 | 17-19 |
| Vitality | 4 | 23, 27, 29, 31 |
| Mental health | 5 | 24-26, 28, 30 |
| Social functioning | 2 | 20, 32 |
| Bodily pain | 2 | 21, 22 |
| General health | 5 | 1, 33-36 |

In some embodiments, the treatment of LN or IgAN includes an improvement in a SF-36 score in one or more of physical functioning, physical, bodily pain, vitality, general health, emotion, mental health, and social functioning from baseline (e.g., by about 10%, by about 20%, by about 30%, by about 40%, but about 50%, by about 60%, by about 70%, by about 80%, by about 90%, or more), e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. The improvement may also be an increase in a SF-36 score in one or more mobility, usual activities, self-care, pain/discomfort, and anxiety/depression from baseline (e.g., by about 10%, by about 20%, by about 30%, by about 40%, but about 50%, by about 60%, by about 70%, by about 80%, by about 90%, or more), e.g., as compared to a control (such as an untreated or a placebo-treated subject). In some embodiments, the improvement in SF-36 score is observed after 8 weeks of treatment. In some embodiments, the improvement in SF-36 score is observed after 16 weeks of treatment. In some embodiments, the improvement in SF-36 score is observed after 26 weeks of treatment. In some embodiments, the improvement in SF-36 score is observed after 50 weeks of treatment.

In some embodiments, treatment effect is measured by a EuroQol 5-dimension 5-level questionnaire (EQ-5D-5L) score in one or more of mobility, usual activities, self-care, pain/discomfort, and anxiety/depression. The EuroQol 5-dimension 5-level questionnaire (EQ-5D-5L) is a standardized questionnaire for measuring health-related quality of life and is defined in 5 dimensions, i.e., mobility, usual activities, self-care, pain/discomfort, and anxiety/depression. A representative method for EQ-5D-5L is based on 0 to 100 health state visual analog scale (VAS) accompanying the above 5 dimensions, where 0 indicates worst health and 100 best health. A 0 to 1 index or utility score is calculated from the 5 dimensions using a preference-based value set, where 0 indicates a health state equivalent to death and 1 indicates perfect health. Negative values indicate health states considered worse than death. Each of the EQ-5D-5L dimensions may be summarized and analyzed as a categorical variable, providing data on the health profile of the study patients. The VAS and EQ-5D-5L index score may be summarized and analyzed as continuous variables. An exemplary EQ-5D-5L questionnaire is provided in Table 4 below.

TABLE 4
EuroQol 5-Dimension 5-Level Questionnaire (EQ-5D-5L)
MOBILITY

| | |
|---|---|
| I have no problems in walking about | 1 |
| I have no slight problems in walking about | 2 |
| I have moderate problems in walking about | 3 |
| I have severe problems in walking about | 4 |
| I am unable to walk about | 5 |

SELF-CARE

| | |
|---|---|
| I have no problems washing or dressing myself | 1 |
| I have slight problems washing or dressing myself | 2 |
| I have moderate problems washing or dressing | 3 |
| myself | |
| I have severe problems washing or dressing | 4 |
| myself | |
| I am unable to wash or dress myself | 5 |

USUAL ACTIVITIES (e.g., work, study,
housework, family or leisure activities)

| | |
|---|---|
| I have no problems doing my usual activities | 1 |
| I have slight problems doing my usual activities | 2 |
| I have moderate problems doing my usual | 3 |
| activities | |
| I have severe problems doing my usual activities | 4 |
| I am unable to do my usual activities | 5 |

PAIN/DISCOMFORT

| | |
|---|---|
| I have no pain or discomfort | 1 |
| I have slight pain or discomfort | 2 |
| I have moderate pain or discomfort | 3 |
| I have severe pain or discomfort | 4 |
| I have extreme pain or discomfort | 5 |

ANXIETY/DEPRESSION

| | |
|---|---|
| I am not anxious or depressed | 1 |
| I am slightly anxious or depressed | 2 |
| I am moderately anxious or depressed | 3 |
| I am severely anxious or depressed | 4 |
| I am extremely anxious or depressed | 5 |

In some embodiments, the treatment of LN and/or IgAN includes an improvement in an EQ-5D-5L score in one or more of mobility, usual activities, self-care, pain/discomfort, and anxiety/depression from baseline (e.g., by about 10%, by about 20%, by about 30%, by about 40%, but about 50%, by about 60%, by about 70%, by about 80%, by about 90%, or more), e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. The improvement may be an increase in an EQ-5D-5L score in one or more mobility, usual activities, self-care, pain/discomfort, and anxiety/depression from baseline, e.g., as compared to a control (such as an untreated or a placebo-treated subject). In some embodiments, the improvement in EQ-5D-5L score is observed after 8 weeks of treatment. In some embodiments, the improvement in EQ-5D-5L score is observed after 16 weeks of treatment. In some embodiments, the improvement in EQ-5D-5L score is observed after 26 weeks of treatment. In some embodiments, the improvement in EQ-5D-5L score is observed after 50 weeks of treatment.

In some embodiments, the subject has an eGFR <30 mL/min/1.73 m² as calculated using CKD-EPI.

In some embodiments, the subject has less than 50% tubular atrophy, glomerular sclerosis, or crescent formation in glomeruli on the most recent kidney biopsy obtained prior to treatment.

In some embodiments, the subject does not have a concomitant significant renal disease other than LN or IgAN on the most recent kidney biopsy obtained prior to treatment with Compound 1 or the pharmaceutically acceptable salt thereof.

In some embodiments, the subject does not have a history of solid organ or bone marrow transplant.

In some embodiments, the subject is restricted from receiving a solid organ or bone marrow transplant during a 50-week treatment period.

In some embodiments, the subject has not had a splenectomy and does not have functional asplenia.

In some embodiments, the subject does not have a history of seizure.

In some embodiments, the subject does not have a known or suspected complement deficiency unless the complement deficiency is attributable to LN or IgAN.

In some embodiments, the subject does not have a history of or have risk factors for Torsades de Pointes, a screening QT interval corrected using Fridericia's formula (QTcF)>450 msec when the subject is male or >470 msec when the subject is female or is receiving medication known to significantly increase the corrected QT interval (QTc). Examples of such medications include, but are not limited to, typical (1^{st} generation) antipsycholtics (e.g., thiordazine, haloperidol, chlorpromazine, pimozide, or loxapine), atypical (2^{nd} generation) antipsychotics (e.g., ziprasidone, iloperidone, quetiapine, olanzapine, risperidone, paliperidone, aripiprazole, asenapine, clozapine, brexipipirazole, or purasidone), selective serotonin reuptake inhibitors (SSRIs; e.g., citalopram, escitalopram, paroxetine, fluoxetine, sertraline, or fluvoxamine), tricyclic or tetracyclic antidepressants (TCAs or TeCAs; e.g., amitriptyline, imipramine, maprotiline, nortriptyline, desipramine, clomipramine, trimipramine, or doxepin), serotonin-norepinephrine reuptake inhibitors (SNRIs; e.g., venlafaxine, duloxetine, desvenlafaxine, levomilnacipran, or milnacipran), and other antidepressants (e.g., mirtazapine, bupropion, vortioxetine, vilazodone, or trazodone).

In some embodiments, the subject does not have an alanine aminotransferase level of >2× upper limit of normal (ULN).

In some embodiments, the subject does not have a direct bilirubin level of >2×ULN.

In some embodiments, the subject has a hemoglobin A1C level of ≤7.0% prior to treatment with Compound 1 or the pharmaceutically acceptable salt thereof.

In some embodiments, the subject does not have a known or suspected history of drug or alcohol abuse or dependence within 1 year prior to treatment.

In some embodiments, the subject does not have a history of malignancy within 5 years prior to treatment, wherein the malignancy is not nonmelanoma skin cancer or carcinoma in situ of the cervix that has been treated with no evidence of recurrence.

In some embodiments, the subject is not exhibiting signs of a hepatitis B viral infection with negative surface antibodies.

In some embodiments, the subject is not exhibiting signs of a hepatitis C viral infection; or is exhibiting signs of a hepatitis C viral infection but has been successfully treated and has a documented sustained virologic response.

In some embodiments, the subject is not exhibiting signs of a human immunodeficiency virus infection.

In some embodiments, the subject does not have bone marrow insufficiency or thrombocytopenia.

In some embodiments, the subject did not have an active systemic bacterial, viral or fungal infection within 14 days prior to treatment.

In some embodiments, the subject does not have a history of N meningitidis infection.

In some embodiments, the subject is not receiving treatment with a biologic medication that may affect immune system functioning (e.g., a corticosteroid or an immunosuppressant); or has stopped receiving treatment with the biologic medication, and 5 terminal half-lives of the biologic has elapsed prior to treatment with Compound 1 or the pharmaceutically acceptable salt thereof.

In some embodiments, the subject has not received treatment with belimumab or rituximab ≤6 months prior to treatment with Compound 1 or the pharmaceutically acceptable salt thereof.

In some embodiments, the subject has not received, or is not receiving treatment with a complement inhibitor other than Compound 1 or the pharmaceutically acceptable salt thereof.

In some embodiments, the subject has not received treatment with a medication selected from a strong cytochrome P450, family 3, subfamily A (CYP3A) inhibitor (e.g., boceprevir; cobicistat; danoprevir and ritonavir; elvitegravir and ritonavir; grapefruit juice; indinavir and ritonavir; itraconazole; ketoconazole; lopinavir and ritonavir; paritaprevir, ritonavir, and ombitasvir and/or dasabuvir; Posaconazole; ritonavir; saquinavir and ritonavir; telaprevir; tipranavir and ritonavir; telithromycin; troleandomycin; voriconazole; clarithromycin; idelalisib; nefazodone; or nelfinavir); a moderate CYP3A inhibitor (e.g., aprepitant, ciprofloxacin, conivaptan, crizotinib, cyclosporine, diltiazem, dronedarone, erythromycin, fluconazole, fluvoxamine, imatinib, tofisopam, or verapamil); a strong inducer of CYP3A (e.g., apalutamide, carbamazepine, enzalutamide, mitotane, phenytoin, rifampin, or St. John's wort); a moderate inducer of CYP3A (e.g., bosentan, efavirenz, etravirine, phenobarbital, or primidone); and a sensitive substrate of CYP3A (e.g., alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, or tolvaptan), within the longer of two weeks or five half-lives of the medication prior to the treatment with Compound 1 or the pharmaceutically acceptable salt thereof.

In some embodiments, the subject has not received treatment with a medication known to lower the seizure threshold and/or cause seizure. Examples of such medications include, but are not limited to, meperidine, pethidine, a typical (1^{st} generation) antipsychotic, clozapine, olanzapine, lithium, a tricyclic antidepressant, bupropion, aminophylline, and theophylline.

In some embodiments, the subject is not pregnant or breastfeeding.

In some embodiments, the subject is restricted from consuming foods and beverages that inhibit CYP3A4 enzyme activity.

In some embodiments, the subject is restricted from receiving treatment with a complement inhibitor other than Compound 1 or a pharmaceutically acceptable salt thereof (e.g., an anti-C5 antibody such as eculizumab or ravulizumab).

In some embodiments, the subject has been receiving treatment with a sodium-glucose cotransporter-2 (SGLT-2) inhibitor on a stable dose (e.g., for 3 months) prior to treatment with Compound 1 or the pharmaceutically acceptable salt thereof, and the dose of the SGLT-2 inhibitor does not change during treatment (e.g., through a 50-week treatment period) with Compound 1 or the pharmaceutically acceptable salt thereof.

In some embodiments, the subject has not been receiving treatment with a SGLT-2 inhibitor prior to treatment with Compound 1 or the pharmaceutically acceptable salt thereof, and the subject is restricted from receiving treatment with a SGLT-2 inhibitor within a 50-week period following the start of treatment with Compound 1 or the pharmaceutically acceptable salt thereof.

In some embodiments, the subject is restricted from using a medication selected from a strong cytochrome P450, family 3, subfamily A (CYP3A) inhibitor (e.g., boceprevir; cobicistat; danoprevir and ritonavir; elvitegravir and ritonavir; grapefruit juice; indinavir and ritonavir; itraconazole; ketoconazole; lopinavir and ritonavir; paritaprevir, ritonavir, and ombitasvir and/or dasabuvir; Posaconazole; ritonavir; saquinavir and ritonavir; telaprevir; tipranavir and ritonavir; telithromycin; troleandomycin; voriconazole; clarithromycin; idelalisib; nefazodone; or nelfinavir); a moderate CYP3A inhibitor (e.g., aprepitant, ciprofloxacin, conivaptan, crizotinib, cyclosporine, diltiazem, dronedarone, erythromycin, fluconazole, fluvoxamine, imatinib, tofisopam, or verapamil); a strong inducer of CYP3A (e.g., apalutamide, carbamazepine, enzalutamide, mitotane, phenytoin, rifampin, or St. John's wort); a moderate inducer of CYP3A (e.g., bosentan, efavirenz, etravirine, phenobarbital, or primidone); and a sensitive substrate of CYP3A (e.g., alfentanil, avanafil, buspirone, conivaptan, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, or tolvaptan).

In some embodiments, the subject is restricted from using a medication known to lower the seizure threshold and/or cause seizure. Examples of such medications include, but are not limited to, meperidine, pethidine, a typical (1^{st} generation) antipsychotic, clozapine, olanzapine, lithium, a tricyclic antidepressant, bupropion, aminophylline, and theophylline.

In some embodiments, the subject is restricted from using a medication known to significantly prolong QTc, provided that the medication is not hydroxychloroquine for use by a subject with LN. Examples of such medications include, but are not limited to, typical (1^{st} generation) antipsycholtics (e.g., thiordazine, haloperidol, chlorpromazine, pimozide, or loxapine), atypical (2^{nd} generation) antipsychotics (e.g., ziprasidone, iloperidone, quetiapine, olanzapine, risperidone, paliperidone, aripiprazole, asenapine, clozapine, brexipipirazole, or purasidone), selective serotonin reuptake inhibitors (SSRIs; e.g., citalopram, escitalopram, paroxetine, fluoxetine, sertraline, or fluvoxamine), tricyclic or tetracyclic antidepressants (TCAs or TeCAs; e.g., amitriptyline, imipramine, maprotiline, nortriptyline, desipramine, clomipramine, trimipramine, or doxepin), serotonin-norepinephrine reuptake inhibitors (SNRIs; e.g., venlafaxine, duloxetine, desvenlafaxine, levomilnacipran, or milnacipran), and other antidepressants (e.g., mirtazapine, bupropion, vortioxetine, vilazodone, or trazodone).

### Treatment of LN

In some embodiments, the subject has LN (e.g., de novo LN or relapsing LN).

In some embodiments, the subject has been diagnosed with active focal or diffuse proliferative LN class II or IV confirmed by a kidney biopsy obtained <6 months prior to treatment. The subject may also be exhibiting Class V disease.

In some embodiments, the LN is clinically active LN which requires immunosuppression induction treatment.

In some embodiments, the subject has proteinuria with urine protein:creatinine ratio (UPCR) 1 g/g based on a 24-hour urine collection prior to treatment.

In some embodiments, in which the subject has LN, the time to first occurrence of UPCR<0.5 g/g as measured by spot urine sample is reduced as compared to a control (e.g., untreated subjects or placebo-treated subjects).

In some embodiments, the subject experiences partial renal response (PRR; as defined by the criteria set forth in Example 1), e.g., after 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. In some embodiments, the subject experiences PRR following a 26-week treatment period. In some embodiments, the subject experiences PRR following a 50-week treatment period.

In some embodiments, the subject experiences complete renal response (CRR; as defined by the criteria set forth in Example 1), e.g., after 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. In some embodiments, the subject experiences CRR following a 26-week treatment period. In some embodiments, the subject experiences CRR following a 50-week treatment period.

In some embodiments, said treating comprises reducing a time to first occurrence of UPCR<0.5 g/g from baseline as measured by spot urine sample (e.g., by 1, 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks), as compared to a control (e.g., untreated subjects or placebo-treated subjects).

In some embodiments, the subject has not started corticosteroid induction treatment prior to (e.g., within a period of up to 6 weeks prior to) treatment with Compound 1 or the pharmaceutically acceptable salt thereof.

In some embodiments, in which the subject has not started corticosteroid induction treatment within prior to (e.g., within a period of up to 6 weeks prior to) treatment with Compound 1 or the pharmaceutically acceptable salt thereof, the subject may be administered a cumulative dose of about 1 g of methylprednisolone IV in one or multiple divided doses within a period of up to 6 weeks prior to the start of treatment with Compound 1 or the pharmaceutically acceptable salt thereof.

In some embodiments, in which the subject has not started corticosteroid induction treatment prior to (e.g., within a period of up to 6 weeks prior to) treatment with Compound 1 or the pharmaceutically acceptable salt thereof, the subject has been administered an oral corticosteroid at a dose of about 0.5 mg/kg/day with a minimum dose of about 30 mg/day and a maximum dose of about 60 mg/day for a period of up to six weeks prior to the start of treatment with Compound 1 or the pharmaceutically acceptable salt thereof.

In some embodiments, in which the subject has not started corticosteroid induction treatment prior to (e.g., within a period of up to 6 weeks prior to) treatment with Compound 1 or the pharmaceutically acceptable salt thereof, said treating comprises co-administering an oral corticosteroid at a dose of about 30 mg/day to about 60 mg/day. The dose of the oral corticosteroid may be tapered to about 7.5 mg/day, e.g., after a 12-week, 26-week, or 50-week treatment period.

In some embodiments, in which the subject has not started corticosteroid induction treatment prior to (e.g., within a period of up to 6 weeks prior to) treatment with Compound 1 or the pharmaceutically acceptable salt thereof, the subject is administered a first dose of mycophenolate mofetil (MMF) about 1-1.5 g/day (or an equivalent dose of enteric-coated mycophenolic acid sodium (MPS)) in one or more doses after administration of the cumulative dose of about 1 g of methylprednisolone IV prior to (e.g., up to a period of 6 weeks prior to) treatment with Compound 1 or the pharmaceutically acceptable salt thereof, and is administered a second dose of MMF (or an equivalent dose of enteric-coated MPS) in one or more doses until 50 weeks after the start of treatment with Compound 1 or the pharmaceutically acceptable salt thereof. In some embodiments, the second dose is about 1-1.5 g/day. In some embodiments, the second dose is about 1-1.5 g/day until 1 week after the start of treatment with Compound 1 or the pharmaceutically acceptable salt thereof, after which the second dose is increased to about 2-3 g/day.

In some embodiments, the subject has initiated corticosteroid induction treatment prior to (e.g., up to a period 6 weeks prior to) treatment with Compound 1 or the pharmaceutically acceptable salt thereof.

In some embodiments, in which the subject has initiated corticosteroid induction treatment prior to (e.g., up to a period of 6 weeks prior to) treatment with Compound 1 or the pharmaceutically acceptable salt thereof, the subject has received a cumulative dose of methylprednisolone IV of 1 g or an equivalent oral corticosteroid and is receiving a first dose of MMF of about 2 g/day (or an equivalent dose of enteric-coated MPS) in one or more doses prior to (e.g., up to a period of 6 weeks prior to) treatment with Compound 1 or the pharmaceutically acceptable salt thereof, the subject is not administered an additional dose of methylprednisolone IV or equivalent oral corticosteroid, and the subject is administered a second dose of MMF (or an equivalent dose of enteric-coated MPS) in one or more doses until 50 weeks after the start of treatment with Compound 1 or the pharmaceutically acceptable salt thereof. In some embodiments, the second dose of MMF is about 2 g/day. In some embodiments, the second dose of MMF is about 2 g/day and is adjusted to about 2-3 g/day before 4 weeks after the start of treatment with Compound 1 or the pharmaceutically acceptable salt thereof.

In some embodiments, in which the subject has initiated corticosteroid induction treatment prior to (e.g., up to a period of 6 weeks prior to) treatment with Compound 1 or the pharmaceutically acceptable salt thereof, the subject has received a cumulative dose of methylprednisolone IV of about 1 g or an equivalent oral corticosteroid and is receiving a first dose of MMF of <about 2 g/day (or an equivalent dose of enteric-coated MPS) in one or more doses prior to (e.g., up to a period of 6 weeks prior to) treatment with Compound 1 or the pharmaceutically acceptable salt thereof, and the subject is not administered an additional dose of methylprednisolone IV or equivalent oral corticosteroid, and the subject is administered a second dose of MMF (or an equivalent dose of enteric-coated MPS) in one or more doses until 50 weeks after the start of treatment with Compound 1 or the pharmaceutically acceptable salt thereof. In some embodiments, the second dose of MMF is about 1-1.5 g/day at the start of treatment with Compound 1 or the pharmaceutically acceptable salt thereof. In some embodiments, the second dose of MMF is about 1-1.5 g/day for a one-week period following the start of treatment with Compound 1 or the pharmaceutically acceptable salt thereof, after which the second dose of MMF is increased to about 2-3 g/day before 4 weeks after the start of treatment with Compound 1 or the pharmaceutically acceptable salt thereof.

In some embodiments, in which the subject has initiated corticosteroid induction treatment prior to (e.g., up to a period of 6 weeks prior to) treatment with Compound 1 or the pharmaceutically acceptable salt thereof, the subject has been receiving prednisone or a prednisone equivalent at a first dose prior to the start of treatment with Compound 1 or the pharmaceutically acceptable salt thereof, the first dose is maintained through the second day of treatment with Compound 1 or the pharmaceutically acceptable salt thereof, after which the subject is administered an oral corticosteroid (e.g., prednisone or a prednisone equivalent) at a dose of about 0.5 mg/kg/day with a minimum dose of about 30 mg/day and a maximum dose of about 60 mg/day. The dose of the oral corticosteroid may be tapered to about 7.5 mg/day, e.g., after a 12-week, 26-week, or 50-week treatment period.

In some embodiments, said treating comprises reducing a risk of experiencing a renal flare in the subject, e.g., within a 50-week treatment period.

In some embodiments, said treating comprises reducing a risk of experiencing an extrarenal systemic lupus erythematosus (SLE) flare in the subject, e.g., within a 50-week treatment period.

In some embodiments, said treating comprises reducing a risk of treatment failure in the subject, e.g., within a 50-week treatment period.

In some embodiments, said treating comprises reducing a risk of suboptimal response in the subject, e.g., within a 50-week treatment period.

In some embodiments, said treating comprises reducing a level of serum albumin in the subject from baseline.

In some embodiments, said treating comprises reducing the time to first CRR or PRR (e.g., as determined by spot UCPR) as compared to a control (e.g., untreated subjects or placebo-treated subjects).

In some embodiments, said treating comprises reducing the time to first occurrence of UPCR>50% decrease from baseline (e.g., as determined by spot UCPR) as compared to a control (e.g., untreated subjects or placebo-treated subjects).

In some embodiments, the subject exhibits an increase in The Functional Assessment of Chronic Illness Therapy (FACIT) Fatigue Scale total score from baseline, e.g., following a 26-week or 50-week treatment period. The FACIT Fatigue Scale is a 40-item measure that assess self-reported fatigue and its impact upon daily activities and function. An exemplary FACIT Fatigue and the corresponding scoring guide are shown in Tables 5 and 6 below.

TABLE 5
FACIT Fatigue Questionnaire

Below is a list of statements that other people with your illness have said are important. Please
circle or mark one number per line to indicate your response as it applies to the past 7 days.

| | | Not at all | A little bit | Somewhat | Quite a bit | Very much |
|---|---|---|---|---|---|---|
| Physical Well-Being | | | | | | |
| GP1 | I have a lack of | 0 | 1 | 2 | 3 | 4 |
| | energy. | | | | | |
| GP2 | I have nausea. | 0 | 1 | 2 | 3 | 4 |
| GP3 | Because of my | 0 | 1 | 2 | 3 | 4 |
| | physical condition, I | | | | | |
| | have trouble | | | | | |
| | meeting the needs | | | | | |
| | of my family. | | | | | |
| GP4 | I have pain. | 0 | 1 | 2 | 3 | 4 |
| GP5 | I am bothered by | 0 | 1 | 2 | 3 | 4 |
| | side effects of | | | | | |
| | treatment. | | | | | |
| GP6 | I feel ill. | 0 | 1 | 2 | 3 | 4 |
| GP7 | I am forced to spend | 0 | 1 | 2 | 3 | 4 |
| | time in bed. | | | | | |
| Social/Family Well-Being | | | | | | |
| GS1 | I feel close to my | 0 | 1 | 2 | 3 | 4 |
| | friends. | | | | | |
| GS2 | I get emotional | 0 | 1 | 2 | 3 | 4 |
| | support from my | | | | | |
| | family. | | | | | |
| GS3 | I get support from | 0 | 1 | 2 | 3 | 4 |
| | my friends. | | | | | |
| GS4 | My family has | 0 | 1 | 2 | 3 | 4 |
| | accepted my illness. | | | | | |
| GS5 | I am satisfied with | 0 | 1 | 2 | 3 | 4 |
| | family communication | | | | | |
| | about my illness | | | | | |
| GS6 | I feel close to my | 0 | 1 | 2 | 3 | 4 |
| | partner (or the | | | | | |
| | person who is my | | | | | |
| | main support). | | | | | |

| | |
|---|---|
| Q1 | Regardless of your current level of sexual activity, please answer the following question. |
| | If you prefer not to answer it, please mark this box and go to the next section. |

| | | | | | | |
|---|---|---|---|---|---|---|
| GS7 | I am satisfied with | 0 | 1 | 2 | 3 | 4 |
| | my sex life. | | | | | |

Please circle or mark one number per line to indicate your response as it applies to the past 7 days.

| | | Not at all | A little bit | Somewhat | Quite a bit | Very much |
|---|---|---|---|---|---|---|
| Emotional Well-Being | | | | | | |
| GE1 | I feel sad. | 0 | 1 | 2 | 3 | 4 |
| GE2 | I am satisfied with | 0 | 1 | 2 | 3 | 4 |
| | how I am coping | | | | | |
| | with my illness. | | | | | |
| GE3 | I am losing hope in | 0 | 1 | 2 | 3 | 4 |
| | the fight against my | | | | | |
| | illness. | | | | | |
| GE4 | I feel nervous. | 0 | 1 | 2 | 3 | 4 |
| GE5 | I worry about dying. | 0 | 1 | 2 | 3 | 4 |
| GE6 | I worry that my | 0 | 1 | 2 | 3 | 4 |
| | condition will get | | | | | |
| | worse. | | | | | |
| Function Well-Being | | | | | | |
| GF1 | I am able to work | 0 | 1 | 2 | 3 | 4 |
| | (including work at | | | | | |
| | home). | | | | | |
| GF2 | My work (include | 0 | 1 | 2 | 3 | 4 |
| | work at home) is | | | | | |
| | fulfilling. | | | | | |
| GF3 | I am able to enjoy | 0 | 1 | 2 | 3 | 4 |
| | life. | | | | | |
| GF4 | I have accepted my | 0 | 1 | 2 | 3 | 4 |
| | illness. | | | | | |
| GF5 | I am sleeping well. | 0 | 1 | 2 | 3 | 4 |
| GF6 | I am enjoying the | 0 | 1 | 2 | 3 | 4 |
| | things I usually do | | | | | |
| | for fun. | | | | | |
| GF7 | I am content with | 0 | 1 | 2 | 3 | 4 |
| | the quality of my life | | | | | |
| | right now. | | | | | |
| Additional Concerns | | | | | | |
| HI7 | I feel fatigued. | 0 | 1 | 2 | 3 | 4 |
| HI12 | I feel weak all over. | 0 | 1 | 2 | 3 | 4 |
| An1 | I feel listless | 0 | 1 | 2 | 3 | 4 |
| | (“washed out”). | | | | | |
| An2 | I feel tired. | 0 | 1 | 2 | 3 | 4 |
| An3 | I have trouble | 0 | 1 | 2 | 3 | 4 |
| | starting things | | | | | |
| | because I am tired. | | | | | |
| An4 | I have trouble | 0 | 1 | 2 | 3 | 4 |
| | finishing things | | | | | |
| | because I am tired. | | | | | |
| An5 | I have energy. | 0 | 1 | 2 | 3 | 4 |
| An7 | I am able to do my | 0 | 1 | 2 | 3 | 4 |
| | usual activities. | | | | | |
| An8 | I need to sleep | 0 | 1 | 2 | 3 | 4 |
| | during the day. | | | | | |
| An12 | I am too tired to eat. | 0 | 1 | 2 | 3 | 4 |
| An14 | I need help doing | 0 | 1 | 2 | 3 | 4 |
| | my usual activities. | | | | | |
| An15 | I am frustrated by | 0 | 1 | 2 | 3 | 4 |
| | being too tired to do | | | | | |
| | the things I want to | | | | | |
| | do. | | | | | |
| An16 | I have to limit my | 0 | 1 | 2 | 3 | 4 |
| | social activity | | | | | |
| | because I am tired. | | | | | |

TABLE 6
FACIT Fatigue Questionnaire Scoring Guidelines
Instructions:
1. Record answers in “item response” column. If missing, mark with an X.
2. Perform reversals as indicated, and
sum individual items to obtain a score.
3. Multiply the sum of the item scores by the number
of items in the subscale, then divide by the number
item answered. This produces the subscale score.
4. Add subscale scores to derive total scores.
5. The higher the score, the better then QOL.

| | Item | Reverse | Item | Item |
|---|---|---|---|---|
| Subscale | Code | item? | Response | Score |
| PHYSICAL | GP1 | 4 − | | = |
| WELL-BEING | GP2 | 4 − | | = |
| (PWB) | GP3 | 4 − | | = |
| Score range: | GP4 | 4 − | | = |
| 0-28 | GP5 | 4 − | | = |
| | GP6 | 4 − | | = |
| | GP7 | 4 − | | = |
| | Sum individual item scores: | | | |
| | Multiply by 7: | | | |
| | Divide by number of items answered | | | |
| | for PWB subscale score: | | | |
| SOCIAL/ | GS1 | 0 + | | = |
| FAMILY | GS2 | 0 + | | = |
| WELL-BEING | GS3 | 0 + | | = |
| (SWB) | GS4 | 0 + | | = |
| Score range: | GS5 | 0 + | | = |
| 0-28 | GS6 | 0 + | | = |
| | GS7 | 0 + | | = |
| | Sum individual item scores: | | | |
| | Multiply by 7: | | | |
| | Divide by number of items answered | | | |
| | for SWB subscale score: | | | |
| EMOTIONAL | GE1 | 4 − | | = |
| WELL-BEING | GE2 | 0 + | | = |
| (EWB) | GE3 | 4 − | | = |
| Score range: | GE4 | 4 − | | = |
| 0-28 | GE5 | 4 − | | = |
| | GE6 | 4 − | | = |
| | Sum individual item scores: | | | |
| | Multiply by 6: | | | |
| | Divide by number of items answered | | | |
| | for EWB subscale score: | | | |
| FUNCTIONAL | GS1 | 0 + | | = |
| WELL-BEING | GS2 | 0 + | | = |
| (FWB) | GS3 | 0 + | | = |
| Score range: | GS4 | 0 + | | = |
| 0-28 | GS5 | 0 + | | = |
| | GS6 | 0 + | | = |
| | GS7 | 0 + | | = |
| | Sum individual item scores: | | | |
| | Multiply by 7: | | | |
| | Divide by number of items answered | | | |
| | for FWB subscale score: | | | |
| FATIGUE | HI7 | 4 − | | = |
| SUBSCALE | HI12 | 4 − | | = |
| (FS) | An1 | 4 − | | = |
| Score range: | An2 | 4 − | | = |
| 0-28 | An3 | 4 − | | = |
| | An4 | 4 − | | = |
| | An5 | 0 + | | = |
| | An7 | 0 + | | = |
| | An8 | 4 − | | = |
| | An12 | 4 − | | = |
| | An14 | 4 − | | = |
| | An15 | 4 − | | = |
| | An16 | 4 − | | = |

| | Sum individual item scores: | | | | | |
|---|---|---|---|---|---|---|
| | Multiply by 13: | | | | | |
| | Divide by number of items answered | | | | | |
| | for FS subscale score: | | | | | |
| To Derive a | + | + | + | + | + | = |
| FACIT Fatigue | (PWB | (SWB | (EWB | (FWB | (FS | FACIT |
| total score: | score) | score) | score) | score) | score) | Fatigue |
| Score range: | | | | | | Total |
| 0-160 | | | | | | score |

In some embodiments, the treatment of LN includes an increase in FACIT Fatigue total score exhibited by the subject from baseline. In some embodiments, the subject exhibits an increase in FACIT Fatigue total score of at least 10 points (e.g., 10, 11, 12, 13 points or more) from baseline, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. In some embodiments, the treatment results in an increase in FACIT fatigue score by at least about 25% (e.g., at least about 30%, at least about 35%, at least about 40%, at least about 45%, at least about 50%, at least about 55%, at least about 60%, at least about 65%, at least about 70%, at least about 80%, at least about 90%, at least about 95%, at least about 100%, at least about 120%, at least about 140%, at least about 150%, at least about 160%, at least about 180%, at least about 200%, at least about 250%, at least about 300%, at least about 350%, or at least about 400%) compared to baseline, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. In some embodiments, the subject exhibits an increase in FACIT Fatigue total score of at least 10 points (e.g., 10, 11, 12, 13 points or more) after 8 weeks of treatment. In some embodiments, the subject exhibits an increase in FACIT Fatigue total score of at least 10 points (e.g., 10, 11, 12, 13 points or more) after 16 weeks of treatment. In some embodiments, the subject exhibits an increase in FACIT Fatigue total score of at least 10 points (e.g., 10, 11, 12, 13 points or more) after 26 weeks of treatment. In some embodiments, the subject exhibits an increase in FACIT Fatigue total score of at least 10 points (e.g., 10, 11, 12, 13 points or more) after 50 weeks of treatment.

In some embodiments, a level of antibodies against double-stranded DNA (anti-dsDNA) and/or antibodies against C1q complement component (anti-C1q) in the subject is reduced from baseline, e.g., after 2, 3, 4, 5, 6, 7, 8, 9, 10, 11, 12, 13, 14, 15, 16, 17, 18, 19, 20, 21, 22, 23, 24, 25, 26, 27, 28, 29, 20, 31, 32, 36, 40, 44, 48, 52, 56, 60, 64, 68, 72, 76, 70, 84, 88, 92, 96, 102, 104, 108, or 154 weeks of treatment. In some embodiments, the level of anti-dsDNA and/or anti-C1q is in the subject is reduced from baseline after 26 weeks of treatment. In some embodiments, the level of anti-dsDNA and/or anti-C1q is in the subject is reduced from baseline after 50 weeks of treatment.

In some embodiments, the subject has not received treatment with cyclophosphamide ≤6 months prior to treatment with Compound 1 or the pharmaceutically acceptable salt thereof.

In some embodiments, the subject has not received treatment with a calcineurin inhibitor ≤3 months prior to treatment with Compound 1 or the pharmaceutically acceptable salt thereof.

In some embodiments, the subject has not received treatment with a cumulative dose of intravenous (IV) methylprednisolone >about 3 g for active renal flare.

In some embodiments, the subject has not received treatment with MMF >about 2 g/day (or an equivalent thereof, e.g., MPS) for ≥4 consecutive weeks for active renal flare prior to treatment of Compound 1 or the pharmaceutically acceptable salt thereof.

In some embodiments, the subject has not received treatment with prednisone about 0.5 mg/kg/day or an equivalent thereof for 4 consecutive weeks for active renal flare prior to treatment of Compound 1 or the pharmaceutically acceptable salt thereof.

In some embodiments, the subject does not have uncontrolled hypertension (systolic blood pressure >160 mmHg or diastolic blood pressure >110 mmHg) on 2 or more measurements within a 6-week period prior to treatment with Compound 1 or the pharmaceutically acceptable salt thereof.

In some embodiments, the subject does not have a history of or has clinically active SLE-related cerebritis, seizures, pericarditis, stroke, or stroke syndrome requiring treatment.

In some embodiments, the subject does not have an inability to take or tolerate treatment with a corticosteroid, MMF, or MPS.

In some embodiments, the subject is restricted from receiving treatment with a calcineurin inhibitor (e.g., voclosporin).

### Treatment of IgAN

In some embodiments, the subject has IgAN.

In some embodiments, the IgAN is primary IgAN confirmed by a kidney biopsy obtained prior to treatment (see Table 15 for the IgAN classification criteria). In some embodiments, the kidney is obtained more than 2 years prior to treatment, and the subject has hematuria as defined by 1+ blood based on urine dipstick or ≥10 red blood cells (RBCs)/high-power field (hpf) microscopy on urine sediment.

In some embodiments, the subject has been receiving treatment with a stable and optimal dose of a RAS inhibitor (e.g., maximum allowed or tolerated angiotensin-converting enzyme (ACE) inhibitor and/or angiotensin receptor blocker (ARB) dose) prior to treatment with Compound 1 or the pharmaceutically acceptable salt thereof and continues to receive treatment with the RAS inhibitor during treatment with Compound 1 or the pharmaceutically acceptable salt thereof. In some embodiments, the subject is restricted from receiving treatment with a second RAS inhibitor (e.g., an ACE inhibitor and/or an ARB) within a 50-week period following the start of treatment with Compound 1 or the pharmaceutically acceptable salt thereof.

In some embodiments, the subject has been receiving treatment with a stable and optimal dose of a direct renin antagonist prior to treatment with Compound 1 or the pharmaceutically acceptable salt thereof and continues to receive treatment with the direct renin antagonist during treatment with Compound 1 or the pharmaceutically acceptable salt thereof. In some embodiments, the subject is restricted from receiving treatment with a second direct renin antagonist within a 50-week period following the start of treatment with Compound 1 or the pharmaceutically acceptable salt thereof.

In some embodiments, the subject has controlled and stable blood pressure (i.e., <140/90 mmHg) over a 3-month period prior to treatment with Compound 1 or the pharmaceutically acceptable salt thereof.

In some embodiments, the subject experiences partial remission, e.g., following a 26-week or 50-week treatment period.

In some embodiments, said treating includes attenuating or flattening the slope of eGFR computed from baseline to, e.g., week 26 and/or week 50 of a treatment period as compared to a control (e.g., untreated subjects or placebo-treated subjects). See, e.g., Barratt et al., Kidney Int Rep. 2019; 4: 1633-1637.

In some embodiments, said treating includes increasing the slope of eGFR computed from baseline to, e.g., week 26 and/or week 50 of a treatment period as compared to a control (e.g., untreated subjects or placebo-treated subjects).

In some embodiments, the subject was not diagnosed with rapid progressive glomerulonephritis as measured by an eGFR loss ≥30% over a period of 3 months prior to treatment with Compound 1 or the pharmaceutically acceptable salt thereof.

In some embodiments, the subject does not have a secondary etiology of IgAN (e.g., systemic lupus erythematosus (SLE), cirrhosis, or celiac disease).

In some embodiments, the subject does not have clinically active Henoch-Schonlein purpura (IgA vasculitis) requiring treatment.

In some embodiments, the subject has not received treatment with prednisone >about 20 mg/day or an equivalent thereof for >14 consecutive days or any other immunosuppressant (e.g., azathioprine or cyclophosphamide) prior to (e.g., within 6 months prior to) treatment with Compound 1 or the pharmaceutically acceptable salt thereof. Prednisone equivalents and dose relationships between prednisone and its equivalents are known in the art.

In some embodiments, the subject does not have blood pressure of 140/90 mmHg prior to treatment with Compound 1 or the pharmaceutically acceptable salt thereof confirmed on 2 measurements >30 minutes apart.

In some embodiments, the subject does not have a body mass index ≥38 kg/m² prior to treatment with Compound 1 or the pharmaceutically acceptable salt thereof.

In some embodiments, the subject is restricted from receiving treatment with hydroxychloroquine.

In some embodiments, the subject is restricted from receiving treatment with an immunosuppressive agent (e.g., MMF).

In some embodiments, the subject is restricted from receiving treatment with a systemic corticosteroid for >14 consecutive days.

### Pharmaceutical Compositions

The disclosure also relates to use of pharmaceutical compositions comprising Compound 1 and or a pharmaceutically acceptable salt thereof. Any suitable pharmaceutical compositions and formulations, as well as suitable methods for formulating and suitable routes and suitable sites of administration, are within the scope of this disclosure. Also, unless otherwise stated, any suitable dosage(s) and frequency of administration are contemplated.

Unless otherwise noted, the dosage level of Compound 1 or a pharmaceutically acceptable salt thereof can be any suitable level. In some embodiments, the dosage levels of Compound 1 or a pharmaceutically acceptable salt thereof for a subject can generally be between about 1 mg/kg and about 100 mg/kg (e.g., between about 2 mg/kg and about 50 mg/kg, between about 5 mg/kg and about 25 mg/kg), per treatment.

The compositions can be administered to a human subject using a variety of methods that depend, in part, on the route of administration. The route can be, e.g., oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

In some embodiments, a composition is formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which compositions can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

A suitable dose of Compound 1 or a pharmaceutically acceptable thereof which is capable of treating LN and/or IgAN in a subject, can depend on a variety of factors including, e.g., the age, gender, and weight of a subject to be treated and the particular inhibitor compound used. Other factors affecting the dose administered to the subject include, e.g., the severity of LN and/or IgAN. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse). A pharmaceutical composition can include a therapeutically effective amount of Compound 1 or a pharmaceutically acceptable salt thereof. Such effective amounts can be readily determined by one of ordinary skill in the art.

### Kits and Unit Dosage Forms

Also provided herein are kits that include Compound 1 or a pharmaceutically acceptable salt thereof in a therapeutically effective amount (e.g., in a pharmaceutical composition) for use in any one or more of the methods disclosed herein. The kit may optionally include instructions, e.g., comprising administration schedules, to allow a practitioner (e.g., a physician, nurse, or patient) to administer Compound 1 or the pharmaceutically acceptable salt thereof, e.g., in a pharmaceutical composition further including a pharmaceutically acceptable carrier) contained therein to a patient having LN and/or IgAN. The kit may further include a syringe.

Kits can optionally include multiple packages of the single-dose pharmaceutical compositions each containing an effective amount of Compound 1 or a pharmaceutically acceptable salt thereof e.g., in a pharmaceutical composition) for a single administration in accordance with the methods provided above. Instruments or devices for administering Compound 1 or a pharmaceutically acceptable salt thereof (e.g., in a pharmaceutical composition) may also be included in the kits. A kit may provide one or more pre-filled syringes containing an effective amount of Compound 1 or a pharmaceutically acceptable salt thereof, e.g., in a pharmaceutical composition).

The following examples are merely illustrative and should not be construed as limiting the scope of this disclosure in any way as many variations and equivalents will become apparent to those skilled in the art upon reading the present disclosure. The contents of all references, accessioned entries (e.g., PUBMED, GENBANK, UNIPROT, PUBCHEM entries), patents, and patent applications cited throughout this application are expressly incorporated herein by reference.

### EXAMPLES

### Example 1. A Phase 2, Randomized, Double-Blind, Placebo-Controlled, Dose-Finding Study to Evaluate the Efficacy and Safety of Compound 1 (ALXN2050; Vemircopan) in Adult Participants with Proliferative Lupus Nephritis (LN) on Immunoglobulin a Nephropathy (IgAN)

This is a Phase 2, randomized, double-blind, placebo-controlled, multicenter study of ALXN2050 in addition to background therapy consistent with the standard of care in adult participants (≥18 to ≤75 years of age) with either LN or IgAN.

Participants in the LN cohort must have a diagnosis of LN with an active flare based on kidney biopsy, estimated glomerular filtration rate (eGFR)>30 mL/min/1.73 m², and proteinuria (defined as UPCR≥1 g/g from one 24-hour urine collection).

Participants in the IgAN cohort must have a diagnosis of IgAN based on kidney biopsy, eGFR >30 mL/min/1.73 m², and proteinuria defined as mean protein ≥1 g/24 hours from 2 valid 24-hour urine collections. Participants in the IgAN cohort must have been treated with stable doses of the maximum tolerated renin-angiotensin system (RAS)-inhibiting medications and have controlled, stable blood pressure (<140/90 mmHg) for 3 months prior to Screening.

The study consists of an up to 6-week Screening Period, a 26-week blinded Initial Evaluation Period, a 24-week blinded Extended Treatment Period, and an OLE Period of up to 2 years. Upon completion of the OLE Period or if a participant decides to withdraw from the study, all participants will be followed for safety for 30 days after the last dose of study intervention. Thus, the total treatment duration is 154 weeks, and the total study duration is up to 164 weeks.

All participants will receive background therapy consistent with the standard of care for participants with LN and IgAN throughout the study. Participants in the LN cohort will receive rescue therapy in the event of a protocol-defined Renal Flare or Extrarenal systemic lupus erythematosus (SLE) Flare and after Week 26, for Suboptimal Response. Approved novel treatments for LN can be used per the Investigator's discretion. However, if rescue treatments are initiated, the Investigator should consult the list of disallowed medications (see below). If rescue treatment is a prohibited medication, study intervention should be discontinued at least 3 days prior to initiation of the rescue therapy.

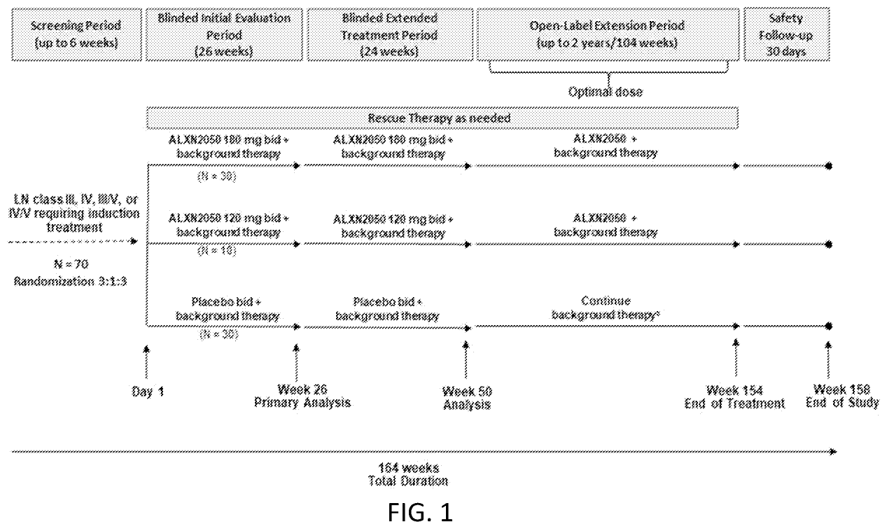

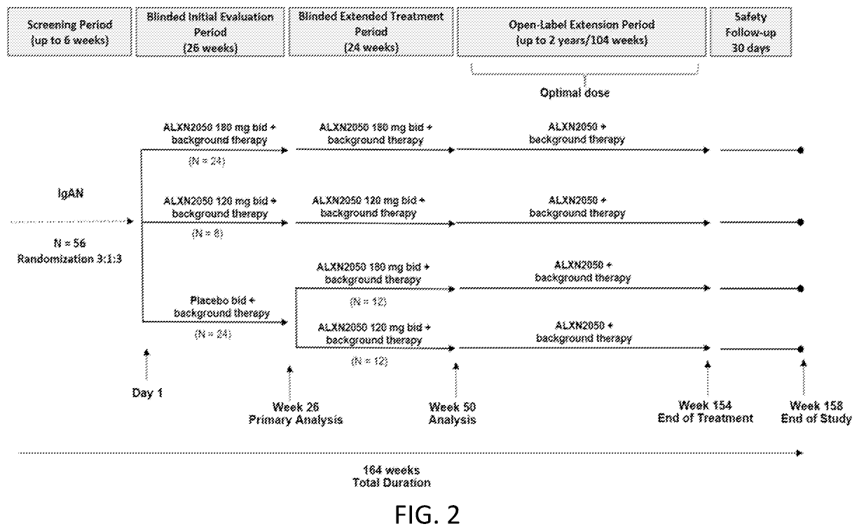

See study design schematics in FIG. 1 and FIG. 2 for LN and IgAN cohorts, respectively.

### Blinded Initial Evaluation Period

Approximately 126 adult participants with either LN or IgAN will be randomized into the study on Day 1 (approximately 70 participants in the LN cohort and approximately 56 participants in the IgAN cohort), as follows:
For each disease cohort, participants meeting eligibility criteria will be randomly assigned in a 3:1:3 ratio to receive ALXN2050 180 mg BID, ALXN2050 120 mg BID, or placebo BID (Table 7).
For the IgAN cohort, participants assigned to the placebo group will also be assigned to their study treatment for the blinded Extended Treatment Period (ALXN2050 180 mg BID or ALXN2050 120 mg BID) in a 1:1 allocation ratio at the time of randomization.

TABLE 7
Summary of Participants in Each Treatment Group
During the blinded Initial Evaluation Period

| | LN Cohort | IgAN Cohort |
|---|---|---|
| | Approximate Number | Approximate Number |
| Treatment Group | of Participants | of Participants |
| ALXN2050 180 mg BID | 30 | 24 |
| ALXN2050 120 mg BID | 10 | 8 |
| Placebo BID | 30 | 24 |

Abbreviations:
BID = twice daily;
IgAN = immunoglobulin A nephropathy;
LN = lupus nephritis

Stratification will be performed as follows:
For the LN cohort: by whether corticosteroid induction treatment was initiated prior to Screening versus during the Screening Period.
For the IgAN cohort: by mean proteinuria (1 to 2 g/day versus >2 g/day) from 2 valid 24-hour urine collections during the Screening Period.

After completion of the Day 1 Visit assessments, participants will receive the first dose of study intervention prior to leaving the clinic. Participants will continue to receive BID doses of study intervention throughout the blinded Initial Evaluation Period (see Table 17 below).

### Blinded Extended Treatment Period

After completion of the blinded Initial Evaluation Period (Week 26 Visit), participants will continue to receive study intervention during a blinded Extended Treatment Period for 24 weeks as follows:
Participants in the LN cohort will continue to receive their randomized allocation of study intervention (ALXN2050 180 mg, ALXN2050 120 mg, or placebo) BID. After Week 26, rescue therapy for participants with Suboptimal Response is allowed at the clinical discretion of the Investigator in conversation with the Medical Monitor.
Participants in the IgAN cohort randomized to active treatment will continue to receive their randomized allocation of study intervention (ALXN2050 180 mg or ALXN2050 120 mg) BID.
Participants in the IgAN cohort randomized to the placebo group will receive either ALXN2050 180 mg BID or ALXN2050 120 mg BID as assigned at the time of randomization.

### Open-Label Extension Treatment Period

After completion of all assessments at the end of the blinded Extended Treatment Period (Week 50 Visit), all participants will have the opportunity to enter an OLE Period and receive ALXN2050 for up to 2 years (104 weeks) if no relevant side effects (in the opinion of the Investigator) and no Treatment Failure (LN cohort) are present. During the OLE Period:
Participants in the LN cohort randomized to the active treatment groups will continue to receive the same dosing regimen as assigned during the blinded Extended Treatment Period.
Participants in the LN cohort randomized to the placebo group will no longer receive placebo and will continue to receive background therapy alone.
Participants in the IgAN cohort will continue to receive the same dosing regimen as assigned during the Blinded Extended Treatment Period.
Participants in both cohorts who were randomized to ALXN2050 and completed at least 50 weeks of treatment with ALXN2050 will receive the optimal dose level of ALXN2050 (180 mg or 120 mg BID) once an optimal dose is identified based on results of the primary analysis at Week 26 and/or Data Monitoring Committee (DMC) review of the safety and efficacy data.
Percentage change in proteinuria from baseline and change from baseline in eGFR at Week 102 and Week 154 will be assessed as exploratory efficacy endpoints. Safety and tolerability will also be evaluated continually throughout the OLE Period.

After completion of the study or after ED, a Safety Follow-up Visit will be conducted 30 days after the last dose of study intervention.

### Schedule of Activities

The schedule of activities (SoA) is provided as follows:
Screening Period and blinded Initial Evaluation Period: Screening to Week 26 (Day 183) Visit (LN cohort in Table 8 and IgAN cohort in Table 9)
Blinded Extended Treatment Period (LN cohort in Table 10 and IgAN cohort in Table 11)
Open-label Extension Period and Safety Follow-up Period (both LN and IgAN cohorts in Table 12)

TABLE 8
Schedule of Activities During Screening Period Blinded Initial Evaluation Period: Screening to Week 26 Visit (LN Cohort)

| Period | | | | | | | | | | |
|---|---|---|---|---|---|---|---|---|---|---|
| Screening | Initial Evaluation Period | | | | | | | | | |
| Visit | | | | | | | | | | |
| 1 | 2 | 3 | 4 | 5 | 6 | 7 | 8 | 9 | Evaluation | |
| Week | | | | | | | | | for Renal | |
| Up to 6 W | | W 2 | W 4 | W 8 | W 12 | W 16 | W 20 | W 26/ED^{b} | Flare and | Notes |
| Days and Window | | | | | | | | | Extrarenal | Unscheduled visits can be |
| | | D | D | D | D | D | D | D | SLE Flare | performed as needed. An ED |
| D −42 | | 15 ± | 29 ± | 57 ± | 85 ± | 113 ± | 141 ± | 183 ± | Assessment | Visit is required if participant |
| to −1 | D 1 | 3 | 3 | 5 | 5 | 5 | 5 | 5 | Visit^{a} | discontinues study early. |

General Assessments/Procedures

| | | | | | | | | | | |
|---|---|---|---|---|---|---|---|---|---|---|
| Informed consent | X | | | | | | | | | |
| Inclusion/exclusion | X | X | | | | | | | | Confirm eligibility prior to first |
| | | | | | | | | | | dose of study intervention at |
| | | | | | | | | | | Day 1; participants may be |
| | | | | | | | | | | rescreened once. |
| Demographics | X | | | | | | | | | |
| Medical history | X | | | | | | | | | |
| LN history/diagnosis | X | | | | | | | | | |
| Documentation of | X | | | | | | | | | Biopsy obtained ≤6 months |
| kidney biopsy | | | | | | | | | | prior to Screening or during |
| | | | | | | | | | | Screening. Send local |
| | | | | | | | | | | pathology report and |
| | | | | | | | | | | microscopy slides to Central |
| | | | | | | | | | | Pathology Laboratory. |
| Vaccination or | X | | | | | | | | | Prophylactic antibiotics for at |
| confirmation of | | | | | | | | | | least 2 weeks post vaccination |
| vaccination against | | | | | | | | | | if <2 weeks prior to first dose. |
| Neisseria meningitidis^{c} | | | | | | | | | | |
| Prior LN therapy | X | | | | | | | | | Record corticosteroid and |
| | | | | | | | | | | MMF usage. |
| Weight | X | X | | | | | | | X | |
| Height | X | | | | | | | | | |
| Pregnancy test | X | X | X | X | X | X | X | X | X | Serum tests required at |
| (WOCBP only) | | | | | | | | | | Screening and ED; urine tests |
| | | | | | | | | | | at all other visits. |
| FSH test^{d} | X | | | | | | | | | Only for females who are not |
| | | | | | | | | | | WOCBP. |
| HIV, HCV, and HBV | X | | | | | | | | | |
| Dispense participant | X | | | | | | | | | Instruct participants to carry |
| safety card | | | | | | | | | | safety card at all times and |
| | | | | | | | | | | bring it to scheduled visits. |

Efficacy Assessments

| 24-hour urine collection^{e} | X | | | | | | | | X | X | One collection is needed as |
|---|---|---|---|---|---|---|---|---|---|---|---|
| | | | | | | | | | | | soon as possible during |
| | | | | | | | | | | | Screening. Two separate |
| | | | | | | | | | | | collections must be obtained |
| | | | | | | | | | | | within 2 weeks prior to the |
| | | | | | | | | | | | Week 26 Visit. |
| Morning spot urine | X | X | X | X | X | X | X | X | X | X | Two consecutive spot urine |
| sample | | | | | | | | | | | samples at Week 16. |
| eGFR (measured by | X | X | X | X | X | X | X | X | X | X | One additional blood draw for |
| serum creatine) | | | | | | | | | | | eGFR (serum creatinine) is |
| | | | | | | | | | | | required within 2 weeks prior to |
| | | | | | | | | | | | the Week 26 visit |
| Predose blood sample | X | X | X | | X | | X | | X | | |
| for serum clinical | | | | | | | | | | | |
| complement tests | | | | | | | | | | | |
| Monitor for Renal Flare | | Continuous monitoring | | | | | | | | X | Document use of rescue |
| and/or Extrarenal SLE | | | | | | | | | | | therapy and/or repeat biopsy, if |
| Flare^{a} | | | | | | | | | | | applicable. |

Safety Assessments

| Physical examination, | X | X | | | | | | | X | | |
|---|---|---|---|---|---|---|---|---|---|---|---|
| including neurological | | | | | | | | | | | |
| assessments^{f} | | | | | | | | | | | |
| Abbreviated PE | | | X | X | X | X | X | X | | | |
| Vital signs | X | X | X | X | X | X | X | X | X | X | Vital signs to be taken predose |
| | | | | | | | | | | | at clinic visits. |
| ECG for LN patients on | X | X | X | X | X | X | X | X | X | X | |
| hydroxychloroquine | | | | | | | | | | | |
| ECG for LN patients not | X | X | | | | | | | X | | |
| on hydroxychloroquine | | | | | | | | | | | |
| Prior medications and | X | | | | | | | | | | |
| procedures | | | | | | | | | | | |
| Concomitant | | Continuous monitoring | | | | | | | | X | |
| medications and NPTP | | | | | | | | | | | |
| Background LN therapy | | Continuous monitoring | | | | | | | | X | |
| Adverse events | Continuous monitoring | | | | | | | | | X | |
| Predose blood sample | X | X | X | X | X | X | X | X | X | X | |
| for clinical chemistry, | | | | | | | | | | | |
| hematology, and | | | | | | | | | | | |
| coagulation | | | | | | | | | | | |
| Urinalysis and urine | X | X | X | X | X | X | X | X | X | X | |
| sediment | | | | | | | | | | | |
| Participant safety card | | X | X | X | X | X | X | X | X | X | Confirm participants carry |
| review | | | | | | | | | | | safety card at all times. |

PK and PD Assessments^{g}

| | | | | | | | | | |
|---|---|---|---|---|---|---|---|---|---|
| Predose blood sample | X | X | X | X | X | X | X | X | See footnote for sampling |
| for PK | | | | | | | | | timepoints. |
| Predose blood sample | X | X | X | X | X | X | X | X | |
| for Bb and AP activity | | | | | | | | | |
| Postdose blood sample | X | X | | | | | | | |
| for PK | | | | | | | | | |
| Postdose blood sample | X | X | | | | | | | |
| for Bb and AP activity | | | | | | | | | |

Exploratory Assessments

| | | | | | | | | |
|---|---|---|---|---|---|---|---|---|
| EQ-5D-5L | | X | | X | X | X | | If possible, complete the PROS |
| SF-36 | | X | | X | X | X | | prior to procedures and |
| FACIT-Fatigue | | X | | X | X | X | | intervention administration. |
| SLEDAI-2K | | X | | | | X | X | Required at Day 1 and |
| | | | | | | | | Week 26/ED; perform as |
| | | | | | | | | needed for evaluation of |
| | | | | | | | | Extrarenal SLE Flare. |
| Blood and urine | X | X | X | X | X | X | X | Collect predose on in-clinic |
| samples for biomarkers | | | | | | | | dosing days. |
| Blood and urine | X | X | X | X | X | X | | To be performed at selected |
| samples for RTCA | | | | | | | | sites only |
| Blood sample for | X | X | | | | X | X | |
| autoantibodies | | | | | | | | |
| Optional kidney biopsy^{h} | | | | | | | X | Send local pathology report |
| | | | | | | | | and microscopy slides to the |
| | | | | | | | | Central Pathology Laboratory. |

Study Intervention

| | | | | | | | | | |
|---|---|---|---|---|---|---|---|---|---|
| Randomization | X | | | | | | | | Review eligibility prior to |
| | | | | | | | | | randomization. |
| Morning dose | X | X | X | X | X | X | X | Xⁱ | Administer after all other |
| administered in clinic | | | | | | | | | required tests/procedures. |
| Study intervention | X | X | X | X | X | X | X | X | Participants on study |
| compliance and | | | | | | | | | intervention should bring all |
| accountability | | | | | | | | | unused study intervention at |
| | | | | | | | | | each clinic visit for tablet |
| | | | | | | | | | counting |
| Study intervention | X | X | X | X | X | X | X | X | Participants on study |
| dispensing | | | | | | | | | intervention will be provided |
| | | | | | | | | | with sufficient study |
| | | | | | | | | | intervention to last until their |
| | | | | | | | | | next study visit (except at ED). |

^{a}Renal Flare (as defined herein in) and/or Severe Extrarenal SLE Flare (as defined herein) may occur at any time during the study. Evaluation of Renal Flare requires a UPCR from a spot urine sample that is confirmed on a 24-hour urine collection, as well as 2 serum creatinine samples obtained within a 2-week period. Evaluation of Renal and Extrarenal SLE Flares must be performed as soon as possible upon notification to the Investigator of symptom onset. If Renal Flare or Extrarenal SLE Flare occurs between scheduled visits, only the assessments for the Renal Flare/Extrarenal SLE Flare Visit are needed. If Renal Flare or Extrarenal SLE Flare occurs on a scheduled visit, all scheduled assessments should be performed for that visit as well as any additional assessments required for the evaluation of the flare.
^{b}For participants who discontinue study intervention prior to Week 50, every effort should be made to have the participant continue the study visits as per the SoA through the Week 50. A Safety Follow-up Visit should be performed 30 days after the last dose of study intervention. If the participant does not agree to continue with the study visits after study intervention is discontinued, the ED Visit should be performed as soon as possible, and a Safety Follow-up Visit should be performed 30 days after the last dose of study intervention (refer to Table 12 for the Safety Follow-up Visit procedures).
^{sc}To reduce the risk of meningococcal infection (Neisseria meningitidis), all participants must be vaccinated against meningococcal infection within 3 years or before the administration of study intervention on Day 1. Participants who initiate study intervention less than 2 weeks after receiving a meningococcal vaccine must receive treatment with appropriate prophylactic antibiotics until at least 2 weeks after vaccination.
^{d}FSH test need not be done if documentation confirming postmenopausal status is available.
^{e}The 24-hour urine collection must be obtained prior to or >7 days after biopsy procedures. Renal Flare requires a single 24-hour urine collection for confirmation.
^{f}Perform a symptom based neurologic examination if participant has complaints or clinical findings attributable to the CNS and if positive for findings, full neurologic examination to be performed at each assessment timepoint.
^{g}Predose: collect within 0.5 h predose on indicated in-clinic dosing days. Postdose: approximately 50% participant in each treatment arm will be assigned to PK/PD sample collection at 2 ± 0.5 h, 4 ± 0.5 h, and 6 ± 0.5 h postdose on Day 1 and at 2 ± 0.5 h, 4 ± 0.5 h, 6 ± 0.5 h postdose at Week 2. For the other approximate 50% of participant, samples will be collected at 2 ± 0.5 h postdose on Day 1 and 2 ± 0.5 h postdose at Week 2
^{h}Participants may receive a kidney biopsy for clinical reasons or for evaluation of a Renal Flare (as defined herein) at the discretion of the Investigator. The local pathology report and microsection slides from kidney biopsies performed at other times during the study prior to Week 26 should also be sent to the Central Pathology Laboratory for review as soon as possible.
ⁱThe primary efficacy endpoint assessment will be obtained prior to dosing on Day 183. Dosing on Day 183 is the start of the blinded Extended Treatment Period.
Note:
If possible, all assessments should be performed prior to administration of study intervention on in-clinic dosing days, unless otherwise specified.
Abbreviations:
AP = alternative pathway;
Bb = Bb fragment of complement factor B;
CNS = central nervous system;
D = day;
ECG = electrocardiogram;
ED = early discontinuation;
EQ-5D-5L = European Quality of Life Health 5-item questionnaire dimensions 5 level;
FACIT = Functional Assessment of Chronic Illness Therapy;
FSH = follicle stimulating hormone;
HBV = hepatitis B virus;
HCV = hepatitis C virus;
HIV = human immunodeficiency virus;
LN = lupus nephritis;
MMF = mycophenolate mofetil;
NPTP = nonpharmacologic therapies and procedures;
PD = pharmacodynamics;
PE = physical examination;
PK = pharmacokinetics;
PRO = participant-reported outcome;
RTCA = real time complement activity;
SLE = systemic lupus erythematosus;
SF-36 = Short Form (36) Health Survey;
SLEDAI-2K = Systemic Lupus Erythematosus Disease Activity Index 2000 Safety of Estrogens in Lupus Erythematosus National Assessment (SELENA) Modification;
SoA = schedule of assessments;
UPCR = urine protein to creatinine ratio;
W = week;
WOCBP = women of childbearing potential

TABLE 9
Schedule of Activities During Screening and the Blinded Initial Evaluation Period: Screening to Week 26 Visit (IgAN Cohort)

| Period | | | | | | | |
|---|---|---|---|---|---|---|---|
| Screening | Initial Evaluation Period | | | | | | |
| Visit | | | | | | | |
| 1 | 2 | 3 | 4 | 5 | 6 | 7 | |
| Week | | | | | | | Notes |
| Up to 6 W | | W 2 | W 6 | W 12 | W 18 | W 26/ED^{a} | Unscheduled visits can be |
| Days and Window | | | | | | | performed as needed. |
| | | D | D | D | D | D | An ED Visit is required |
| D −42 | | 15 ± | 43 ± | 85 ± | 127 ± | 183 ± | if participant discontinues |
| to −1 | D 1 | 3 | 5 | 5 | 5 | 5 | study early. |

General Assessments/Procedures

| | | | | | | | | |
|---|---|---|---|---|---|---|---|---|
| Informed consent | X | | | | | | | |
| Inclusion/exclusion | X | X | | | | | | Confirm eligibility prior to first |
| | | | | | | | | dose of study intervention at Day 1; |
| | | | | | | | | participants may be rescreened once. |
| Demographics | X | | | | | | | |
| Medical history | X | | | | | | | |
| IgAN history/diagnosis | X | | | | | | | |
| Documentation of kidney | X | | | | | | | Kidney biopsy performed during or prior |
| biopsy | | | | | | | | to Screening (any time prior to Day 1). |
| | | | | | | | | Send local pathology report and microscopy |
| | | | | | | | | slides to Central Pathology. |
| Prior IgAN therapy | X | | | | | | | Record ACEi/ARB usage. |
| Vaccination or confirmation | X | | | | | | | Prophylactic antibiotics for at least 2 |
| of vaccination against | | | | | | | | weeks post vaccination if <2 weeks prior |
| N meningitidis ^{b} | | | | | | | | to first dose. |
| Weight | X | X | | | | | X | |
| Height | X | | | | | | | |
| HIV, HCV, and HBV | X | | | | | | | |
| Pregnancy test (WOCBP | X | X | X | X | X | X | X | Serum tests required at Screening and ED; |
| only) | | | | | | | | urine tests at all other visits. |
| FSH test^{c} | X | | | | | | | Only for females who are not WOCBP. |
| Dispense participant safety | X | | | | | | | Instruct participants to carry safety card |
| card | | | | | | | | at all times and bring to scheduled visits. |

Efficacy Assessments

| | | | | | | | | |
|---|---|---|---|---|---|---|---|---|
| 24-hour urine collection^{d} | X | | | | | | X | Two separate, complete, and valid 24- |
| | | | | | | | | hour urine collections are required during |
| | | | | | | | | Screening and within 2 weeks prior to the |
| | | | | | | | | Week 26 Visit. |
| Morning spot urine sample | X | X | X | X | X | X | X | Two consecutive spot urine samples at |
| | | | | | | | | Week 18. |
| eGFR (measured by serum | X | X | X | X | X | X | X | |
| creatine) | | | | | | | | |
| Predose blood sample for | X | X | X | | X | X | X | |
| serum clinical complement | | | | | | | | |
| tests | | | | | | | | |

Safety Assessments

| Physical examination, | X | X | | | | | X | |
|---|---|---|---|---|---|---|---|---|
| including neurological | | | | | | | | |
| assessments^{e} | | | | | | | | |
| Abbreviated PE | | | X | X | X | X | | |
| Vital signs | X | X | X | X | X | X | X | Vital signs to be taken prior to dosing |
| | | | | | | | | at clinic visits. |
| ECG | X | X | | | | | X | |
| Prior medications and | X | | | | | | | |
| procedures | | | | | | | | |
| Concomitant medications | | Continuous monitoring | | | | | | |
| and NPTP | | | | | | | | |
| Background IgAN therapy | | Continuous monitoring | | | | | | |
| Adverse events | Continuous monitoring | | | | | | | |
| Predose blood sample for | X | X | X | X | X | X | X | |
| clinical chemistry, | | | | | | | | |
| hematology, and | | | | | | | | |
| coagulation | | | | | | | | |
| Urinalysis and urine | X^{f} | X | X | X | X | X | X | |
| sediment | | | | | | | | |
| Participant safety card | | X | X | X | X | X | X | Confirm participants carry safety |
| review | | | | | | | | card at all times. |

PK and PD Assessments^{g}

| | | | | | | | |
|---|---|---|---|---|---|---|---|
| Predose blood sample for | X | X | X | X | X | X | See footnote for sampling timepoints. |
| PK | | | | | | | |
| Predose blood sample for | X | X | X | X | X | X | |
| Bb and AP activity | | | | | | | |
| Postdose blood sample for | X | X | | | | | |
| PK | | | | | | | |
| Postdose blood sample for | X | X | | | | | |
| Bb and AP activity | | | | | | | |

Exploratory Assessments

| | | | | | | | | |
|---|---|---|---|---|---|---|---|---|
| EQ-5D-5L | | X | | X | X | | X | If possible, complete the PROs prior |
| SF-36 | | X | | X | X | | X | to procedures and intervention |
| | | | | | | | | administration. |
| Blood and urine samples | X | X | X | | X | X | X | Collect predose on in-clinic dosing |
| for biomarkers | | | | | | | | days. |
| Blood and urine samples | X | X | X | | X | X | X | To be performed at selected sites only |
| for RTCA | | | | | | | | |

Study Intervention

| | | | | | | | |
|---|---|---|---|---|---|---|---|
| Randomization | X | | | | | | Review eligibility prior to randomization. |
| Morning dose administered | X | X | X | X | X | X^{h} | Administer dose after all other required |
| in clinic | | | | | | | tests/procedures. |
| Study intervention | X | X | X | X | X | X | Participants on study intervention should |
| compliance and | | | | | | | bring all unused study intervention at each |
| accountability | | | | | | | clinic visit for tablet counting. |
| Study intervention | X | X | X | X | X | X | Participants on study intervention will |
| dispensing | | | | | | | be provided with sufficient study |
| | | | | | | | intervention to last until their next |
| | | | | | | | study visit (except at ED). |

^{a}For participants who discontinue study intervention prior to Week 50, every effort should be made to have the participant continue the study visits as per the SoA through the Week 50. A Safety Follow-up Visit should be performed 30 days after the last dose of study intervention. If the participant does not agree to continue with the study visits after study intervention is discontinued, the ED Visit should be performed as soon as possible, and a Safety Follow-up Visit should be performed 30 days after the last dose of study intervention. Refer to Table 12 for the Safety Follow-up Visit procedures.
^{S b} To reduce the risk of meningococcal infection (N meningitidis), all participants must be vaccinated against meningococcal infection within 3 years or before the administration of study intervention on Day 1. Participants who initiate study intervention less than 2 weeks after receiving a meningococcal vaccine must receive treatment with appropriate prophylactic antibiotics until at least 2 weeks after vaccination.
^{c}FSH test need not be done if documentation confirming postmenopausal status is available.
^{d}The 24-hour urine collection must be obtained prior to or >7 days after biopsy procedures.
^{e}Perform a symptom based neurologic examination if participant has complaints or clinical findings attributable to the CNS and if positive for findings, full neurologic examination to be performed at each assessment timepoint.
^{f}For participants in the IgAN cohort, eligibility for hematuria can be determined via the local laboratory.
^{g}Predose: collect within 0.5 h predose on indicated in-clinic dosing days. Postdose: approximately 50% participant in each treatment arm will be assigned to PK/PD sample collection at 2 ± 0.5 h, 4 ± 0.5 h, 6 ± 0.5 h postdose on Day 1 and at 2 ± 0.5 h, 4 ± 0.5 h, 6 ± 0.5 h postdose at Week 2. For the other approximately 50% of participant, samples will be collected at 2 ± 0.5 h postdose on Day 1 and 2 ± 0.5 h postdose at Week 2.
^{h}The primary efficacy endpoint assessment will be obtained prior to dosing on Day 183. Dosing on Day 183 is the start of the blinded Extended Treatment Period.
Note:
If possible, all assessments should be performed prior to administration of study intervention on in-clinic dosing days, unless otherwise specified.
Abbreviations: ACE = angiotensin-converting enzyme; AP = alternative pathway; ARB = angiotensin II receptor blocker; Bb = Bb fragment of complement factor B; CNS = central nervous system; D = day; ECG = electrocardiogram; ED = early discontinuation; EQ-5D-5L = European Quality of Life Health 5-item questionnaire dimensions 5 level; FSH = follicle-stimulating hormone; HBV = hepatitis B virus; HCV = hepatitis C virus; HIV = human immunodeficiency virus; IgAN = immunoglobulin A nephropathy; NPTP = nonpharmacologic therapies and procedures; PD = pharmacodynamics; PE = physical examination; PK = pharmacokinetics; PRO = participant-reported outcome; RTCA = real time complement activity; SF-36 = Short Form (36) Health Survey; SoA = schedule of assessments; W = week; WOCBP = women of childbearing potential.

TABLE 10
Schedule of Activities During the Blinded Extended Treatment Period: Week 34 to Week 50 Visits (LN Cohort)

| Period | | | | |
|---|---|---|---|---|
| Extended Treatment Period | | | | |
| Visit | | | | |
| 10 | 11 | 12 | Evaluation for Renal | |
| Week | | | Flare and Extrarenal | Notes |
| W 34 | W 42 | W 50/ED^{b} | SLE Flare, and | Unscheduled visits can be performed as needed. An |
| Days and Window | | | Suboptimal Response | ED Visit is required if participant discontinues study |
| D 239 ± 7 | D 295 ± 7 | D 351 ± 7 | Assessment Visit^{a} | early. |

General Assessments/Procedures

| | | | | |
|---|---|---|---|---|
| Weight | | | X | |
| Pregnancy test (WOCBP only) | X | X | X | Serum pregnancy test required at ED; urine pregnancy |
| | | | | tests at all other visits. |

Efficacy Assessments

| 24-hour urine collection | | | X | X | Two separate 24-hour urine collections required within |
|---|---|---|---|---|---|
| | | | | | 2 weeks prior to the Week 50 Visit. Renal Flare requires |
| | | | | | a single 24-hour urine collection for confirmation |
| Morning spot urine sample | X | X | X | X | |
| eGFR (measured by serum | X | X | X | X | One additional blood draw for eGFR (serum creatinine) |
| creatine) | | | | | is required within 2 weeks prior to the Week 50 visit |
| Predose blood sample for serum | X | X | X | X | |
| clinical complement tests | | | | | |
| Monitor for Renal Flare and/or | Continuous monitoring | | | X | Document use of rescue therapy and/or repeat biopsy, if |
| Extrarenal SLE Flare and/or | | | | | applicable. |
| Suboptimal Response^{a} | | | | | |

Safety Assessments

| Physical examination, including | | | X | | |
|---|---|---|---|---|---|
| neurological assessments^{c} | | | | | |
| Abbreviated PE | X | X | | | |
| Vital signs | X | X | X | X | Vital signs to be taken predose at clinic visits. |
| ECG for LN patients on | X | X | X | X | |
| hydroxychloroquine | | | | | |
| ECG for LN patients not on | | | X | | |
| hydroxychloroquine | | | | | |
| Concomitant medications and | Continuous monitoring | | | X | |
| NPTP | | | | | |
| Background LN therapy | Continuous monitoring | | | X | See details on corticosteroid taper herein. |
| Adverse events | Continuous monitoring | | | X | |
| Predose blood sample for | X | X | X | X | |
| clinical chemistry, | | | | | |
| hematology, and coagulation | | | | | |
| Urinalysis and urine sediment | X | X | X | X | |
| Participant safety card review | X | X | X | X | Confirm participants carry safety card at all times. |

PD and PK Assessments

| | | | | |
|---|---|---|---|---|
| Predose blood samples for PK | X | X | X | X |
| Predose blood sample for | X | X | X | X |
| Bb and AP activity | | | | |

Exploratory Assessments

| | | | | | |
|---|---|---|---|---|---|
| SF-36 | X | X | X | | PROs to be performed as early as possible during visits. |
| EQ-5D-5L | X | X | X | | |
| FACIT-Fatigue | X | X | X | | |
| SLEDAI-2K | | | X | X | Required at Week 50/ED, perform as needed for |
| | | | | | evaluation of Extrarenal SLE Flare. |
| Blood and urine samples for | X | X | X | X | Collect predose on in-clinic dosing days. |
| biomarkers | | | | | |
| Blood sample for autoantibodies | | | X | X | |
| Optional kidney biopsy^{d} | | | X | X | Send local pathology report and microscopy slides to the |
| | | | | | Central Pathology Laboratory. |

Study Intervention^{e}

| | | | | |
|---|---|---|---|---|
| Morning dose administered | X | X | X | Administer after all other required tests/procedures. |
| in clinic | | | | |
| Study intervention compliance | X | X | X | Participants on study intervention should bring all unused |
| and accountability | | | | study intervention at each clinic visit for tablet counting. |
| Study intervention dispensing | X | X | X | Participants on study intervention will be provided with |
| | | | | sufficient study intervention to last until their next study |
| | | | | visit (except at ED). |

^{a}Renal Flare (as defined herein) and/or Severe Extrarenal SLE Flare (as defined herein) may occur at any time during the study. Suboptimal Response may occur after Week 26. Evaluation of Renal Flare requires a UPCR from a spot urine sample that is confirmed on a 24-hour urine collection, as well as 2 serum creatinine samples obtained within a 2-week period. Evaluation of Renal and Extrarenal SLE Flares must be performed as soon as possible upon notification to the Investigator of symptom onset. If Renal Flare or Extrarenal SLE Flare occur between scheduled visits, only the assessments for the Renal Flare/Extrarenal SLE Flare Visit are needed. If Renal Flare or Extrarenal SLE Flare occur on a scheduled visit, all scheduled assessments should be performed for that visit as well as any additional assessments required for the evaluation of the flare.
^{b}For participants who discontinue study intervention prior to Week 50, every effort should be made to have the participant continue the study visits as per the SoA through the Week 50. A Safety Follow-up Visit should be performed 30 days after the last dose of study intervention. If the participant does not agree to continue with the study visits after study intervention is discontinued, the ED Visit should be performed as soon as possible, and a Safety Follow-up Visit should be performed 30 days after the last dose of study intervention. Refer to Table 12 for the Safety Follow-up Visit procedures.
^{c}Perform a symptom based neurologic examination if participant has complaints or clinical findings attributable to the CNS and if positive for findings, full neurologic examination to be performed at each assessment timepoint.
^{d}Participants will be asked to undergo an optional repeat kidney biopsy after completion of the blinded Extended Treatment Period. If a participant agrees to a repeat renal biopsy, it should be performed at the Week 50 Visit or within 4 weeks (by Week 54).
^{e}During the blinded Extended Treatment Period, participants in the LN cohort will continue to receive their randomized allocation of study intervention.
Note:
If possible, all assessments should be performed prior to administration of study intervention on in-clinic dosing days, unless otherwise specified.
Abbreviations:
AP = alternative pathway;
Bb = Bb fragment of complement factor B;
BP = blood pressure;
CNS = central nervous system;
D = day;
ECG = electrocardiogram;
ED = early discontinuation;
EQ-5D-5L = European Quality of Life Health 5-item questionnaire dimensions 5 level;
FACIT = Functional Assessment of Chronic Illness Therapy;
LN = lupus nephritis;
NPTP = nonpharmacologic therapies and procedures;
PD = pharmacodynamics;
PE = physical examination;
PK = pharmacokinetics;
PRO = participant-reported outcome;
SF-36 = Short Form (36) Health Survey;
SLE = systemic lupus erythematosus;
SLEDAI-2K = Systemic Lupus Erythematosus Disease Activity Index 2000 Safety of Estrogens in Lupus Erythematosus National Assessment (SELENA) Modification;
SoA = schedule of assessments;
UPCR = urine protein to creatinine ratio;
W = week;
WOCBP = women of childbearing potential

TABLE 11
Schedule of Activities During the Blinded Extended Treatment Period: Week 28 to Week 50 Visits (IgAN Cohort)

| Period | | | | | |
|---|---|---|---|---|---|
| Extended Treatment Period | | | | | |
| Visit | | | | | |
| 8 | 9 | 10 | 11 | 12 | |
| Week | | | | | |
| | | | | W 50/ | |
| W 28 | W 34 | W 38 | W 42 | ED^{a} | Notes |
| Days and Window | | | | | Unscheduled visits can be performed |
| D 197 ± | D 239 ± | D 267 ± | D 295 ± | D 351 ± | as needed. An ED Visit is required if |
| 7 | 7 | 7 | 7 | 7 | participant discontinues study early. |

General Assessments/Procedures

| | | | | | | |
|---|---|---|---|---|---|---|
| Weight | | | | | X | |
| Pregnancy test (WOCBP only) | X | X | X | X | X | Serum pregnancy test required at ED; |
| | | | | | | urine pregnancy tests at all other visits. |

Efficacy Assessments

| | | | | | | |
|---|---|---|---|---|---|---|
| 24-hour urine collection | | | | | X | Two separate 24-h urine collections |
| | | | | | | required within 2 weeks prior to the |
| | | | | | | Week 50 Visit. |
| Morning spot urine sample | X | X | X | X | X | |
| eGFR (measured by serum creatine) | X | X | X | X | X | |
| Predose blood sample for serum clinical | X | X | X | X | X | |
| complement tests | | | | | | |

Safety Assessments

| Physical examination, including neurological | | | | | X | |
|---|---|---|---|---|---|---|
| assessments^{b} | | | | | | |
| Abbreviated PE | X | X | X | X | | |
| Vital signs | X | X | X | X | X | Vital signs to be taken predose at clinic |
| | | | | | | visits. |
| ECG | | | | | X | |
| Concomitant medications and NPTP | Continuous monitoring | | | | | |
| Background IgAN therapy | Continuous monitoring | | | | | |
| Adverse events | Continuous monitoring | | | | | |
| Predose blood sample for clinical chemistry, | X | X | X | X | X | |
| hematology, and coagulation | | | | | | |
| Urinalysis and urine sediment | X | X | X | X | X | |
| Participant safety card review | X | X | X | X | X | Confirm participants carry safety card at |
| | | | | | | all times. |

PK and PD Assessments

| | | | | | |
|---|---|---|---|---|---|
| Predose blood samples for PK | X | X | X | X | X |
| Predose blood sample for Bb and AP activity | X | X | X | X | X |

Exploratory Assessments

| | | | | | | |
|---|---|---|---|---|---|---|
| SF-36 | | | X | | X | PROs to be performed as early as |
| EQ-5D-5L | | | X | | X | possible during visits. |
| Blood and urine samples for biomarkers | X | X | X | X | X | Collect predose on in-clinic dosing days. |

Study Intervention^{c}

| | | | | | | |
|---|---|---|---|---|---|---|
| Morning dose administered in clinic | X | X | X | X | X | Administer dose after all other required |
| | | | | | | tests/procedures. |
| Study intervention compliance and accountability | X | X | X | X | X | Participants on study intervention should |
| | | | | | | bring all unused study intervention at each |
| | | | | | | clinic visit for tablet counting. |
| Study intervention dispensing | X | X | X | X | X | Participants on study intervention will be |
| | | | | | | provided with sufficient study intervention |
| | | | | | | to last until their next study visit (except at |
| | | | | | | ED). |

^{a}For participants who discontinue study intervention prior to Week 50, every effort should be made to have the participant continue the study visits as per the SoA through the Week 50. A Safety Follow-up Visit should be performed 30 days after the last dose of study intervention. If the participant does not agree to continue with the study visits after study intervention is discontinued, the ED Visit should be performed as soon as possible, and a Safety Follow-up Visit should be performed 30 days after the last dose of study intervention. Refer to Table 12 for the Safety Follow-up Visit procedures.
^{b}Perform a symptom based neurologic examination if participant has complaints or clinical findings attributable to the CNS and if positive for findings, full neurologic examination to be performed at each assessment timepoint.
^{c}During the blinded Extended Treatment Period, participants in the IgAN placebo group will switch to receive either ALXN2050 180 mg BID or ALXN2050 120 mg BID. Participants in the IgAN cohort randomized to active treatment will continue to receive their randomized allocation of study intervention.
Note:
If possible, all assessments should be performed prior to administration of study intervention on in-clinic dosing days, unless otherwise specified.
Abbreviations:
AP = alternative pathway;
Bb = Bb fragment of complement factor B;
bid = twice daily;
CNS = central nervous system;
D = day;
ECG = electrocardiogram;
ED = early discontinuation;
EQ-5D-5L = European Quality of Life Health 5-item questionnaire dimensions 5 level;
IgAN = immunoglobulin A nephropathy;
NPTP = nonpharmacologic therapies and procedures;
PD = pharmacodynamics;
PE = physical examination;
PK = pharmacokinetics;
PRO = participant-reported outcome;
SF-36 = Short Form (36) Health Survey;
SoA = schedule of assessments;
W = week;
WOCBP = women of childbearing potential

TABLE 12
Schedule of Activities During the Open-label Extension Period and Safety Follow-up Period (Both LN and IgAN)

| | | | | | | | | | | Evaluation | Safety | |
|---|---|---|---|---|---|---|---|---|---|---|---|---|
| | | | | | | | | | | for Renal | Follow- | |
| | Period | | | | | | | | | Flare, | up | |
| | Adhocvisit^{a} | Open-label Extension Period | | | | | | | | Extrarenal | Period | Notes |
| | Week | | | | | | | | | SLE Flare, | 4 weeks | Unscheduled visits |
| | After | W | W | W | W | W | W | W | W 154/ | and | post last | can be performed |
| | W 50 | 63 | 76 | 89 | 102 | 115 | 128 | 141 | ED^{b} | Suboptimal | dose | as needed. A ED |
| | Days and Window | | | | | | | | | Response | 30 (±3) | Visit required |
| | | D | D | D | D | D | D | D | D | Assessment | days | if participant |
| | | 442 ± | 533 ± | 624 ± | 715 ± | 806 ± | 897 ± | 988 ± | 1079 ± | Visit | post last | discontinues |
| | | 14 | 14 | 14 | 14 | 14 | 14 | 14 | 14 | (LN only)^{c} | dose | study early |
| General Assessments/ | | | | | | | | | | | | |
| Procedures | | | | | | | | | | | | |
| Weight | | | | | | | | | X | | | |
| Pregnancy test (WOCBP | | X | X | X | X | X | X | X | X | | X | Serum test required |
| only) | | | | | | | | | | | | at ED; urine tests |
| | | | | | | | | | | | | at all other visits. |
| Efficacy Assessments | | | | | | | | | | | | |
| 24-hour urine collection | | | | | X | | | | X | X | | Obtain one 24-hour |
| | | | | | | | | | | | | urine collection. |
| Morning spot urine sample | | X | X | X | X | X | X | X | X | X | | |
| Monitor for Renal Flare | Continuous monitoring | | | | | | | | | X | | Document use of |
| Extrarenal SLE Flare, | | | | | | | | | | | | rescue therapy and/ |
| and/or Suboptimal | | | | | | | | | | | | or repeat biopsy, |
| Response (LN only) | | | | | | | | | | | | if applicable. |
| Blood samples for | | | | | | | | | | X | | |
| autoantibodies (LN only) | | | | | | | | | | | | |
| Safety Assessments | | | | | | | | | | | | |
| Physical examination, | | | | | | | | | X | | | |
| including neurological | | | | | | | | | | | | |
| assessments^{d} | | | | | | | | | | | | |
| Abbreviated PE | | X | X | X | X | X | X | X | | | X | |
| ECG (only for LN patients | X | X | X | X | X | X | X | X | X | X | X | |
| receiving | | | | | | | | | | | | |
| hydroxychloroquine) | | | | | | | | | | | | |
| Vital signs | X | X | X | X | X | X | X | X | X | X | X | Vital signs to be |
| | | | | | | | | | | | | taken prior to dosing |
| | | | | | | | | | | | | at clinic visits. |
| Concomitant medications | | Continuous monitoring | | | | | | | | X | X | |
| and NPTP | | | | | | | | | | | | |
| Background LN or IgAN | | Continuous monitoring | | | | | | | | X | X | |
| therapy | | | | | | | | | | | | |
| Adverse events | | Continuous monitoring | | | | | | | | X | X | |
| Blood sample for clinical | | X | X | X | X | X | X | X | X | X | | |
| chemistry, hematology, and | | | | | | | | | | | | |
| coagulation | | | | | | | | | | | | |
| Urinalysis and urine | | X | X | X | X | X | X | X | X | X | | |
| sediment | | | | | | | | | | | | |
| Participant safety card | | X | X | X | X | X | X | X | X | X | | Confirm participants |
| review | | | | | | | | | | | | carry safety card |
| | | | | | | | | | | | | at all times. |
| Study Intervention^{e} | | | | | | | | | | | | |
| Study intervention | | X | X | X | X | X | X | X | X | | | Participants on study |
| compliance and | | | | | | | | | | | | intervention should |
| accountability | | | | | | | | | | | | bring all unused study |
| | | | | | | | | | | | | intervention at each |
| | | | | | | | | | | | | clinic visit for tablet |
| | | | | | | | | | | | | counting. |
| Study intervention | | X | X | X | X | X | X | X | | | | Participants on study |
| dispensing | | | | | | | | | | | | intervention will be |
| | | | | | | | | | | | | provided with |
| | | | | | | | | | | | | sufficient study |
| | | | | | | | | | | | | intervention to last |
| | | | | | | | | | | | | until their next study |
| | | | | | | | | | | | | visit (except at |
| | | | | | | | | | | | | W 154 and ED). |

^{a}Adhoc visit is only applicable for LN patients who are receiving hydroxychloroquine: When patients are switched from 120 mg to 180 mg after Week 50, or when placebo patients who are on background therapy initiate ALXN2050 at any dose due to a Renal Flare, additional ECG needs to be obtained at an adhoc visit 2 weeks (±7 days) after the dose initiation or dose escalation.
^{b}For participants who discontinue study intervention prior to the end of the Open-label Extension Period, the ED Visit should be performed as soon as possible and a Safety Follow up Visit should be performed 30 days after the last dose of study intervention.
^{c}Renal Flare (as defined herein) and/or Severe Extrarenal SLE Flare (as defined herein) may occur at any time during the study. Evaluation of Renal Flare requires a UPCR from a spot urine sample that is confirmed on a 24-hour urine collection, as well as 2 serum creatinine samples obtained within a 2-week period. Evaluation of Renal and Extrarenal SLE Flares must be performed as soon as possible upon notification to the Investigator of symptom onset. If Renal Flare or Extrarenal SLE Flare occur between scheduled visits, only the assessments for the Renal Flare/Extrarenal SLE Flare Visit are needed. If Renal Flare or Extrarenal SLE Flare occur on a scheduled visit, all scheduled assessments should be performed for that visit as well as any additional assessments required for the evaluation of the flare.
^{d}Perform a symptom based neurologic examination if participant has complaints or clinical findings attributable to the CNS and if positive for findings, full neurologic examination to be performed at each assessment timepoint.
^{e}In the LN cohort, placebo patients will receive only background therapy. For all other patients in both cohorts, see FIGS. 1 and 2.
Note:
If possible, all assessments should be performed prior to administration of study intervention on in-clinic dosing days, unless otherwise specified.
Abbreviations: CNS = central nervous system; D = day; ED = early discontinuation; IgAN = immunoglobulin A nephropathy; LN = lupus nephritis; NPTP = nonpharmacologic therapies and procedures; PE = physical examination; SLE = systemic lupus erythematosus; UPCR = urine protein to creatinine ratio; W = week; WOCBP = women of childbearing potential.

### Objectives and Endpoints

The objectives and endpoints of this study are provided in Table 13 below.

TABLE 13
Mapping Objectives to Endpoints

| | |
|---|---|
| Objectives | Endpoints |

Primary (Both Cohorts)

| | |
|---|---|
| To evaluate the efficacy of ALXN2050 to | Percentage change in proteinuria from baseline to |
| reduce proteinuria in participants with LN or | Week 26 (based on 24-hour urine collection[s]) |
| IgAN | |

Secondary (Both Cohorts)

| | |
|---|---|
| To evaluate the efficacy of ALXN2050 to | Percentage change in proteinuria from baseline to |
| improve measures of kidney function in | Week 50 (based on 24-hour urine collection[s]) |
| participants with LN or IgAN | Achieving >30% and >50% reduction in proteinuria at |
| | Week 26 and Week 50 compared to baseline (based |
| | on 24-hour urine collection[s] at each time point) |
| | Change from baseline in eGFR at Week 26 and |
| | Week 50 |

Secondary (LN Cohort Only)

| | |
|---|---|
| To evaluate the efficacy of ALXN2050 on | Meeting the criteria for complete renal response |
| measures of kidney function in participants | (CRR) at Week 26 and Week 50 |
| with LN | Meeting the criteria for partial renal response (PRR) at |
| | Week 26 and Week 50 |
| | Time to the first occurrence of UPCR ≤0.5 g/g as |
| | measured by spot urine sample |
| | Achieving corticosteroid taper to 7.5 mg/day at |
| | Weeks 12, 26, and 50 |
| | Experience of a Renal Flare through Week 50 |
| | Experience of an Extrarenal systemic lupus |
| | erythematosus (SLE) Flare through Week 50 |
| | Meeting the criteria for treatment failure through |
| | Week 50 |
| | Meeting the criteria for Suboptimal Response through |
| | Week 50 |
| | Absolute values and change from baseline in serum |
| | albumin at Week 26 and Week 50 |

Secondary (IgAN Cohort Only)

| | |
|---|---|
| To evaluate the efficacy of ALXN2050 on | Meeting the criteria for partial remission at Week 26 |
| measures of kidney function in participants | and Week 50 |
| with IgAN | |

PK/PD (Both Cohorts)

| | |
|---|---|
| To characterize the pharmacokinetics (PK) | Observed plasma concentrations of ALXN2050 over |
| and pharmacodynamics (PD) of ALXN2050 in | time |
| participants with LN or IgAN | Absolute values and change from baseline in plasma |
| | Bb concentration and serum AP activity over time |

Safety (Both Cohorts)

| | |
|---|---|
| To characterize the safety and tolerability of | Incidence of TEAEs and TESAEs over time |
| ALXN2050 in participants with LN or IgAN | Changes from baseline in laboratory assessments |

Exploratory (Both Cohorts)

| | |
|---|---|
| To assess the efficacy of ALXN2050 in | Percentage change in proteinuria from baseline to |
| exploratory efficacy endpoints | Week 102 and Week 154 (based on 24-hour urine |
| | collection[s]) |
| | Change from baseline in eGFR at Week 102 and |
| | Week 154 |
| To evaluate the efficacy of ALXN2050 on | Effect on hematuria as measured by |
| hematuria in participants with LN or IgAN | Absolute value and change from baseline in |
| | RBC in urine from baseline to Week 26 and |
| | Week 50 |
| | Achieving <10 RBC/hpf |
| To assess quality of life based on | Change from baseline in SF-36 at Week 26 and |
| participant-reported outcomes in participants | Week 50 |
| with LN or IgAN based on treatment with | Change from baseline in EQ-5D-5L at Week 26 and |
| ALXN2050 | Week 50 |
| To evaluate biomarkers such as complement, | Absolute values and change from baseline in levels of |
| autoimmune, and renal in participants with LN | biomarkers in blood and urine over time |
| or IgAN | |

Exploratory (LN Cohort Only)

| | |
|---|---|
| To assess the efficacy of ALXN2050 in | Time to the first CRR or PRR (using spot UPCR) |
| exploratory efficacy endpoints | Meeting the criteria for Overall Renal Response (CRR |
| | or PRR) at Week 26 and Week 50 |
| | Time to the first occurrence of UPCR >50% decrease |
| | from baseline (using spot UPCR) |
| To assess quality of life based on | Change from baseline in FACIT-Fatigue total score at |
| participant-reported outcomes | Week 26 and Week 50 |
| To assess the efficacy of ALXN2050 in other | Histology changes from baseline to Week 50, if tissue |
| exploratory endpoints | is available |
| | Absolute values and change from baseline in anti- |
| | dsDNA and anti-C1q antibodies at Week 26 and |
| | Week 50 |

Exploratory (IgAN Cohort Only)

| | |
|---|---|
| To assess the efficacy of ALXN2050 in | Slope of eGFR computed from baseline to Week 26 |
| exploratory efficacy endpoints | and Week 50 |

Abbreviations:
AP = alternative pathway;
anti-C1q = antibodies against C1q complement component;
anti-dsDNA = antibodies against double-stranded DNA;
Bb = Bb fragment of complement factor B;
CRR = complete renal response;
eGFR = estimated glomerular filtration rate;
EQ-5D-5L = European Quality of Life Health 5-item questionnaire dimensions 5-level;
FACIT = Functional Assessment of Chronic Illness Therapy;
hpf = high-power field;
IgAN = immunoglobulin A nephropathy;
LN = lupus nephritis;
PD = pharmacodynamic(s);
PK = pharmacokinetic(s);
PRR = partial renal response;
RBC = red blood cell;
SLE = systemic lupus erythematosus;
TEAE = treatment emergent adverse event;
TESAE = treatment emergent serious adverse event;
SF-36 = Short Form 36 Health Survey;
UPCR = urine protein-to-creatinine ratio

### Justification for Dose

Clinical pharmacokinetics (PK) and PD data have been generated for ALXN2050 in single-ascending and multiple-ascending dose studies in healthy volunteers. In these clinical studies, ALXN2050 demonstrated a dose-proportional increase in systemic exposure following single-dose administration and a greater than dose-proportional increase following multiple-dose administration at steady state (Days 7 and 14) over the dosing range of 40 mg BID to 200 mg BID. Large intersubject variability was observed.

Following multiple-dose administration of ALXN2050 ranging from 40 mg BID to 200 mg BID in healthy participants, PD activity (determined by AP inhibition in the AP Wieslab assay) increased with increasing dose. The 120 mg BID dosing regimen provided sustained AP inhibition (AP activity <10%) in healthy participants whose ALXN2050 concentrations achieved the 90% inhibitory concentration (IC₉₀) threshold through the 12-hour dosing periods. Therefore, 120 mg BID is selected as the minimum therapeutic dose. Intersubject variability in PK and PK-PD relationship indicated that a dose higher than 120 mg BID (such as 180 mg BID) may be required to ensure more participants reach and maintain ALXN2050 concentration above the threshold for 90% AP inhibition. In addition, it is expected that FD baseline level will be elevated in patients who exhibit reduced kidney function, which would raise the ALXN2050 threshold concentration required for 90% inhibition of AP activity in patients with either LN or IgAN. Therefore, the 180 mg BID is selected as the likely therapeutic dose.

In a multiple ascending dose study, the 120 and 200 mg BID dose regimens were safe and effective, showing an approximately 10-fold or greater safety margin in both maximum plasma concentration (Cₘₐₓ) and the area under the concentration time curve from time zero to 24 hours (AUC₀₋₂₄) over the exposures achieved at the no observed adverse effect level (NOAEL) from nonclinical chronic toxicology studies (see Investigator's Brochure). In addition, both dosage regimens provided complete (>90%) and sustained inhibition of AP activity throughout the 12-hour dosing interval. Therefore, 120 mg BID is selected as the minimum therapeutic dosage.

Based on the favorable clinical safety and tolerability data from these studies, and the PK and PD characterization of ALXN2050, the dosing regimens of 120 mg BID and 180 mg BID are proposed for this dose-finding study in participants with LN or IgAN. The exposure range generated by the proposed ALXN2050 120 mg BID and 180 mg BID dosing is expected to be adequate to establish the PK-PD relationship in participants with LN or IgAN, thereby setting up the basis for dose selection in the planned Phase 3 study.

The optimal dose will be identified as the dose with the best benefit to risk ratio based on PK/PD modeling, safety, and efficacy data.

### Definitions of Study Periods and End of Study

The Screening Period is up to 6 weeks (Table 8 [LN cohort] and Table 9 [IgAN cohort]).

The blinded Initial Evaluation Period is from Day 1 to Week 26 (Table 8 [LN cohort] and Table 9 [IgAN cohort]).

The blinded Extended Treatment Period starts with dosing of study intervention at the Week 26 (Day 183) Visit and continues through the Week 50 (Day 351) Visit (Table 10 [LN cohort] and Table 11 [IgAN cohort]).

The OLE Period begins after the Week 50 Visit and continues up to 2 years (Table 12).

The Safety Follow-up Period is 4 weeks after the last dose of study intervention and includes a Safety Follow-up Visit 30 (±3) days after the last dose.

A participant is considered to have completed the study:
If he/she has completed all periods of the study, including the OLE Period and the last scheduled procedure shown in the SoA.
In the event the study is stopped early, the participant has completed all applicable periods of the study, including the ED and Safety Follow-up Visits.
The participant completes the study early because the study intervention is registered or approved (in accordance with country-specific regulations)

Early termination or discontinuation: A participant is considered to terminate early from the study if the participant is discontinued from the study during the blinded Initial Evaluation Period, blinded Extended Treatment Period, or OLE Period.

End of study: The end of study is defined as the date the last participant completes the last visit, including the OLE Period and Safety Follow-up Visit (Table 12).

### Study Population

### Inclusion Criteria

A participant must meet all inclusion criteria to be eligible to participate in the study.

### Age

1. Participant must be 18 and <75 years of age at the time of signing the informed consent.

### Sex

2. Male or female participant.
3. Female participants of childbearing potential and male participants must follow protocol-specified contraception guidance.

### Informed Consent

4. Capable of giving informed consent, which includes compliance with the requirements and restrictions listed in the informed consent form and in this protocol.

### Vaccinations and Antibiotics

5. Vaccinated against meningococcal infection (Neisseria meningitidis) within 3 years prior to, or at the time of randomization. Participants who initiate study intervention <2 weeks after receiving a meningococcal vaccine must receive appropriate prophylactic antibiotics until at least 2 weeks after the vaccination.

### Disease Characteristics

6. Local pathology report from the kidney biopsy used for eligibility must be available.

### Prior/Concomitant Therapy

7. Participants on sodium-glucose contransporter-2 (SGLT-2) inhibitors (e.g., empagliflozin) must be on a stable dose for 3 months with no planned change in dose during the Blinded Treatment Periods (through Week 50).

### Inclusion Criteria Specific for LN Cohort

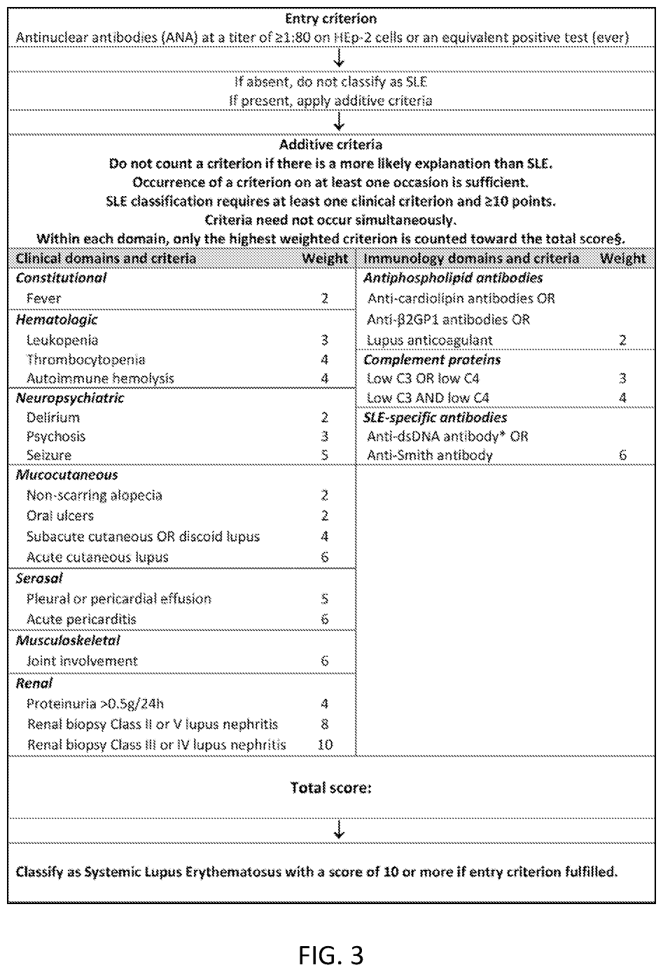

8. Clinical diagnosis of SLE by 2019 ACR and EULAR criteria (FIG. 3).
9. Diagnosis of 2018 Revised ISN/RPS classification (active focal or diffuse proliferative LN Class III or IV; Table 14]) confirmed by biopsy obtained <6 months prior to Screening or during Screening Period. Participants may co-exhibit Class V disease. See, e.g., Bajema et al. (Kidney Int. 2018; 93(4): 789-796), wherein Class V disease is characterized as glomerular capillary with numerous subepithelial immune deposits but no influx of leukocytes. Participants with de novo or relapsing disease may be eligible.

TABLE 14
Revised 2018 International Society of Nephrology/Renal
Pathology Society (ISN/RPS) Classification

| Category | Recommendation |
|---|---|
| Class II | Definition for mesangial hypercellularity adjusted: Four or more nuclei fully |
| | surrounded by matrix in the mesangial area not including the hilar region |
| Class III and IV | The term endocapillary proliferation is replaced by endocapillary |
| | hypercellularity |
| | The term crescent is used for a lesion consisting of extracapillary |
| | hypercellularity, composed of a variable mixture of cells. Fibrin and fibrous |
| | matrix may be present; 10% or more of the circumference of Bowman's |
| | capsule should be involved. |
| | Cellular crescent: more than 75% cells and fibrin and less than 25% fibrous |
| | matrix |
| | Fibrous crescent: more than 75% fibrous matrix and less than 25% cells and |
| | fibrin |
| | Fibrocellular crescent: 25% to 75% cells and fibrin and the remainder fibrous |
| | matrix |
| | Adhesion: an area of isolated continuity of extracellular matrix material |
| | between the tuft and capsule even when the underlying segment does not |
| | have overt sclerosis |
| | Fibrinoid necrosis: fibrin associated with glomerular basement membrane |
| | disruption and/or lysis of the mesangial matrix; this lesion does not require |
| | the presence of karyorrhexis |
| | Elimination of segmental and global subdivisions of Class IV |
| | Modification of the National Institutes of Health (NIH) lupus nephritis activity |
| | and chronicity scoring system to be used instead of the currently used A, C, |
| | and A/C parameters |
| Tubulointerstitial | Indicate whether interstitial inflammation occurs in presence or absence of |
| lesions | interstitial fibrosis |

Source: Bajema et al., Kidney Int. 2018; 93(4): 789-796.

10. Clinically active LN at Screening requiring/receiving immunosuppression induction treatment in the opinion of the Investigator.
11. Proteinuria with UPCR≥1 g/g based on one 24-hour urine collection during the Screening Period.

### Inclusion Criteria Specific for IgAN Cohort

12. Established diagnosis of primary IgAN based on kidney biopsy obtained any time prior to or during the Screening Period; the IgAN diagnosis classification criteria are provided in Table 15 below.

TABLE 15
2016 Oxford Classification from the IgA
Nephropathy Classification Working Group

| | |
|---|---|
| Detailed description | Light microscopy |
| of the features | Immunohistochemistry or immunofluorescence |
| present on: | Electron microscopy |
| Summary of 5 key | Mesangial score <0.5 (M0) or >0.5 (M1) |
| pathologic features | Endocapillary hypercellularity absent (E0) or |
| | present (E1) |
| | Segmental glomerulosclerosis absent (S0) or |
| | present (S1); presence or absence of podocyte |
| | hypertrophy/tip lesions in biopsy specimens with S1 |
| | Tubular atrophy/interstitial fibrosis ≤25% (T0), |
| | 526% to 0% (T1), or >50% (T2) |
| | Cellular/fibrocellular crescents absent (C0), |
| | present in at least 1 glomerulus (C1), in >25% |
| | of glomeruli (C2) |
| Quantitative data | Total number of glomeruli |
| | Number of glomeruli with endocapillary |
| | hypercellularity, necrosis, extracapillary |
| | hypercellularity (cellular/fibrocellular |
| | crescents), global glomerulosclerosis, and |
| | segmental glomerulosclerosis |

Source: Trimarchi et al., Kidney Int. 2017; 91(5); 1014-1021.

13. Mean proteinuria 1 g/day on 2 complete and valid 24-hour urine collections during the Screening Period.
14. For participants with a kidney biopsy performed >1 year prior to Screening that was used for eligibility:
   Presence of hematuria as defined by a positive result for blood on urine dipstick or 10 red blood cells (RBCs)/high-power field (hp) microscopy on urine sediment (documented by the local laboratory) during Screening Period. Presence of hematuria documented by the central laboratory may also be acceptable
15. Compliance with stable and optimal dose of RAS inhibitor treatment including maximum allowed or tolerated ACE inhibitor and/or ARB dose for 3 months prior to Screening with no expected change in dose during the Blinded Treatment Periods (through Week 50) (participants with established intolerance to RAS inhibitors may be included).
16. Controlled and stable blood pressure (defined as <140/90 mmHg) over the past 3 months prior to randomization.

### Exclusion Criteria

A participant will be excluded from the study if any of the following criteria apply.

### Disease Characteristics

1. Estimated GFR ≤30 mL/min/1.73 m² during Screening calculated by Chronic Kidney Disease Epidemiology Collaboration (CKD-EPI).
2. For participants with eGFR M 45 mL/min/1.73 m² at Screening, presence of any of the following in glomeruli on most recent kidney biopsy prior to or during the Screening Period:
   ≥50% interstitial fibrosis and tubular atrophy
   ≥glomerular sclerosis
   ≥active crescent formation
3. Concomitant significant renal disease other than LN or IgAN on the most recent biopsy prior to or during the Screening Period.
4. History of solid organ (kidney, heart, lung, small bowel, pancreas, or liver) or bone marrow transplant, or planned transplant during the blinded Extended Treatment Period (50 weeks).

### Medical Conditions

5. Splenectomy or functional asplenia.
6. History of seizure.
7. Known or suspected complement deficiency, unless attributable to underlying disease (i.e., LN and IgAN).
8. History or presence of any risk factors for Torsades de Pointes (e.g., heart failure/cardiomyopathy or family history of Long QT Syndrome), a screening QT interval corrected using Fridericia's formula (QTcF)>450 msec for males and >470 msec for females, or receiving medications known to significantly prolong the corrected QT interval (QTc), except for hydroxychloroquine in patients with LN.
9. Laboratory abnormalities at Screening, including:
   Alanine aminotransferase (ALT)>2× the upper limit of normal (ULN)
   Direct bilirubin >2×ULN
10. Hemoglobin A1C at Screening >7.0%,
11. Any other clinically significant laboratory abnormality that, in the opinion of the Investigator, would make the participant inappropriate for the study or put the participant at undue risk.
12. Institutionalization by administrative or court order or known medical or psychological condition(s) or risk factor that, in the opinion of the Investigator, might interfere with the participant's full participation in the study, pose any additional risk for the participant, or confound the assessment of the participant or outcome of the study.
13. Known or suspected history of drug or alcohol abuse or dependence within 1 year prior to the start of the Screening Period.
14. History of hypersensitivity to any ingredient contained in the study intervention, including inability to take or tolerate the standard of care background therapies, with the exception of RAS inhibitors for the IgAN cohort (Inclusion Criterion 15).
15. History of malignancy within 5 years prior to Screening with the exception of nonmelanoma skin cancer or carcinoma in situ of the cervix that has been treated with no evidence of recurrence.
16. Evidence of hepatitis B (positive hepatitis surface antigen [HBsAg] or positive core antibody (anti-HBc) with negative surface antibody [anti-HBs]) or hepatitis C viral infection (hepatitis C virus [HCV] antibody positive, except for patients with documented successful treatment and documented sustained virologic response [SVR]) at Screening.
17. Evidence of human immunodeficiency virus (HIV antibody positive) infection at Screening.
18. Bone marrow insufficiency with absolute neutrophil count <1.3×10³/μL; thrombocytopenia (platelet count <50,000/mm³).
19. Active systemic bacterial, viral, or fungal infection within 14 days prior to first dose of study intervention.
20. Presence of fever as documented by a temperature 38° C. (100.4° F.) within 7 days prior to administration of study intervention on Day 1.
21. History of Neisseria meningitidis infection.

### Prior/Concomitant Therapy

22. Current treatment with a biologic medication that may affect immune system functioning, or has stopped treatment with a biologic medication that may affect immune system functioning, and 5 terminal half-lives of the biologic medication have not elapsed by the time of the Screening Visit, or treatment with belimumab or rituximab <6 months prior to Screening.
23. Any previous or current treatment with complement inhibitors (e.g., eculizumab, ravulizumab).
24. Use of known cytochrome P450, family 3, subfamily A (CYP3A) sensitive substrates, moderate or strong CYP3A inducers, and/or moderate or strong CYP3A inhibitors from 2 weeks or 5 half-lives, whichever is longer, prior to the first administration of study intervention on Day 1 (randomization).
25. Use of selected medications known to lower the seizure threshold and/or cause seizure as disclosed herein.

### Prior/Concurrent Clinical Study Experience

26. Participation in another investigational drug or investigational device study within 30 days before initiation of study intervention on Day 1 in this study or within 5 half-lives of that investigational product, whichever is greater.

### Other Exclusions

27. Pregnant, breastfeeding, or intending to conceive during the course of the study.

### Exclusion Criteria Specific for LN Cohort

28. Participants who have received any of the following treatments for the current active LN flare:
   Cyclophosphamide ≤6 months prior to Screening
   Calcineurin inhibitors (CNIs)≤3 months prior to Screening
   A cumulative dose of intravenous (IV) methylprednisolone >3 g
   Mycophenolate mofetil (MMF)>2 g/day (or equivalent) for 4 consecutive weeks prior to Screening
   Prednisone or prednisone equivalent 0.5 mg/kg/day for 4 consecutive weeks prior to Screening
29. Uncontrolled hypertension (systolic blood pressure >160 mmHg or diastolic blood pressure >110 mmHg) on 2 or more measurements during the Screening Period.
30. Prior history or clinically active SLE-related cerebritis, seizures, pericarditis, stroke, or stroke syndrome requiring treatment.
31. Inability to take or tolerate the standard of care background therapies.

### Exclusion Criteria Specific for IgAN Cohort

32. Diagnosis of rapid progressive glomerulonephritis as measured by eGFR loss 30% over a period of 3 months prior to or during the Screening Period.
33. Secondary etiologies of IgAN (e.g., SLE, cirrhosis, celiac disease).
34. Clinically active Henoch-Schonlein purpura (IgA vasculitis) requiring treatment.
35. Prednisone or prednisone equivalent >20 mg/day for >14 consecutive days or any other systemic immunosuppression for the treatment of IgAN ≤6 months prior to Screening.
36. Blood pressure of ≥140/90 mmHg during the Screening Period confirmed on 2 measures >30 minutes apart.
37. Body mass index ≥38 kg/m² during Screening.

### Lifestyle Considerations

Certain foods such as grapefruit have been shown to be inhibitors of CYP3A4 enzyme activity. Participants should refrain from consuming these foods and beverages from 2 weeks prior to the first administration of study intervention on Day 1 until 2 weeks after the final dose of study intervention.

### Study Intervention

Study intervention is defined as any investigational intervention(s), marketed product(s), placebo, or medical device(s) intended to be administered to a study participant according to the study protocol.

### Study Invention(s) Administered

The interventions in this study are ALXN2050 and matching placebos. Study interventions are presented in Table 16 below. Background therapies for the LN and IgAN cohorts are as described herein.

TABLE 16
Study Interventions

| Study Intervention | | |
|---|---|---|
| Name | ALXN2050 | Placebo |
| Dose formulation | Tablet | Tablet |
| Physical description | Round, white to off- | Round, white to off- |
| | white film coated tablet | white film coated tablet |
| Unit dose | 60 mg | Placebo |
| strength(s) | | |
| Dosage levels | 120 mg BID or | Not applicable |
| | 180 mg BID | |
| Route of | Oral | Oral |
| administration | | |
| Use | Experimental | Placebo comparator |
| Former Name | ACH-0145228 | Not applicable |
| Sourcing | Alexion | Alexion |
| | Pharmaceuticals, Inc. | Pharmaceuticals, Inc. |

Participants will take study intervention BID (a dose of 3 tablets in the morning and a second [evening] dose of 3 tablets approximately 12 hours after the morning dose). Doses should be taken at approximately the same time each day. If a dose is missed, it should be taken within 6 hours of the originally scheduled time. After 6 hours, the missed dose should be skipped. In either case, the next dose should be taken according to the original dosing schedule. Information on missed doses should be recorded in the electronic case report form (eCRF).

Most doses will be taken outside of the clinic. Participants will be provided with sufficient study intervention to last until their next study visit.

For study visits that require PK sample collection, participants will be instructed to abstain from taking their ALXN2050 dose on the mornings of their study visits so that they can be dosed in the clinic following the protocol-required assessments. At the clinic visit, participants will first have blood drawn for clinical laboratory and other evaluations as outlined in the SoA (Tables 8-12), then take the morning dose of ALXN2050 assigned for that day.

### Blinded Treatment

ALXN2050 and placebo tablets will be identical in appearance. The blinded study interventions are shown in Table 17 below.

TABLE 17
Blinded Study Intervention from Week 1 to
Week 50 in Addition to Background Therapy

Dose Group

| | ALXN2050 | ALXN2050 | |
|---|---|---|---|
| Timing | 120 mg BID | 180 mg BID | Placebo |
| Morning dose | 60 mg | 60 mg | Placebo |
| | 60 mg | 60 mg | Placebo |
| | Placebo | 60 mg | Placebo |

Approximately 12 hours interval

| | | | |
|---|---|---|---|
| Evening dose | 60 mg | 60 mg | Placebo |
| | 60 mg | 60 mg | Placebo |
| | Placebo | 60 mg | Placebo |

Abbreviations:
BID = twice daily

### Prior and Concomitant Therapies

### Allowed Medication and Therapy

Any medication or therapy (including over-the-counter or prescription medicines, vaccines, vitamins, and/or herbal supplements) deemed necessary for the participant's care during the study, or for the treatment of any AE, along with any other medications, other than those listed as disallowed medications below, may be given at the discretion of the Investigator. However, it is the responsibility of the Investigator to ensure that details regarding the medication are recorded on the eCRF.

If adequate blood pressure control is not achieved during the study, participants may receive additional antihypertensive agents, but not agents that affect proteinuria during the study through Week 50. It is recommended that nonsteroidal anti-inflammatory drugs (NSAIDs) not be initiated during the study due to the possibility of adverse effects on renal function. However, this can used if indicated for symptomatic relief for a short period of time.

For participants in the LN cohort only:
Pneumocystis pneumonia prophylaxis is allowed at the discretion of the Investigator.
Treatment with antimalarial agents such as hydroxychloroquine are strongly recommended unless contraindicated.
Measures to prevent and treat osteoporosis are encouraged during the study; these measures may include any, or all, of the following: calcium carbonate or citrate, vitamin D, and bisphosphonates.
Immunosuppressive drugs for rescue therapy are allowed.

### Disallowed Medications and Therapy

Participants in both cohorts are prohibited from receiving any of the following medications and therapies during the entire duration of study participation:
Experimental interventions or therapies
A complement inhibitor (e.g., eculizumab, ravulizumab)
New use or modification of the dose of SGLT-2 inhibitors (up to 50 weeks)
New use or modification of the dose of RAS inhibitor or direct renin antagonist treatment (up to 50 weeks)
Known CYP3A sensitive substrates, moderate or strong CYP3A inducers, and/or moderate or strong CYP3A inhibitors are prohibited throughout the study, until 1 week after the final administration of study intervention (Table 18):

TABLE 18
List of Prohibited Inducers, Inhibitors, and Substrates of CYP3A

| Classification | Medication | Table Number^{a} |
|---|---|---|
| Strong CYP3A | boceprevir, cobicistat, danoprevir and ritonavir, | 3-2 |
| inhibitors | elvitegravir and ritonavir, grapefruit juice, indinavir and | |
| | ritonavir, itraconazole, ketoconazole, lopinavir and | |
| | ritonavir, paritaprevir and ritonavir and (ombitasvir | |
| | and/or dasabuvir), posaconazole, ritonavir, saquinavir | |
| | and ritonavir, telaprevir, tipranavir and ritonavir, | |
| | telithromycin, troleandomycin, voriconazole, | |
| | clarithromycin, idelalisib, nefazodone, and nelfinavir | |
| Moderate CYP3A | aprepitant, ciprofloxacin, conivaptan, crizotinib, | 3-2 |
| inhibitors | cyclosporine, diltiazem, dronedarone, erythromycin, | |
| | fluconazole, fluvoxamine, imatinib, tofisopam, and | |
| | verapamil | |
| Strong inducers of | apalutamide, carbamazepine, enzalutamide, mitotane, | 3-3 |
| CYP3A | phenytoin, rifampin, and St. John's wort | |
| Moderate inducers of | bosentan, efavirenz, etravirine, phenobarbital, and | 3-3 |
| CYP3A | primidone | |
| Sensitive substrates of | alfentanil, avanafil, buspirone, conivaptan, darifenacin, | 3-1 |
| CYP3A | darunavir, ebastine, everolimus, ibrutinib, lomitapide, | |
| | lovastatin, midazolam, naloxegol, nisoldipine, | |
| | saquinavir, simvastatin, sirolimus, tacrolimus, tipranavir, | |
| | triazolam, vardenafil, budesonide, dasatinib, | |
| | dronedarone, eletriptan, eplerenone, felodipine, | |
| | indinavir, lurasidone, maraviroc, quetiapine, sildenafil, | |
| | ticagrelor, tolvaptan | |

^{a}Table number from FDA Table of Clinical CYP Inhibitors and Inducers
Abbreviation:
CYP3A = cytochrome P450, family 3, subfamily A

Note: This list is complete as of 25 Jan. 2021.
Selected medications known to lower the seizure threshold and/or cause seizure should not be taken concomitantly:
   Meperidine/pethidine
   Tramadol
   Typical (first generation) antipsychotics
   Clozapine
   Olanzapine
   Lithium
   Tricyclic antidepressants
   Bupropion
   Aminophylline/theophylline
Medications known to significantly prolong the corrected QT interval (QTc), with the exception of hydroxychloroquine in patients with LN

In the event that a participant receives a prohibited medication and/or therapy, the Investigator will consider the discontinuation of study intervention. SGLT-2 inhibitors, RAS inhibitors, and direct renin antagonists are prohibited but may be considered without discontinuation of study intervention based on the discussion and approval of the Investigator and Medical Monitor.

Participants in the IgAN cohort are also prohibited from receiving any of the following medications and therapies during the entire duration of study participation:
Hydroxychloroquine
Immunosuppressive agents (e.g., MMF)
Systemic corticosteroids for >14 consecutive days (short-term steroid course for ≤14 days for medical conditions not related to IgAN or surgery are permitted)

For participants in the LN cohort, escalation of immunosuppression is allowed for protocol-defined Renal, Extrarenal Flares, and Suboptimal Response (after Week 26). Calcineurin inhibitors (CNIs) including, but not limited to voclosporin, are prohibited. If a CNI is used as rescue therapy per the clinical judgement of the Investigator, ALXN2050 will need to be discontinued 3 days prior to CNI administration.

However, the participant can remain in the study and continue the study visit as per the Schedule of Activities.

Any medications not specified in this section, that remain a concern to the Investigator should be discussed with the Medical Monitor.

### Background Therapies

The standard-of-care background therapies employed in this protocol are consistent with recent clinical studies in patients with LN (Rovin et al., Kidney Int. 2019; 95(1): 219-231) and IgAN (Rauen et al., N Engl J Med. 2015; 373(23): 2225-2236).

### Background Therapy for LN Cohort

During the course of the study, participants in the LN cohort will receive background therapy consistent with the standard of care for induction and maintenance treatment of LN.
For participants who have not started corticosteroid induction treatment prior to Screening:
   Participants will receive a cumulative dose of 1 g of methylprednisolone IV administered in 1 or multiple divided doses or oral equivalent during the Screening Period (prior to Day 1).
   During the Screening Period and no later than Day 2, all participants will receive oral corticosteroids with prednisone or prednisone equivalent with starting doses as outlined in Table 19. The starting minimum and maximum doses allowed are 30 mg/day and 60 mg/day, respectively. A corticosteroid taper will commence at Week 2 (Day 14). From Week 12 to Week 26, the target dose is 7.5 mg/day. Following Week 26, participants may remain on 7.5 mg/day or continue to taper at the clinical discretion of the Investigator until Week 46. From Week 46 to Week 50, the dose of corticosteroids must not be changed.
   During the Screening Period and no later than Day 1, participants will receive a cumulative dose of 1 to 1.5 g/day of MMF any time after completion of the IV methylprednisolone during the Screening Period and no later than Day 1. The dose can be administered in multiple divided doses. Participants will continue to receive 1 to 1.5 g/day for 1 week.
   After receiving 1 to 1.5 g/day for 1 week, the dose will be increased per the discretion of the Investigator to a cumulative dose of 2 to 3 g/day of MMF no later than by Week 4 (Day 28). The dose can be administered in multiple divided doses. Participants will continue to receive 2 to 3 g/day of MMF until Week 50 after which it may be decreased or discontinued based on the Investigators' judgment and the KDIGO clinical practice guidelines (KDIGO. Clinical Practice Guidelines for Glomerulonephritis. Kidney Disease Improving Global Outcomes. 2012; 2(2):209-217).
For participants who have initiated corticosteroid induction treatment prior to Screening and do not meet Exclusion Criterion 28:
   If the participant already received methylprednisolone IV ≥1 g or equivalent oral corticosteroid induction and is receiving MMF ≥2 g/day prior to Screening, then methylprednisolone IV will not be given. The participant may continue the current MMF dose during Screening, and during the Blinded Initial Evaluation Period the dose of MMF should be adjusted to achieve 2 to 3 g/day no later than Day 28 (Week 4). MMF will be continued at 2 to 3 g/day until Week 50 after which it may be decreased or discontinued based on the Investigator's judgment and the KDIGO clinical practice guidelines (KDIGO (2012), supra).
   If the participant already received methylprednisolone IV ≥1 g or equivalent oral corticosteroid induction and is receiving MMF <2 g/day, then methylprednisolone IV will not be given, and the MMF dose will be increased during the Screening Period (no later than Day 1) to a cumulative dose of 1 to 1.5 g/day. Participants will continue to receive 1 to 1.5 g/day for 1 week, after which the MMF dose will be increased per the discretion of the Investigator to 2 to 3 g/day to be achieved no later than Week 4 (Day 28). These doses can be administered in multiple divided doses. Participants will continue to receive 2 to 3 g/day until Week 50, after which it may be decreased or discontinued based on the Investigator's judgment and the KDIGO clinical practice guidelines (KDIGO (2012), supra).
   If a participant is already receiving prednisone or prednisone equivalent, the dose will be continued until Day 2, at which time prednisone or prednisone equivalent should be administered (the minimum and maximum doses allowed are 30 mg/day and 60 mg/day, respectively) as outlined in Table 19. The prednisone dose will be tapered starting at Week 2 (Day 14) according to the schedule. From Week 12 to Week 26, the target dose is 7.5 mg/day. Following Week 26, participants may remain on 7.5 mg/day or continue to taper at the clinical discretion of the Investigator until Week 46. From Week 46 to Week 50, the dose of corticosteroids must not be changed.

TABLE 19
Corticosteroid Taper for Participants with Lupus Nephritis

| | Prednisone Dose or Equivalent Dose (mg/day) | | | |
|---|---|---|---|---|
| Study week | ≤60 kg | 61 to 80 kg | 81 to 100 kg | ≥100 kg |
| Screening^{a} | 30 | 40 | 50 | 60 |
| to Week 2 | | | | |
| 2 | 25 | 35 | 40 | 50 |
| 4 | 25 | 30 | 30 | 40 |
| 6 | 20 | 25 | 20 | 30 |
| 8 | 15 | 20 | 15 | 20 |
| 10 | 10 | 15 | 10 | 10 |
| 12^{b} | 7.5 | 7.5 | 7.5 | 7.5 |

^{a}Weight at screening will be used for dosing and this weight category will be used all throughout the study
^{b}From Week 12 Week 26, the target dose is 7.5 mg/day. Following Week 26, participants may remain on 7.5 mg/day or continue to taper at the clinical discretion of the Investigator until Week 46. From Week 46 to Week 50, the dose of corticosteroids must not be changed.

An equivalent dose of enteric-coated mycophenolic acid sodium (MPS) may be used instead of MMF (i.e., 360 mg dose MPS is equivalent to a 500 mg dose of MMF).
Investigators may adjust the dosage of MMF due to tolerance or AEs. If the participant's symptoms resolve, the Investigator should attempt to increase MMF (or equivalent) to the goal level of 2 to 3 g/day. If symptoms return, then the participant should be continued on the highest tolerable dose.
Changes to the dose of MMF and the justification will be documented in the eCRF.
   Other considerations regarding the corticosteroid taper:
All participants will have a scheduled corticosteroid taper starting on Day 14. Participants will reduce their prednisone dose according to their baseline body weight over 10 weeks until the dose is 7.5 mg/day by Week 12 (Table 19). From Week 12 to Week 26, the target dose is 7.5 mg/day. Following Week 26, participants may remain on 7.5 mg/day or continue to taper at the clinical discretion of the Investigator until Week 46. From Week 46 to Week 50, the dose of corticosteroids must not be changed.
Deviations from the scheduled corticosteroid taper for any reason other than Renal Flare or Extrarenal SLE Flare will confound interpretation, so every attempt should be made to adhere to the tapering schedule.
If disease is too clinically active in the opinion of the Investigator to begin the corticosteroid taper after Week 2, then the participant may continue to receive his or her initial corticosteroid dose for up to an additional 28 days. Similarly, participants who have started the taper and whose disease is too clinically active to continue tapering may remain at the same taper dose achieved for up to an additional 28 days. Failure to achieve the corticosteroid taper by Week 12 will not be considered as Treatment Failure and will be captured as a secondary endpoint.
However, the prednisone dose may NOT be increased beyond the taper dose achieved unless the participant meets the protocol-defined criteria for Renal Flare and/or severe Extrarenal SLE Flare, in which case these participants will receive rescue therapy and will be included as Treatment Failures.

### Background Therapy for IgAN Cohort

The background therapies for participants in the IgAN cohort will be consistent with standard of care and include the maximumly tolerated dose of RAS-blocking agents, such as ACE inhibitors or ARBs. The background treatment should be held stable throughout the first 50 weeks of the Treatment Period of the study.

### Rescue Therapy for LN Cohort

In the LN cohort, participants who meet criteria for protocol-defined Renal Flare will receive rescue therapy.

Participants in the LN cohort who meet the criteria for Extrarenal SLE Flare may receive rescue therapy, if considered clinically appropriate by the Investigator. If rescue therapy is administered for an Extrarenal SLE Flare, the event is considered a severe Extrarenal SLE Flare.

After Week 26, rescue therapy for participants with Suboptimal Response is allowed at the clinical discretion of the Investigator in conversation with the Medical Monitor.

Rescue therapy, all flares, and Suboptimal Response will be documented in the eCRF.

Rescue Therapy is defined as intensification of current standard of care or introduction of new immunosuppressive therapies. The specific choice of Rescue Therapy(ies) is generally at the discretion of the Investigator and may include approved medications for LN (e.g., voclosporin, belimumab).

However, if a CNI is used as rescue therapy, ALXN2050 will need to be discontinued 3 days prior to CNI administration.

The following guidelines for corticosteroid dosing for protocol-defined Renal Flare and Extrarenal SLE Flare should be considered to maintain treatment consistency:
Participants with protocol-defined Renal Flare may be treated with oral prednisone up to 0.5 mg/kg/day (not to exceed 60 mg/day) for up to 2 weeks. Prednisone will then be tapered weekly to 10 mg/day within 6 weeks after the initial prednisone increase. Prednisone may further be tapered to ≤7.5 mg/day at the discretion of the Investigator.
Participants with Extrarenal SLE Flare may be treated with oral prednisone up to 1 mg/kg/day (not to exceed 60 mg/day) for up to 2 weeks. Prednisone will then be tapered every 2 weeks to achieve 7.5 mg/day within 12 weeks after the initial corticosteroid increase.
Intravenous corticosteroids in equivalent doses may be allowed if gastrointestinal involvement temporality precludes oral corticosteroid use.

Prednisone ≥10 mg for ≤14 days will not be considered rescue therapy in the following instances:
Renal Flares not meeting the protocol-defined criteria for Renal Flare.
Other medical conditions or surgery

The use of rescue therapy should be discussed directly between the Investigator and Medical Monitor.

### Vaccination and Antibiotic Prophylaxis

To mitigate the potential risk of meningococcal infection, all participants must be vaccinated within 3 years prior to, or at the time of, initiating the study intervention. Vaccines against serotypes A, C, Y, W135, and B, where available, are recommended to prevent common pathogenic meningococcal serotypes. Participants who initiate study intervention treatment less than 2 weeks after receiving a meningococcal vaccine must receive treatment with appropriate prophylactic antibiotics until at least 2 weeks after vaccination.

Participants must be vaccinated or revaccinated according to current national vaccination guidelines or local practice for vaccination use with complement inhibitors. Vaccination may not be sufficient to prevent meningococcal infection. All participants should be monitored for early signs of meningococcal infection, evaluated immediately if infection is suspected, and treated with appropriate antibiotics, if necessary.

Any participant without sufficient history of these vaccines may be vaccinated or provided boosters per national or local guidelines.

Given the chronic progression of IgAN, in participants with IgAN, every effort should be made to start the meningococcal vaccination series at least 14 days prior to randomization.

Participants should be vaccinated or revaccinated against other pathogens according to current national vaccination guidelines or local practice for vaccination use as part of standard of care.

### Dose Modification

Once the optimal dose of ALXN2050 is determined, participants on ALXN2050 who have completed at least 50 weeks of treatment will be switched to the optimal dose as indicated in FIG. 1 (for LN cohort) and FIG. 2 (for IgAN).

### Study Assessments and Procedures

### Efficacy Assessment

### 24-Hour Urine Collection for Proteinuria

For the determination of proteinuria, 24-hour urine collections will be obtained at the time points specified in the SoA (Tables 8-12) and will be analyzed by a central laboratory. In addition to protein, albumin, sodium, and creatinine will be quantified in each 24-hour urine collection. Both UPCR as well as urine albumin to creatinine ratio (UACR) will be calculated using an aliquot of the 24-hour urine collections.

Rigorous exercise and significant change in diet (in particular, salt intake) should be avoided within 48 hours before collection of 24-hour urine samples, whenever possible.

The collection should be obtained prior to or >7 days after biopsy procedures and prior to administration of study intervention on dosing days.

The 24-hour urine collections will occur at the participant's home by a mobile burse if agreed upon with the Investigator and the participant, according to local regulations. The collection will be recorded in the eCRF according to the eCRF completion guidelines.

### LN Cohort

For participants in the LN cohort, proteinuria will be measured by UPCR. A single 24-hour urine collection will be obtained at Screening to assess eligibility. Two separate 24-hour urine collections will be obtained within 2 weeks prior to the Week 26 Visit (to assess the primary endpoint) and Week 50 Visit (to assess a secondary endpoint). Additional 24-hour urine collection is also scheduled for Week 102 and Week 154.

Confirmation of a protocol-defined Renal Flare or Suboptimal Response requires a single 24-hour urine collection within 2 weeks of the spot urine sample.

### IgAN Cohort

Participants in the IgAN cohort will be required to provide 2 separate, complete, and valid 24-hour urine collections during the Screening Period (to assess eligibility). For evaluation of the primary and key secondary endpoints the two 24-hour urine collections should be obtained within 2 weeks before the Week 26 and Week 50 visits. Additional 24-hour urine collection is also scheduled for Week 102 and Week 154. Hence, a 24-hour urine collection is considered valid if all the following criteria are met, otherwise the urine collection is required to be repeated:
The collection is between 22 to 26 hours in duration (i.e., time from the initial discarded void to the last void/attempt to void).
No voids are missed between the start and end time of the collection as indicated by the participant's urine collection diary.

Urine collections will be reviewed by the Medical Monitoring team.

Inadequate collections may need to be repeated as soon as possible within the time frames outlined in the Schedule of Activities in order to ensure that 2 valid collections are obtained for each of the study time points.

### Spot Urine Sample (Both Cohorts)

Urinary protein, albumin, and creatinine levels from morning spot urine samples prior to dosing will be measured at the time points specified in the SoA (Tables 8-12) to assess the effect of study intervention on UPCR and UACR.

Two consecutive spot urine samples will be obtained for participants in both disease cohorts at Week 16 for the LN cohort and at Week 18 for the IgAN cohort.

The UPCR and UACR results will be recorded in the participant's eCRF.

### Estimated Glomerular Filtration Rate (Both Cohorts)

Changes in renal function will be monitored using measurements of eGFR (mL/min/1.73 m²) and creatinine clearance on a 24-hour urine collection. Serum creatine will be analyzed as part of the clinical chemistry collected predose. The eGFR calculation will be based on the CKD-EPI formula.

For the determination of CRR and PRR at Week 26 and Week 50, 2 serum creatinine samples for eGFR will be obtained, the first within 2 weeks prior to each of these study visits and the second on the study visit day. The blood sample collection could occur at the participant's home if agreed upon with the Investigator and the participant. The collection will be recorded in the eCRF according to the eCRF completion guidelines.

### Hematuria (Both Cohorts)

For participants in both disease cohorts, hematuria from spot urine samples will be evaluated to assess the effect of study intervention on disease course. The degree of hematuria will be assessed by examination of the spun urine sediment by microscopy (RBC/hpf).

Single void collections for random spot urine sample for hematuria evaluation should be collected. If the Investigator determines that the hematuria is transient due to menses in women or exercise, the sample may need to be repeated.

Random spot urine samples for hematuria measurement will be collected throughout the study as outlined in the SoA (Tables 8-12) and will be analyzed by a central laboratory. Samples should be collected prior to study intervention administration, if applicable.

The local hematuria evaluation by microscopy or urinary dipstick may be used to determine eligibility for the study at Screening for participants with IgAN, if diagnostic biopsy is >2 years.

### Albumin (LN Cohort Only)

For renal function assessment, samples for serum albumin will be collected as part of the clinical chemistry evaluations.

### Renal Flare (LN Cohort Only)

Renal Flare is determined in the opinion of the Investigator in addition to the criteria outlined below:
For participants who achieve CRR, a Renal Flare is the reproducible recurrence of proteinuria ≥1 g/g
For all other participants, a Renal Flare is either of the following:
   Reproducible increase of serum creatinine >25% higher than baseline or above the ULN, plus any one of the following:
      Reproducible proteinuria ≥75% higher than baseline
      Worsening active urinary sediment compared to baseline as defined by an increase of ≥5 RBCs/hpf or new RBC casts (based on local laboratory results from at least 2 samples)
      Kidney biopsy demonstrating LN Class III or IV activity which was conducted since the biopsy used for eligibility
   Reproducible doubling of the UPCR from a 24-hour urine collection compared with the lowest previous value obtained after the first dose of study intervention.
Reproducibility of proteinuria requires that the proteinuria based on a UPCR from a morning spot urine collection is confirmed by UPCR calculated on a 24-hour urine collection obtained within a 2-week period.
Reproducibility of serum creatinine requires 2 blood tests within a 2-week period.

Participants who meet criteria for the protocol-defined Renal Flare will receive rescue therapy. The Medical Monitor should be notified of the Renal Flare by the Investigator or Sub-investigator.

Any Renal Flare that does not meet the protocol-defined Renal Flare criteria may be treated with a limited duration of increased oral corticosteroids (<14 days) after discussion with the Medical Monitor.

Such treatment will not be considered rescue therapy and will not be considered Treatment Failure.

Renal Flare criteria will be recorded on the Renal Flare eCRF.

### Extrarenal Systemic Lupus Erythematosus Flare (LN Cohort Only)

The Systemic Lupus Erythematosus Disease Activity Index 2000 (SLEDAI-2K) is an instrument that assesses the disease activity of SLE. This instrument will be used for the monitoring of Extrarenal SLE Flare in the LN Cohort.

Extrarenal SLE Flare is defined as an increase in SLEDAI-2K Safety of Estrogens in Lupus Erythematosus National Assessment (SELENA) Modification 4 points that is not accounted for by proteinuria, hematuria, urinary cellular casts, hypocomplementemia, or an increase in anti-double-stranded DNA (anti-dsDNA) antibody level.

Participants in the LN cohort who meet the criteria for Extrarenal SLE Flare may receive Rescue Therapy, if considered clinically appropriate by the Investigator. If Rescue Therapy is administered, the event is considered a severe Extrarenal SLE Flare.

Participants will be allowed to receive a limited number of corticosteroid treatments for non-severe Extrarenal SLE Flare, if clinically warranted as outlined below. Such treatment will not be considered Rescue Therapy and will not be considered Treatment Failure.
Up to 2 corticosteroid treatments will be allowed for a non-severe Extrarenal SLE Flare.
One corticosteroid treatment for a non-severe Extrarenal SLE Flare will be allowed between Week 12 (end of steroid taper) and Week 22 (4 weeks prior to the Week 26 primary endpoint).
One corticosteroid treatment will be allowed between Week 26 and Week 46 (4 weeks prior to the Week 50 analysis).
Each treatment course should be no longer than 14 days in duration with the steroid dose returning to <7.5 mg/day by Day 14.
Up to a total of 20 mg/day (prednisone or prednisone equivalent) is permitted.
There can be no concurrent worsening of renal disease (as defined by the criteria for a Renal Flare).

### Suboptimal Response (LN Cohort Only)

A Suboptimal Response is determined in the opinion of the Investigator in addition to the following criterion after the Week 26 Visit:
Reproducible proteinuria ≤25% decreased compared to baseline based on UPCR on a 24-hour urine collection performed by central laboratory

Reproducibility of proteinuria requires that the proteinuria based on a UPCR from a spot urine collection is confirmed by a central laboratory UPCR calculated on a 24-hour urine collection obtained within a 2-week period.

Participants with Suboptimal Response must be discussed with the Investigator and Medical Monitor. Participants with Suboptimal Response will stay in the study and continue to receive study intervention. Intensification of current standard of care or introduction of new immunosuppressive therapies are allowed per the clinical discretion of the Investigator in conversation with the Medical Monitor and will be considered Rescue Therapy.

Participants with Suboptimal Response will be included as Treatment Failure.

### Treatment Failure (LN Cohort Only)

Treatment Failure is defined as the receipt of Rescue Therapy at any time during the study for protocol defined Renal Flare, Extrarenal SLE Flare, or Suboptimal Response.

Increase in corticosteroids for Extrarenal SLE Flare not meeting the protocol definition of Severe Extrarenal SLE Flare, Renal Flare not meeting protocol definition for Renal Flare, lack of response not meeting the protocol definition of Suboptimal Response, other medical conditions, or surgery limited to ≤14 days duration are not included in Treatment Failure.

Participants who meet the criteria for Treatment Failure may continue to receive the study intervention and stay in the study. If a participant has 2 Treatment Failures, study intervention discontinuation should be considered. If a CNI is used for rescue therapy, study intervention will need to be discontinued 3 days prior to CNI administration.

### Complete and Partial Renal Response (LN Cohort Only)

Complete Renal Response and PRR will be assessed at Week 26 and Week 50.

To achieve CRR (Rovin (2019), supra), participants in the LN cohort must meet all 3 of the following criteria:
A decrease in mean UPCR to ≤0.5 g/g based on two 24-hour urine collections obtained within 2 weeks prior to the study visit (Week 26 or Week 50)
Estimated glomerular filtration rate >60 mL/min/1.73 m² or no eGFR reduction 20% from the baseline value based on the mean of 2 values. The first eGFR value must be obtained within 2 weeks prior to the study visit (Week 26 or Week 50) and the second eGFR value will be obtained on the study visit (Week 26 or Week 50).
No Treatment Failure (see above)

To achieve PRR (Rovin (2019), supra), participants who did not achieve CRR in the LN cohort must meet all 3 of the following criteria:
A decrease in UPCR≥50% compared to the baseline value based on mean of two 24-hour urine collections obtained within 2 weeks prior to the study visit (Week 26 or Week 50)
Estimated glomerular filtration rate >60 mL/min/1.73 m² or no eGFR reduction 20% from the baseline value based on the mean of 2 values. The first eGFR value must be obtained within 2 weeks prior to the study visit (Week 26 or Week 50) and the second eGFR value will be obtained on the study visit (Week 26 or Week 50)
No Treatment Failure (see above)

Overall Renal Response is defined as the composite of CRR and PRR.

### Partial Remission (IgAN Cohort Only)

Partial Remission will be defined as mean proteinuria <1 g/24 hours based on 2 valid 24-hour urine collections obtained within 2 weeks prior to the study visit (Week 26 or Week 50).

### Participant-Reported Outcomes

The following participant-reported outcome (PRO) instruments will be used in this study used to capture health-related quality of life (QoL):
European Quality of Life Health 5-item questionnaire dimensions 5 level (EQ-5D-5L for both cohorts)
Short Form (SF)-36 Health Survey (for both cohorts)
Functional Assessment of Chronic Illness Therapy (FACIT)-Fatigue scale (for LN cohort only)

All instruments will be self-reported and administered at visits specified in the SoAs. Information about these instruments and questionnaires, with the correct version and the validated language versions, if needed, will be provided to the sites prior to the start of the study.

### EQ-5D-5L (for Both Cohorts)

The EQ-5D-5L is a self-reported standardized instrument to measure health related QoL and has been used in a wide range of health conditions. The EQ-5D-5L is defined by 5 dimensions: mobility, usual activities, self-care, pain/discomfort, and anxiety/depression. A 0 to 1 health state index score (or utility score), where 0 indicates a health state equivalent to death and 1 indicates perfect health, will be calculated from individual health profiles using a US TTO value set. Negative values indicate health states considered worse than death. Each of the EQ-5D-5L dimensions may be summarized and analyzed as a categorical variable, providing data on the health profile of the study patients. The visual analog scale (VAS) and EQ-5D-5L index score may be summarized and analyzed as continuous variables. The instrument will be used in both cohorts.

### SF-36 (for Both Cohorts)

The SF-36 is a set of generic, coherent, and easily administered quality-of-life measures. It has 36 items grouped in 8 dimensions: Physical Functioning, Physical, Bodily Pain, Vitality, General Health, Emotion, Mental Health, and Social Functioning. This instrument will be used in both cohorts.

### FACIT-Fatigue (for LN Cohort Only)

The FACIT-Fatigue scale is a collection of QoL questions pertaining to the management of fatigue symptoms due to a chronic illness. The FACIT-Fatigue Scale (Version 4) is a short, 13-item, self-reported, easy to administer tool that measures an individual's level of fatigue during their usual daily activities over the past week. The level of fatigue is measured on a five-point Likert-type scale (0=not at all fatigued; 1=a little bit fatigued, 2=somewhat fatigued, 3=quite a bit fatigued, and 4=very much fatigued). All items contribute to the sum score with equal weight. A score of less than 30 represents severe fatigue and higher scores indicate improvement in fatigue. A 3-point change is clinically meaningful on this scale. A 10-point or greater change is highly significant on this scale. This instrument will be used in the LN cohort.

### OTHER EMBODIMENTS

While the disclosure describes specific embodiments of methods, compounds, compositions, and uses, It will be understood that further modifications can be made thereto, and this application is intended to cover any variations or adaptations thereof following, in general, the principles of the disclosure including such departures from the disclosure that come within known or customary practice within the art to which the disclosure pertains and may be applied to essential features hereinbefore set forth, and follows in the scope of the claims. Other embodiments are within the claims.

1. A method of treatment, wherein the method comprises treating lupus nephritis (LN) and/or immunoglobulin A nephropathy (IgAN) in a subject, said treating comprising administering to the subject a therapeutically effective amount of Compound 1: 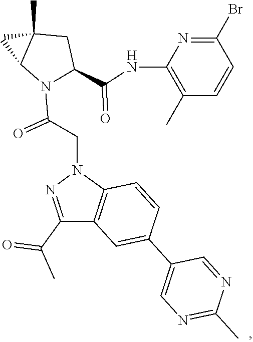 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof and reducing proteinuria in the subject from baseline.

2. The method of claim 1, wherein Compound 1 or the pharmaceutically acceptable salt thereof is administered at a dose of about 60 mg to about 300 mg twice daily (BID).

3. The method of claim 2, wherein Compound 1 or the pharmaceutically acceptable salt thereof is administered at a dose of about 120 mg BID.

4. The method of claim 3, wherein Compound 1 or the pharmaceutically acceptable salt thereof is administered at a dose of about 180 mg BID.

5. The method of any one of claims 1-4, wherein said treating comprises reducing proteinuria in the subject from baseline following a 50-week treatment period.

6. The method of claim 5, wherein said treating comprises reducing proteinuria in the subject by greater than about 30% from baseline following a 50-week treatment period.

7. The method of claim 6, wherein said treating comprises reducing proteinuria in the subject by greater than about 50% from baseline following a 50-week treatment period.

8. The method of any one of claims 1-7, wherein said treating comprises reducing proteinuria in the subject from baseline following a 26-week treatment period.

9. The method of claim 8, wherein said treating comprises reducing proteinuria in the subject by greater than about 30% from baseline following a 26-week treatment period.

10. The method of claim 8, wherein said treating comprises reducing proteinuria in the subject by greater than about 50% from baseline following a 26-week treatment period.

11. The method of any one of claims 1-10, wherein said treating further comprises improving renal function in the subject.

12. The method of claim 11, wherein said improving renal function comprises increasing an estimated glomerular filtration rate (eGFR) from baseline in the subject after a 50-week treatment period as calculated using Chronic Kidney Disease Epidemiology Collaboration (CKD-EPI).

13. The method of claim 11 or 12, wherein said improving renal function comprises increasing an eGFR from baseline in the subject after a 26-week treatment period as calculated using CKD-EPI.

14. The method of claim 11, wherein said improving renal function comprises reducing an increase in an eGFR from baseline in the subject relative to a control after a 50-week treatment period, as calculated using CKD-EPI.

15. The method of claim 11 or 14, wherein said improving renal function comprises reducing an increase in an eGFR from baseline in the subject relative to a control after a 26-week treatment period, as calculated using CKD-EPI.

16. The method of any one of claims 11-15, wherein said improving renal function comprises improving creatinine clearance in the subject.

17. The method of any one of claims 1-16, wherein the subject has LN.

18. The method of claim 17, wherein the subject has been diagnosed with active focal or diffuse proliferative LN class II or IV confirmed by a kidney biopsy obtained <6 months prior to treatment.

19. The method of claim 18, wherein the subject is exhibiting Class V disease.

20. The method of any one of claims 17-19, wherein the LN is de novo LN.

21. The method of any one of claims 17-19, wherein the LN is relapsing LN.

22. The method of any one of claims 17-21, wherein the LN is clinically active LN which requires immunosuppression induction treatment.

23. The method of any one of claims 17-22, wherein the subject has proteinuria with urine protein:creatinine ratio (UPCR)≥1 g/g based on a 24-hour urine collection prior to treatment.

24. The method of claim 23, wherein the time to first occurrence of UPCR≤0.5 g/g as measured by spot urine sample is reduced as compared to a control.

25. The method of any one of claims 17-24, wherein the subject experiences partial renal response (PRR) following a 50-week treatment period.

26. The method of any one of claims 17-25, wherein the subject experiences complete renal response (CRR) following a 50-week treatment period.

27. The method of any one of claims 17-26, wherein the subject experiences PRR following a 26-week treatment period.

28. The method of any one of claims 17-27, wherein the subject experiences CRR following a 26-week treatment period.

29. The method of any one of claims 17-28, wherein said treating comprises reducing a time to first occurrence of UPCR≤0.5 g/g from baseline as measured by spot urine sample, as compared to a control.

30. The method of any one of claims 17-29, wherein the subject has not started corticosteroid induction treatment prior to the treatment with Compound 1 or the pharmaceutically acceptable salt thereof.

31. The method of claim 30, wherein the subject is administered a cumulative dose of about 1 g of methylprednisolone IV in one or multiple divided doses prior to the start of treatment with Compound 1 or the pharmaceutically acceptable salt thereof.

32. The method of claim 30 or 31, wherein the subject is administered an oral corticosteroid at a dose of 0.5 mg/kg/day with a minimum dose of about 30 mg/day and a maximum dose of about 60 mg/day prior to the start of treatment with Compound 1 or the pharmaceutically acceptable salt thereof.

33. The method of claim 30 or 31, wherein the subject said treating comprises co-administering an oral corticosteroid at a dose of about 30 mg/day to about 60 mg/day.

34. The method of claim 33, wherein the dose of the oral corticosteroid is tapered to 7.5 mg/day after a 50-week treatment period.

35. The method of claim 33 or 34, wherein the dose of the oral corticosteroid is tapered to 7.5 mg/day after a 26-week treatment period.

36. The method of any one of claims 33-35, wherein the dose of the oral corticosteroid is tapered to 7.5 mg/day after a 12-week treatment period.

37. The method of any one of claims 31-36, wherein the subject is administered a first dose of mycophenolate mofetil (MMF) about 1-1.5 g/day in one or more doses after administration of the cumulative dose of about 1 g of methylprednisolone IV prior to treatment with Compound 1 or the pharmaceutically acceptable salt thereof, and is administered a second dose of MMF in one or more doses until 50 weeks after the start of treatment with Compound 1 or the pharmaceutically acceptable salt thereof.

38. The method of claim 37, wherein the second dose is about 1-1.5 g/day.

39. The method of claim 37, wherein the second dose is about 1-1.5 g/day until 1 week after the start of treatment with Compound 1 or the pharmaceutically acceptable salt thereof, after which the second dose is increased to about 2-3 g/day.

40. The method of any one of claims 17-29, wherein the subject has initiated corticosteroid induction treatment prior to the start of treatment with Compound 1 or the pharmaceutically acceptable salt thereof.

41. The method of claim 40, wherein the subject has received a cumulative dose of methylprednisolone IV of about 1 g or an equivalent oral corticosteroid and is receiving a first dose of MMF of about 2 g/day in one or more doses prior to the start of treatment with Compound 1 or the pharmaceutically acceptable salt thereof, the subject is not administered an additional dose of methylprednisolone IV or equivalent oral corticosteroid, and the subject is administered a second dose of MMF in one or more doses until 50 weeks after the start of treatment with Compound 1 or the pharmaceutically acceptable salt thereof.

42. The method of claim 41, wherein the second dose of MMF is ≥about 2 g/day.

43. The method of claim 41, wherein the second dose of MMF is ≥about 2 g/day, and is adjusted to about 2-3 g/day before 4 weeks after the start of treatment with Compound 1 or the pharmaceutically acceptable salt thereof.

44. The method of claim 40, wherein the subject has received a cumulative dose of methylprednisolone IV of ≥about 1 g or an equivalent oral corticosteroid and is receiving a first dose of MMF of <about 2 g/day in one or more doses prior to the start of treatment with Compound 1 or the pharmaceutically acceptable salt thereof, and the subject is not administered an additional dose of methylprednisolone IV or equivalent oral corticosteroid, and the subject is administered a second dose of MMF in one or more doses until 50 weeks after the start of treatment with Compound 1 or the pharmaceutically acceptable salt thereof.

45. The method of claim 44, wherein the second dose of MMF is about 1-1.5 g/day at the start of treatment with Compound 1 or the pharmaceutically acceptable salt thereof.

46. The method of claim 45, wherein the second dose of MMF is about 1-1.5 g/day for a one-week period following the start of treatment with Compound 1 or the pharmaceutically acceptable salt thereof, after which the second dose of MMF is increased to about 2-3 g/day before 4 weeks after the start of treatment with Compound 1 or the pharmaceutically acceptable salt thereof.

47. The method of any one of claims 40-46, wherein the subject has been receiving prednisone or a prednisone equivalent at a first dose prior to the start of treatment with Compound 1 or the pharmaceutically acceptable salt thereof, the first dose is maintained through the second day of treatment with Compound 1 or the pharmaceutically acceptable salt thereof, after which the subject is administered an oral corticosteroid at a dose of 0.5 mg/kg/day with a minimum dose of about 30 mg/day and a maximum dose of about 60 mg/day.

48. The method of claim 47, wherein the dose of the oral corticosteroid is tapered to 7.5 mg/day after a 50-week treatment period.

49. The method of claim 47 or 48, wherein the dose of the oral corticosteroid is tapered to 7.5 mg/day after a 26-week treatment period.

50. The method of any one of claims 47-49, wherein the dose of the oral corticosteroid is tapered to 7.5 mg/day after a 12-week treatment period.

51. The method of any one of claims 17-50, wherein said treating comprises reducing a risk of experiencing a renal flare in the subject within a 50-week treatment period.

52. The method of any one of claims 17-51, wherein said treating comprises reducing a risk of experiencing an extrarenal systemic lupus erythematosus (SLE) flare in the subject within a 50-week treatment period.

53. The method of any one of claims 17-52, wherein said treating comprises reducing a risk of treatment failure in the subject within a 50-week treatment period.

54. The method of any one of claims 17-53, wherein said treating comprises reducing a level of serum albumin in the subject from baseline.

55. The method of any one of claims 17-54, wherein said treating comprises reducing the time to first CRR or PRR as compared to a control.

56. The method of any one of claims 17-54, wherein said treating comprises reducing a time to first occurrence of UPCR>50% decrease from baseline as compared to a control.

57. The method of any one of claims 17-56, wherein the subject exhibits an increase in FACIT-Fatigue total score from baseline following a 50-week treatment period.

58. The method of any one of claims 17-57, wherein the subject exhibits an increase in FACIT-Fatigue total score from baseline following a 26-week treatment period.

59. The method of any one of claims 17-58, wherein a level of antibodies against double-stranded DNA (anti-dsDNA) and/or antibodies against C1q complement component (anti-C1q) in the subject is reduced from baseline following a 50-week treatment period.

60. The method of any one of claims 17-59, wherein a level of anti-dsDNA and/or anti-C1q in the subject is reduced from baseline following a 26-week treatment period.

61. The method of any one of claims 17-59, wherein the subject has not received treatment with cyclophosphamide ≤6 months prior to treatment with Compound 1 or the pharmaceutically acceptable salt thereof.

62. The method of any one of claims 17-61, wherein the subject has not received treatment with a calcineurin inhibitor ≤3 months prior to treatment with Compound 1 or the pharmaceutically acceptable salt thereof.

63. The method of any one of claims 17-62, wherein the subject has not received treatment with a cumulative dose of intravenous (IV) methylprednisolone >about 3 g for active renal flare.

64. The method of any one of claims 17-63, wherein the subject has not received treatment with MMF >about 2 g/day or an equivalent thereof for 4 consecutive weeks for active renal flare prior to treatment of Compound 1 or the pharmaceutically acceptable salt thereof.

65. The method of any one of claims 17-64, wherein the subject has not received treatment with prednisone ≥about 0.5 mg/kg/day or an equivalent thereof for 4 consecutive weeks for active renal flare prior to treatment of Compound 1 or the pharmaceutically acceptable salt thereof.

66. The method of any one of claims 17-65, wherein the subject does not have uncontrolled hypertension on 2 or more measurements within a 6-week period prior to treatment with Compound 1 or the pharmaceutically acceptable salt thereof.

67. The method of any one of claims 17-66, wherein the subject does not have a history of or has clinically active SLE-related cerebritis, seizures, pericarditis, stroke, or stroke syndrome requiring treatment.

68. The method of any one of claims 17-67, wherein the subject does not have an inability to take or tolerate treatment with a corticosteroid, MMF, or MPS.

69. The method of any one of claims 17-68, wherein the subject is restricted from receiving treatment with a calcineurin inhibitor.

70. The method of any one of claims 1-69, wherein the subject has IgAN.

71. The method of claim 70, wherein the subject has been diagnosed with primary IgAN confirmed by a kidney biopsy obtained prior to treatment.

72. The method of claim 71, wherein the kidney biopsy is obtained more than 2 years prior to treatment, and the subject has hematuria as defined by 1+ blood based on urine dipstick or ≤10 red blood cells (RBCs)/high-power field (hp) microscopy on urine sediment.

73. The method of any one of claims 70-72, wherein the subject has been receiving treatment with a stable and optimal dose of a RAS inhibitor prior to treatment with Compound 1 or the pharmaceutically acceptable salt thereof and continues to receive treatment with the RAS inhibitor during treatment with Compound 1 or the pharmaceutically acceptable salt thereof.

74. The method of claim 73, wherein the subject is restricted from receiving treatment with a second RAS inhibitor within a 50-week period following the start of treatment with Compound 1 or the pharmaceutically acceptable salt thereof.

75. The method of any one of claims 70-74, wherein the subject has been receiving treatment with a stable and optimal dose of a direct renin antagonist prior to treatment with Compound 1 or the pharmaceutically acceptable salt thereof and continues to receive treatment with the direct renin antagonist during treatment with Compound 1 or the pharmaceutically acceptable salt thereof.

76. The method of any one of claims 70-74, wherein the subject is restricted from receiving treatment with a second direct renin antagonist within a 50-week period following the start of treatment with Compound 1 or the pharmaceutically acceptable salt thereof.

77. The method of any one of claims 70-76, wherein the subject has controlled and stable blood pressure over a 3-month period prior to treatment with Compound 1 or the pharmaceutically acceptable salt thereof.

78. The method of any one of claims 70-77, wherein the subject experiences partial remission following a 50-week treatment period.

79. The method of any one of claims 70-78, wherein the subject experiences partial remission following a 26-week treatment period.

80. The method of any one of claims 70-79, wherein said treating comprises attenuating or flattening a slope of eGFR computed from baseline to week 26 of a treatment period as compared to a control.

81. The method of any one of claims 70-80, wherein the subject was not diagnosed with rapid progressive glomerulonephritis as measured by an eGFR loss ≥30% over a period of 3 months prior to treatment with Compound 1 or the pharmaceutically acceptable salt thereof.

82. The method of any one of claims 70-81, wherein the subject does not have a secondary etiology of IgAN.

83. The method of any one of claims 70-82, wherein the subject does not have clinically active Henoch-Schonlein purpura (IgA vasculitis) requiring treatment.

84. The method of any one of claims 70-83, wherein the subject has not received treatment with prednisone >about 20 mg/day or an equivalent thereof for >14 consecutive days or any other immunosuppression prior to treatment with Compound 1 or the pharmaceutically acceptable salt thereof.

85. The method of any one of claims 70-84, wherein the subject does not have blood pressure of ≥140/90 mmHg prior to treatment with Compound 1 or the pharmaceutically acceptable salt thereof confirmed on 2 measurements >30 minutes apart.

86. The method of any one of claims 70-85, wherein the subject does not have a body mass index ≥38 kg/m² prior to treatment with Compound 1 or the pharmaceutically acceptable salt thereof.

87. The method of any one of claims 70-86, wherein the subject is restricted from receiving treatment with hydroxychloroquine.

88. The method of any one of claims 70-87, wherein the subject is restricted from receiving treatment with an immunosuppressive agent.

89. The method of any one of claims 70-88, wherein the subject is restricted from receiving treatment with a systemic corticosteroid for >14 consecutive days.

90. The method of any one of claims 1-89, wherein said treating comprises decreasing plasma Bb fragment of complement factor B (Bb) concentration and serum alternative pathway (AP) activity.

91. The method of any one of claims 1-90, wherein said treating comprises reducing hematuria in the subject.

92. The method of claim 91, wherein said reducing hematuria in the subject comprises a decrease in red blood cells (RBC) in urine from baseline after a 50-week treatment period.

93. The method of claim 91 or 92, wherein said reducing hematuria in the subject comprises a decrease in red blood cells (RBC) in urine from baseline after a 26-week treatment period.

94. The method of any one of claims 91-93, wherein said reducing hematuria in the subject comprises achieving <10 RBCs/hpf.

95. The method of any one of claims 1-94, wherein said treating comprises improving a 36-Item Short Form Survey Instrument (SF-36) score in one or more of Physical Functioning, Physical, Bodily Pain, Vitality, General Health, Emotion, Mental Health, and Social Functioning from baseline.

96. The method of any one of claims 1-95, wherein said treating comprises improving a EuroQol 5-dimension 5-level questionnaire (EQ-5D-5L) score in one or more of mobility, usual activities, self-care, pain/discomfort, and anxiety/depression.

97. The method of any one of claims 1-96, wherein the subject has an eGFR<30 mL/min/1.73 m² as calculated using CKD-EPI.

98. The method of any one of claims 1-97, wherein the subject has less than 50% tubular atrophy, glomerular sclerosis, or crescent formation in glomeruli on the most recent kidney biopsy obtained prior to treatment.

99. The method of any one of claims 1-98, wherein the subject does not have a concomitant significant renal disease other than LN or IgAN on the most recent kidney biopsy obtained prior to treatment with Compound 1 or the pharmaceutically acceptable salt thereof.

100. The method of any one of claims 1-99, wherein the subject does not have a history of solid organ or bone marrow transplant.

101. The method of any one of claims 1-100, wherein the subject is restricted from receiving a solid organ or bone marrow transplant during a 50-week treatment period.

102. The method of any one of claims 1-101, wherein the subject has not had a splenectomy and does not have functional asplenia.

103. The method of any one of claims 1-102, wherein the subject does not have a history of seizure.

104. The method of any one of claims 1-103, wherein the subject does not have a known or suspected complement deficiency unless the complement deficiency is attributable to LN or IgAN.

105. The method of any one of claims 1-104, wherein the subject does not have a history of or have risk factors for Torsades de Pointes, a QT interval corrected using Fridericia's formula (QTcF)>450 msec when the subject is male or >470 msec when the subject is female, or is receiving medication known to significantly increase the corrected QT interval (QTc).

106. The method of any one of claims 1-104, wherein the subject does not have an alanine aminotransferase level of >2×ULN.

107. The method of any one of claims 1-106, wherein the subject does not have a direct bilirubin level of >2×ULN.

108. The method of any one of claims 1-107, wherein the subject has a hemoglobin A1C level of <7.0% prior to treatment with Compound 1 or the pharmaceutically acceptable salt thereof.

109. The method of any one of claims 1-108, wherein the subject does not have a known or suspected history of drug or alcohol abuse or dependence within 1 year prior to treatment.

110. The method of any one of claims 1-109, wherein the subject does not have a history of malignancy within 5 years prior to treatment, wherein the malignancy is not nonmelanoma skin cancer or carcinoma in situ of the cervix that has been treated with no evidence of recurrence.

111. The method of any one of claims 1-110, wherein the subject is not exhibiting signs of a hepatitis B viral infection with negative surface antibodies.

112. The method of any one of claims 1-111, wherein the subject is not exhibiting signs of a hepatitis C viral infection; or is exhibiting signs of a hepatitis C viral infection but has been successfully treated and has a documented sustained virologic response.

113. The method of any one of claims 1-112, wherein the subject is not exhibiting signs of a human immunodeficiency virus infection.

114. The method of any one of claims 1-113, wherein the subject does not have bone marrow insufficiency or thrombocytopenia.

115. The method of any one of claims 1-114, wherein the subject did not have an active systemic bacterial, viral or fungal infection within 14 days prior to treatment with Compound 1 or the pharmaceutically acceptable salt thereof.

116. The method of any one of claims 1-115, wherein the subject does not have a history of N meningitidis infection.

117. The method of any one of claims 1-116, wherein the subject is not receiving treatment with a biologic medication that may affect immune system functioning; or has stopped receiving treatment with the biologic medication, and 5 terminal half-lives of the biologic has elapsed prior to treatment with Compound 1 or the pharmaceutically acceptable salt thereof.

118. The method of any one of claims 1-117, wherein the subject has not received treatment with belimumab or rituximab <6 months prior to treatment with Compound 1 or the pharmaceutically acceptable salt thereof.

119. The method of any one of claims 1-118, wherein the subject has not received, or is not receiving treatment with a complement inhibitor other than Compound 1 or the pharmaceutically acceptable salt thereof.

120. The method of any one of claims 1-119, wherein the subject has not received treatment with a medication selected from a strong cytochrome P450, family 3, subfamily A (CYP3A) inhibitor; a moderate CYP3A inhibitor; a strong inducer of CYP3A; a moderate inducer of CYP3A; and a sensitive substrate of CYP3A, within the longer of two weeks or five half-lives of the medication prior to the treatment with Compound 1 or the pharmaceutically acceptable salt thereof.

121. The method of any one of claims 1-120, wherein the subject has not received treatment with a medication selected from meperidine, pethidine, a typical (1^{st} generation) antipsychotic, clozapine, olanzapine, lithium, a tricyclic antidepressant, bupropion, aminophylline, and theophylline.

122. The method of any one of claims 1-121, wherein the subject is not pregnant or breastfeeding.

123. The method of any one of claims 1-122, wherein the subject is restricted from consuming foods and beverages that inhibit CYP3A4 enzyme activity.

124. The method of any one of claims 1-123, wherein the subject is restricted from receiving treatment with eculizumab.

125. The method of any one of claims 1-124, wherein the subject has been receiving treatment with a sodium-glucose cotransporter-2 (SGLT-2) inhibitor prior to treatment with Compound 1 or the pharmaceutically acceptable salt thereof, and the dose of the SGLT-2 inhibitor does not change during treatment with Compound 1 or the pharmaceutically acceptable salt thereof.

126. The method of any one of claims 1-124, wherein the subject has not been receiving treatment with a SGLT-2 inhibitor prior to treatment with Compound 1 or the pharmaceutically acceptable salt thereof, and the subject is restricted from receiving treatment with a SGLT-2 inhibitor within a 50-week period following the start of treatment with Compound 1 or the pharmaceutically acceptable salt thereof.

127. The method of any one of claims 1-126, wherein the subject is restricted from using a medication selected from a strong CYP3A inhibitor, a moderate CYP3A inhibitor, a strong inducer of CYP3A, a moderate inducer of CYP3A, and a sensitive substrate of CYP3A.

128. The method of any one of claims 1-127, wherein the subject is restricted from using a medication selected from meperidine, pethidine, a typical (1^{st} generation) antipsychotic, clozapine, olanzapine, lithium, a tricyclic antidepressant, bupropion, aminophylline, and theophylline.

129. The method of any one of claims 1-128, wherein the subject is restricted from using a medication known to significantly prolong QTc, provided that the medication is not hydroxychloroquine for use by a subject with LN.

130. Use of Compound 1: 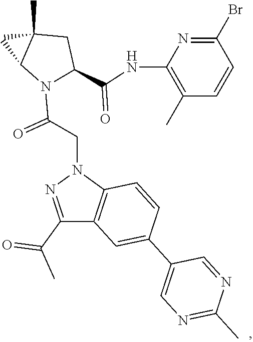 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof in the manufacture of a medicament for use in a method of treatment, wherein the method of treatment is the method of any one of claims 1-129.

131. A compound for use in a method of treatment, wherein the compound is Compound 1: 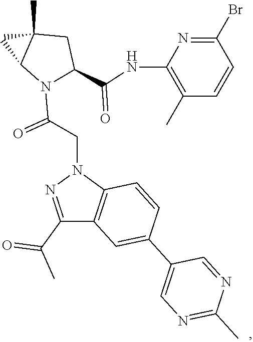 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof, wherein the method of treatment is the method of any one of claims 1-129.

132. A kit for treating LN or IgAN in a subject, comprising:
(a) a dose of Compound 1: 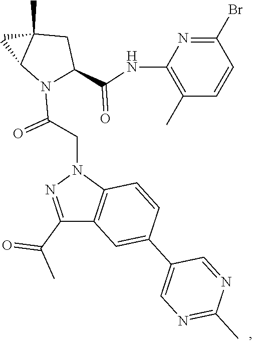 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof; and
(b) instructions for using Compound 1 or the pharmaceutically acceptable salt thereof according to the method of any one of claims 1-129.
